(19) United States
(12) Patent Application Publication
(10) Pub. No.: US 2022/0298107 A1
(43) Pub. Date: Sep. 22, 2022

(54) SUBSTITUTED UREA DIHYDROOROTATE DEHYDROGENASE INHIBITORS

(71) Applicant: Janssen Biotech, Inc., Horsham, PA (US)

(72) Inventors: Scott Kuduk, Harleysville, PA (US); Lindsey DeRatt, North Wales, PA (US)

(21) Appl. No.: 17/638,220

(22) PCT Filed: Aug. 27, 2020
(86) PCT No.: PCT/IB2020/058015

§ 371 Date: Feb. 25, 2022

Related U.S. Application Data
(60) Provisional application No. 62/893,204, filed on Aug. 29, 2019.

(51) Int. Cl. C07D 205/04; C07C 275/40; C07D 213/75; C07D 207/16; C07D 207/12; C07D 401/12; C07D 403/12

(52) U.S. Cl. CPC C07D 205/04; C07C 275/40; C07D 213/75; C07D 207/16; C07D 207/12; C07D 401/12; C07D 403/12

(57) ABSTRACT

Disclosed are compounds, compositions and methods for treating diseases, disorders, or medical conditions that are affected by the modulation of DHODH. Such compounds are represented by Formula I as follows:

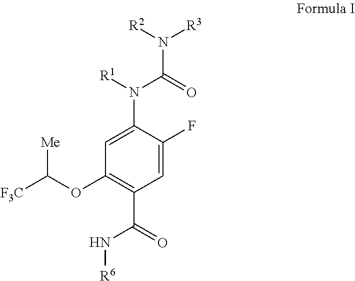

wherein R¹, R², R³ and R⁶ are defined herein.

### CROSS REFERENCE TO RELATED APPLICATIONS

This application claims priority to U.S. Provisional Patent Application No. 62/893,204, filed Aug. 29, 2019, the disclosure of which is hereby incorporated by reference in its entirety.

### FIELD OF THE INVENTION

The present invention relates to novel compounds that are dihydroorotate dehydrogenase (DHODH) inhibitors. These compounds may be useful for the treatment of a disease, disorder, or medical condition where there is an advantage in inhibiting DHODH. The invention also relates to pharmaceutical compositions comprising one or more of such compounds, to processes to prepare such compounds and compositions, and to the use of such compounds or pharmaceutical compositions for the method of treatment of cancer, and autoimmune and inflammatory diseases, syndromes, and disorders.

### BACKGROUND OF THE INVENTION

Acute myelogenous leukemia (AML) is a clonal disease of the blood and bone marrow resulting from mutations that occur in normal hematopoietic stem cells. AML is a heterogenous disease in that it presents with a range of cytogenetic, morphological and immunophenotypic features, and is characterized by an accumulation of clonal, abnormal myeloid progenitor cells, known as myeloblasts. These cells demonstrate disruption of normal myeloid differentiation and excessive proliferation, resulting in the decreased formation of hematopoietic cells. Disease remission can be achieved with standard induction chemotherapy, but refractory and relapsed disease remains a challenge due to persistence of leukemic stem cells. Therefore, AML represents an unmet medical need with >20,000 new cases per year in the US with 5-year overall survival below 30% (Stein E T et al., Health Qual Life Outcomes 16: 193, 2018).

Differentiation therapy is considered an attractive approach to AML treatment based on the knowledge that differentiation and loss of stem cell self-renewal are coupled in normal cells. Treatment of acute promyelocytic leukemia, which represents 10-15% of all AML, with all-trans retinoic acid is the paradigm for differentiation therapy. Retinoic acid targets the promyelocytic leukemia protein (PML)-retinoic acid receptor-α (RAR-α) fusion protein encoded by a t(15,17) chromosomal translocation. Targeting PML-RAR specifically lifts the transcriptionally mediated differentiation block induced by the fusion protein and early clinical trials with single agent ATRA demonstrated complete hematologic remission in all treated patients (McCulloch D et al. Onco Targets Ther 2017; 10: 1585-1601; Nowak D et al. Blood 113: 3655, 2009).

Although differentiation therapy is successful, it is only applicable to a small population of AML patients. Research efforts have aimed at identifying additional differentiation inducing agents, but with limited success. Recently dihydroorotate dehydrogenase (DHODH) emerged as a potentially more broadly applicable differentiation target in a phenotypic screen aimed at identifying small molecules that overcome blockade of the maturation of primary murine bone marrow cells expressing the homeobox protein HoxA9. This protein is a key transcription factor involved in balancing stem cell maintenance/differentiation and is normally expressed in hematopoietic progenitor cells and downregulated upon induction of differentiation and has been found to be widely overexpressed in AML (Sykes et al., Cell 167: 171, 2016).

DHODH is a flavin mononucleotide (FMN) flavoprotein located in the inner mitochondrial membrane that catalyzes the oxidation of dihydroorotate to orotate, the fourth step in the de novo pyrimidine biosynthesis pathway. Inhibition of DHODH leads to decreased pyrimidine synthesis important precursors for nucleotide synthesis, but also glycoprotein and phospholipid biosynthesis (Reis R A G et al., Archives Biochem Biophysics 632: 175, 2017; Vyas V K et al., Mini Rev Med Chem 11: 1039, 2011). DHODH is a validated target for the treatment of autoimmune diseases with the FDA approved small molecule DHODH inhibitors leflunomide and teriflunomide for rheumatoid arthritis and multiple sclerosis, respectively (Lolli M L et al., Recent patents on Anti-Cancer Drug Discovery 13: 86, 2018).

Since the first observation by Sykes et al. demonstrating that DHODH inhibition drives AML differentiation in vitro, as evidenced by upregulation of the differentiation markers CD11b and CD14, and results in dose dependent anti-leukemic effects, decreased leukemic stem cells and prolonged survival in vivo, additional evidence emerged demonstrating that small molecule DHODH inhibitors mediate antiproliferative activity against AML cells with concomitant cell cycle arrest, upregulation of CD11b and CD14, and induction of apoptosis (Wu D et al., Haematologica 103: 1472, 2018; Sainas S et al., J Med Chem 61: 6034, 2018; Cao L et al., Mol Cancer Ther, October 23rd Epub ahead of print). Moreover, preclinical solid tumor in vitro and in vivo models demonstrated effectiveness of DHODH inhibition and DHODH was identified as a synthetic lethality in PTEN and KRAS mutant solid tumors (Pharmacology and Therapeutics, Epub Oct. 19, 2018; Mathur D et al., Cancer Discovery 7: 1, 2017; Cell Chemical Biology 25: 1, 2018).

Thus, there remains a need for DHODH inhibitors that provide a therapeutic benefit to patients suffering from cancer and/or inflammatory and immunological diseases.

### SUMMARY OF THE INVENTION

Embodiments of the present invention relate to compounds, pharmaceutical compositions containing them, methods of making and purifying them, methods of using them as inhibitors of DHODH enzymatic activity and methods for using them in the treatment of a subject suffering from or diagnosed with a disease, disorder, or medical condition such as autoimmune or inflammatory disorders, or diseases such as cancer.

Embodiments of this invention include compounds of Formula I

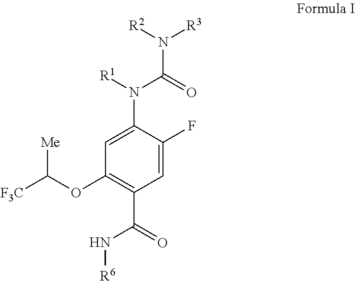

wherein:
R¹ is —H, or —C₍₁₋₄₎alkyl;
R² is —H, or —C₍₁₋₄₎alkyl, wherein said —C₍₁₋₄₎alkyl is optionally substituted with up to three fluorine atoms;
R³ is —C₍₁₋₄₎alkyl, or C₍₃₋₄₎cycloalkyl, wherein said —C₍₁₋₄₎alkyl is optionally substituted with —CN, —OCH₃, —OCF₃, —OH, or up to six fluorine atoms;
or R² and R³ may be taken together with their attached nitrogen, to form a ring selected from the group consisting of, azetidinyl, pyrrolidinyl, pyrrolidinonyl, morpholinyl, or piperidinyl, wherein said azetidinyl, pyrrolidinyl, pyrrolidinonyl, morpholinyl, or piperidinyl is optionally substituted with one or two fluorine atoms, or —OH, —OCH₃, —CF₃, —OCF₃, —OCHF₂, or —C₍₁₋₄₎alkyl, wherein said —C₍₁₋₄₎alkyl is optionally substituted with —OCH₃, —OCF₃, —OCHF₂, —OH, or up to six fluorine atoms;
R⁶ is selected from the group consisting of:

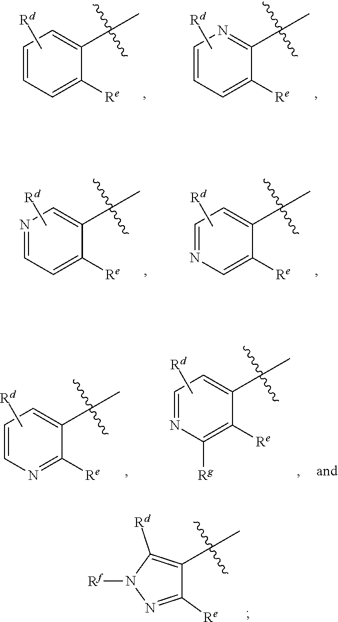

R^{d} is selected from the group consisting of: H; halo; C₁₋₆alkyl; C₁₋₆alkyl substituted with a member selected from the group consisting of: OH, OCH₃, SCH₃, and OCF₃; C₁₋₆haloalkyl; C₁₋₆haloalkyl substituted with a member selected from the group consisting of: OH, and OCH₃; and OC₁₋₆alkyl;
R^{e} is selected from the group consisting of: halo; C₁₋₆alkyl; C₁₋₆alkyl substituted with a member selected from the group consisting of: OH, OCH₃, SCH₃, and OCF₃; C₁₋₆haloalkyl; C₁₋₆haloalkyl substituted with a member selected from the group consisting of: OH, and OCH₃; and OC₁₋₆alkyl;

R^{f} is selected from the group consisting of: H; C₁₋₆alkyl; C₁₋₆alkyl substituted with a member selected from the group consisting of: OH, OCH₃, SCH₃, and OCF₃; C₁₋₆haloalkyl; and C₁₋₆haloalkyl substituted with a member selected from the group consisting of: OH, and OCH₃; and

R^{g} is selected from the group consisting of: H; C₁₋₆alkyl; C₁₋₆alkyl substituted with a member selected from the group consisting of: OH, OCH₃, SCH₃, and OCF₃; C₁₋₆haloalkyl; C₁₋₆haloalkyl substituted with a member selected from the group consisting of: OH, and OCH₃; and OC₁₋₆alkyl;
or a pharmaceutically acceptable salt, isotope, N-oxide, solvate, or stereoisomer thereof.

Embodiments of this invention include compounds of Formula II

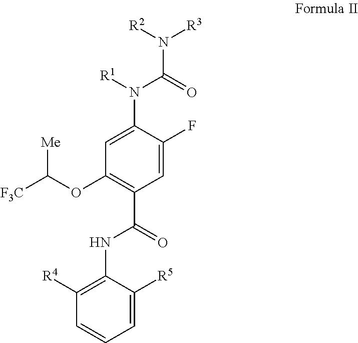

wherein:
R¹ is H, or —C₍₁₋₄₎alkyl;
R² is —H, or —C₍₁₋₄₎alkyl, wherein said —C₍₁₋₄₎alkyl is optionally substituted with up to three fluorine atoms;
R³ is —C₍₁₋₄₎alkyl, or C₍₃₋₄₎cycloalkyl, wherein said —C₍₁₋₄₎alkyl is optionally substituted with —CN, —OCH₃, —OCF₃, —OH, or up to six fluorine atoms;
or R² and R³ may be taken together with their attached nitrogen, to form a ring selected from the group consisting of, azetidinyl, pyrrolidinyl, pyrrolidinonyl, morpholinyl, or piperidinyl, wherein said azetidinyl, pyrrolidinyl, pyrrolidinonyl, morpholinyl, or piperidinyl is optionally substituted with one or two fluorine atoms, or —OH, —OCH₃, —CF₃, —OCF₃, —OCHF₂, or —C₍₁₋₄₎alkyl, wherein said —C₍₁₋₄₎alkyl is optionally substituted with —OCH₃, —OCF₃, —OCHF₂, —OH, or up to six fluorine atoms;
R⁴ and R⁵ are independently selected from —F, —Cl, —Br, and —I;
or a pharmaceutically acceptable salt, isotope, N-oxide, solvate, or stereoisomer thereof.

Embodiments of this invention include compounds of Formula III

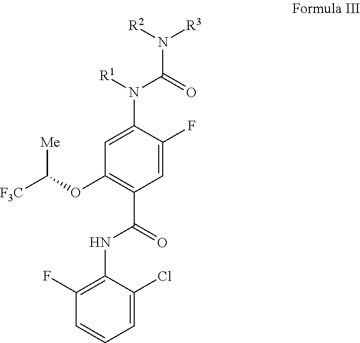

wherein:
R¹ is H, or —CH₃;
R² is —H, or —C₍₁₋₂₎alkyl, wherein said —C₍₁₋₂₎alkyl is optionally substituted with up to three fluorine atoms;
R³ is —C₍₁₋₄₎alkyl, or C₍₃₋₄₎cycloalkyl, wherein said —C₍₁₋₄₎alkyl is optionally substituted with —CN, —OCH₃, —OH, or up to six fluorine atoms;
or R² and R³ may be taken together with their attached nitrogen, to form a ring selected from the group consisting of, azetidinyl, pyrrolidinyl, pyrrolidinonyl, or piperidinyl, wherein said azetidinyl, pyrrolidinyl, pyrrolidinonyl, or piperidinyl is optionally substituted with one or two fluorine atoms, or —OH, —OCH₃, —CF₃, —OCF₃, —OCHF₂, or —C₍₁₋₄₎alkyl, wherein said —C₍₁₋₄₎alkyl is optionally substituted with —OCH₃, —OCF₃, —OCHF₂, —OH, or up to six fluorine atoms;
or a pharmaceutically acceptable salt, isotope, N-oxide, solvate, or stereoisomer thereof.

The present invention further provides methods for treating or ameliorating a disease, syndrome, condition, or disorder in a subject, including a mammal and/or human in which the disease, syndrome, condition, or disorder is affected by the inhibition of DHODH enzymatic activity, including but not limited to, cancer and/or inflammatory or immunological diseases, using a compound of Formula (I), (II) or (III), or a pharmaceutically acceptable salt, isotope, N-oxide, solvate, or stereoisomer thereof.

Additional embodiments, features, and advantages of the invention will be apparent from the following detailed description and through practice of the invention.

### DETAILED DESCRIPTION OF THE INVENTION

Unless defined otherwise, all technical and scientific terms used herein have the same meaning as is commonly understood by one of ordinary skill in art. As used in the specification and the appended claims, unless specified to the contrary, the following terms have the meaning indicated in order to facilitate the understanding of the present invention.

The singular forms “a”, “an” and “the” encompass plural references unless the context clearly indicates otherwise.

Unless qualified specifically in particular instances of use, the term “alkyl” refers to a straight- or branched-chain alkyl group having from 1 to 8 carbon atoms in the chain. Examples of alkyl groups include methyl (Me), ethyl (Et), n-propyl, isopropyl, butyl, isobutyl, sec-butyl, tert-butyl (tBu), pentyl, isopentyl, tert-pentyl, hexyl, isohexyl, and groups that in light of the ordinary skill in the art and the teachings provided herein would be considered equivalent to any one of the foregoing examples.

The term Cn-m alkyl refers to an aliphatic chain, whether straight or branched, with a total number N of carbon members in the chain that satisfies n≤N≤m, with m>n. For example and without limitation, the term “C₁₋₆alkyl” refers to straight- or branched-chain alkyl group having from 1 to 6 carbon atoms in the chain. “C₁₋₄alkyl” refers to straight- or branched-chain alkyl group having from 1 to 4 carbon atoms in the chain.

The term “halogen” or “halo” represents chlorine, fluorine, bromine, or iodine.

The term “haloalkyl” refers to a straight- or branched-chain alkyl group having from 1 to 6 carbon atoms in the chain optionally substituting hydrogens with halogens. The term “C₁₋₆ haloalkyl” as used here refers to a straight- or branched-chain alkyl group having from 1 to 6 carbon atoms in the chain, optionally substituting hydrogens with halogens. The term “C₁₋₄ haloalkyl” as used here refers to a straight- or branched-chain alkyl group having from 1 to 4 carbon atoms in the chain, optionally substituting hydrogens with halogens. Examples of “haloalkyl” groups include trifluoromethyl (CF₃), difluoromethyl (CF₂H), monofluoromethyl (CH₂F), pentafluoroethyl (CF₂CF₃), tetrafluoroethyl (CHFCF₃), monofluoroethyl (CH₂CH₂F), trifluoroethyl (CH₂CF₃), tetrafluorotrifluoromethylethyl (CF(CF₃)₂), and groups that in light of the ordinary skill in the art and the teachings provided herein would be considered equivalent to any one of the foregoing examples.

The term “cycloalkyl” refers to a saturated or partially saturated, monocyclic, fused polycyclic, or spiro polycyclic carbocycle having from 3 to 12 ring atoms per carbocycle. “C₃₋₆cycloalkyl” refers to a carbocycle having from 3 to 6 ring atoms per carbocycle. Illustrative examples of cycloalkyl groups include the following entities, in the form of properly bonded moieties.

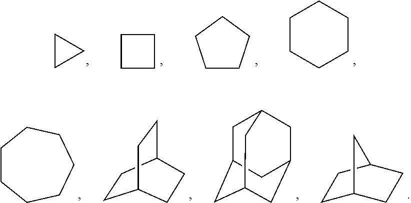

The term “azetidinyl” refers to the radical formed by removing a hydrogen atom from azetidine; for greater clarity, the term “azetidine” means

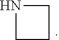

The term “pyrrolidinyl” refers to the radical formed by removing a hydrogen atom from pyrrolidine; for greater clarity, the term “pyrrolidine” means

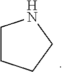

The term “pyrrolidinonyl” refers to the radical formed by removing a hydrogen atom from pyrrolidinone; for greater clarity, the term “pyrrolidinone” means

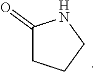

The term “piperidinyl” refers to the radical formed by removing a hydrogen atom from piperidine; for greater clarity, the term “piperidine” means

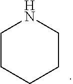

With reference to substituents, the term “independently” refers to the situation where when more than one substituent is possible, the substituents may be the same or different from each other.

The term “substituted” means that the specified group or moiety bears one or more substituents. The term “unsubstituted” means that the specified group bears no substituents. The term “optionally substituted” means that the specified group is unsubstituted or substituted by one or more substituents. Where the term “substituted” is used to describe a structural system, the substitution is meant to occur at any valency-allowed position on the system.

The term “variable point of attachment” means that a group is allowed to be attached at more than one alternative position in a structure. The attachment will always replace a hydrogen atom on one of the ring atoms. In other words, all permutations of bonding are represented by the single diagram, as shown in the illustrations below.

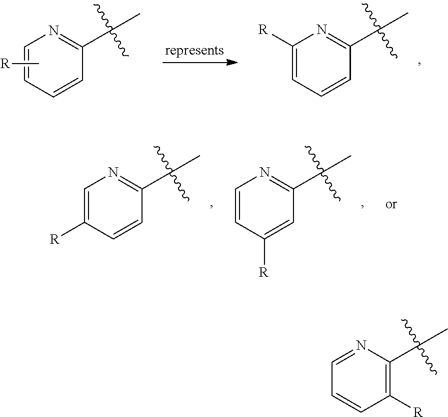

Those skilled in the art will recognize that that if more than one such substituent is present for a given ring, the bonding of each substituent is independent of all of the others. The groups listed or illustrated above are not exhaustive.

As used herein, the term “or” means “and/or” unless stated otherwise.

As used herein, the terms “including”, “containing” and “comprising” are used in their open, non-limiting sense.

As used herein, the term “composition” is intended to encompass a product comprising the specified ingredients in the specified amounts, as well as any product which results, directly or indirectly, from combination of the specified ingredients in the specified amounts.

As used herein, the term “treat”, “treating”, or “treatment” of any disease, condition, syndrome or disorder refers, in one embodiment, to ameliorating the disease, condition, syndrome or disorder (i.e. slowing or arresting or reducing the development of the disease or at least one of the clinical symptoms thereof). In another embodiment, “treat”, “treating”, or “treatment” refers to alleviating or ameliorating at least one physiological or biochemical parameter associated with or causative of the disease, condition, syndrome or disorder, including those which may not be discernible by the patient. In a further embodiment, “treat”, “treating”, or “treatment” refers to modulating the disease, condition, syndrome or disorder either physically (e.g. stabilization of a discernible symptom), physiologically, (e.g. stabilization of a physical parameter), or both. In yet another embodiment, “treat”, “treating”, or “treatment” refers to preventing or delaying the onset or development or progression of the disease, condition, syndrome or disorder.

The terms “subject” and “patient” are used interchangeably herein and may refer to an animal, preferably a mammal, most preferably a human.

As used herein, the terms active compound, pharmaceutical agent and active ingredient are used interchangeably to refer to a pharmaceutically active compound. Other ingredients in a drug composition, such as carriers, diluents or excipients, may be substantially or completely pharmaceutically inert. A pharmaceutical composition (also referred to herein as a composition or formulation) may comprise the active ingredient in combination with one or more carriers and/or one or more excipients and/or one or more diluents.

The term “therapeutically effective amount” (used interchangeably herein with “effective amount”) refers to an amount (e.g., of an active compound or pharmaceutical agent, such as a compound of the present invention), which elicits the biological or medicinal response in a tissue system, animal or human that is being sought by a researcher, veterinarian, medical doctor or other clinician, including reduction or inhibition of an enzyme or a protein activity, or ameliorating symptoms, alleviating conditions, slowing or delaying disease progression, or preventing a disease. Stated another way, the term therapeutically effective amount may refer to an amount that, when administered to a particular subject, achieves a therapeutic effect by inhibiting, alleviating or curing a disease, condition, syndrome or disorder in the subject or by prophylactically inhibiting, preventing or delaying the onset of a disease, condition, syndrome or disorder, or symptom(s) thereof. A therapeutically effective amount may be an amount which relieves to some extent one or more symptoms of a disease, condition, syndrome or disorder in a subject; and/or returns to normal either partially or completely one or more physiological or biochemical parameters associated with or causative of the disease, condition, syndrome or disorder; and/or reduces the likelihood of the onset of the disease, condition, syndrome or disorder, or symptom(s) thereof.

“Pharmaceutically acceptable” means that, which is useful in preparing a pharmaceutical composition that is generally safe, non-toxic and neither biologically nor otherwise undesirable and includes that which is acceptable for veterinary as well as human pharmaceutical use.

A “pharmaceutically acceptable salt” is intended to mean a salt of an acid or base of a compound represented by Formula (I) (as well as compounds of Formula (II) and (III)) that is non-toxic, biologically tolerable, or otherwise biologically suitable for administration to the subject. See, generally, S. M. Berge, et al., “Pharmaceutical Salts”, J. Pharm. Sci., 1977, 66:1-19, and Handbook of Pharmaceutical Salts, Properties, Selection, and Use, Stahl and Wermuth, Eds., Wiley-VCH and VHCA, Zurich, 2002. Preferred pharmaceutically acceptable salts are those that are pharmacologically effective and suitable for contact with the tissues of patients without undue toxicity, irritation, or allergic response.

Non-limiting examples of pharmaceutically acceptable salts include sulfates, pyrosulfates, bisulfates, sulfites, bisulfites, phosphates, monohydrogen-phosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, xylenesulfonates, phenylacetates, phenylpropionates, phenylbutyrates, citrates, lactates, γ-hydroxybutyrates, glycolates, tartrates, methane-sulfonates, propanesulfonates, naphthalene-1-sulfonates, naphthalene-2-sulfonates, and mandelates.

A compound of Formula (I), (II) or (III) may possess a sufficiently acidic group, a sufficiently basic group, or both types of functional groups, and accordingly react with a number of inorganic or organic bases, and inorganic and organic acids, to form a pharmaceutically acceptable salt.

Compounds of Formula (I), (II) or (III) may contain at least one nitrogen of basic character, so desired pharmaceutically acceptable salts may be prepared by any suitable method available in the art, for example, treatment of the free base with an inorganic acid, such as hydrochloric acid, hydrobromic acid, sulfuric acid, sulfamic acid, nitric acid, boric acid, phosphoric acid, and the like, or with an organic acid, such as acetic acid, phenylacetic acid, propionic acid, stearic acid, lactic acid, ascorbic acid, maleic acid, hydroxymaleic acid, isethionic acid, succinic acid, valeric acid, fumaric acid, malonic acid, pyruvic acid, oxalic acid, glycolic acid, salicylic acid, oleic acid, palmitic acid, lauric acid, a pyranosidyl acid, such as glucuronic acid or galacturonic acid, an alpha-hydroxy acid, such as mandelic acid, citric acid, or tartaric acid, an amino acid, such as aspartic acid or glutamic acid, an aromatic acid, such as benzoic acid, 2-acetoxybenzoic acid, naphthoic acid, or cinnamic acid, a sulfonic acid, such as laurylsulfonic acid, p-toluenesulfonic acid, methanesulfonic acid, ethanesulfonic acid, any compatible mixture of acids such as those given as examples herein, and any other acid and mixture thereof that are regarded as equivalents.

Compounds of Formula (I), (II) or (III) may contain a carboxylic acid moiety, a desired pharmaceutically acceptable salt may be prepared by any suitable method, for example, treatment of the free acid with an inorganic or organic base, such as an amine (primary, secondary or tertiary), an alkali metal hydroxide, alkaline earth metal hydroxide, any compatible mixture of bases such as those given as examples herein, and any other base and mixture thereof that are regarded as equivalents or acceptable substitutes in light of the ordinary level of skill in this technology. Illustrative examples of suitable salts include organic salts derived from amino acids, such as glycine and arginine, ammonia, carbonates, bicarbonates, primary, secondary, and tertiary amines, and cyclic amines, such as benzylamines, pyrrolidines, piperidine, morpholine, piperazine, N-methyl-glucamine and tromethamine and inorganic salts derived from sodium, calcium, potassium, magnesium, manganese, iron, copper, zinc, aluminum, and lithium.

Each compound used herein may be discussed interchangeably with respect to its chemical formula, chemical name, abbreviation, etc.

Any formula given herein is intended to represent compounds having structures depicted by the structural formula as well as certain variations or forms. In particular, compounds of any formula given herein may have asymmetric centers and therefore exist in different enantiomeric forms. All optical isomers and stereoisomers of the compounds of the general formula, and mixtures thereof, are considered within the scope of such formula. The compounds of this invention may possess one or more asymmetric centers; such compounds can therefore be produced as individual (R)- or (S)-stereoisomers or as mixtures thereof. Thus, any formula given herein is intended to represent a racemate, one or more of its enantiomeric forms, one or more of its diastereomeric forms, and mixtures thereof. Additionally, any formula given herein is intended to refer also to any one of hydrates, solvates, polymorphs and of such compounds, and mixtures thereof, even if such forms are not listed explicitly.

The term “R” at a stereocenter designates that the stereocenter is purely of the R-configuration as defined in the art; likewise, the term “S” means that the stereocenter is purely of the S-configuration. As used herein, the term “RS” refers to a stereocenter that exists as a mixture of the R- and S-configurations.

Compounds containing one stereocenter drawn without a stereo bond designation are a mixture of 2 enantiomers. Compounds containing 2 stereocenters both drawn without stereo bond designations are a mixture of 4 diastereomers. Compounds with 2 stereocenters both labeled “RS” and drawn with stereo bond designations are a 2-component mixture with relative stereochemistry as drawn. Unlabeled stereocenters drawn without stereo bond designations are a mixture of the R- and S-configurations. For unlabeled stereocenters drawn with stereo bond designations, the absolute stereochemistry is as depicted.

Unless indicated otherwise, the description or naming of a particular compound in the specification and claims is intended to include both individual enantiomers and mixtures, racemic or otherwise, thereof. The methods for the determination of stereochemistry and the separation of stereoisomers are well-known in the art.

Reference to a compound herein stands for a reference to any one of: (a) the recited form of such compound, and (b) any of the forms of such compound in the medium in which the compound is being considered when named. For example, reference herein to a compound such as R—COOH, encompasses reference to any one of, for example, R—COOH(s), R—COOH(sol), and R—COO-(sol). In this example, R—COOH(s) refers to the solid compound, as it could be for example in a tablet or some other solid pharmaceutical composition or preparation; R—COOH(sol) refers to the undissociated form of the compound in a solvent; and R—COO-(sol) refers to the dissociated form of the compound in a solvent, such as the dissociated form of the compound in an aqueous environment, whether such dissociated form derives from R—COOH, from a salt thereof, or from any other entity that yields R—COO— upon dissociation in the medium being considered. In another example, an expression such as “exposing an entity to compound of formula R—COOH” refers to the exposure of such entity to the form, or forms, of the compound R—COOH that exists, or exist, in the medium in which such exposure takes place. In still another example, an expression such as “reacting an entity with a compound of formula R—COOH” refers to the reacting of (a) such entity in the chemically relevant form, or forms, of such entity that exists, or exist, in the medium in which such reacting takes place, with (b) the chemically relevant form, or forms, of the compound R—COOH that exists, or exist, in the medium in which such reacting takes place. In this regard, if such entity is for example in an aqueous environment, it is understood that the compound R—COOH is in such same medium, and therefore the entity is being exposed to species such as R—COOH(aq) and/or R—COO-(aq), where the subscript “(aq)” stands for “aqueous” according to its conventional meaning in chemistry and biochemistry. A carboxylic acid functional group has been chosen in these nomenclature examples; this choice is not intended, however, as a limitation but it is merely an illustration. It is understood that analogous examples can be provided in terms of other functional groups, including but not limited to hydroxyl, basic nitrogen members, such as those in amines, and any other group that interacts or transforms according to known manners in the medium that contains the compound. Such interactions and transformations include, but are not limited to, dissociation, association, tautomerism, solvolysis, including hydrolysis, solvation, including hydration, protonation, and deprotonation. No further examples in this regard are provided herein because these interactions and transformations in a given medium are known by any one of ordinary skill in the art.

Any formula given herein is also intended to represent unlabeled forms as well as isotopically labeled forms of the compounds. Isotopically labeled compounds have structures depicted by the formulas given herein except that one or more atoms are replaced by an atom having a selected atomic mass or mass number in an enriched form. Examples of isotopes that can be incorporated into compounds of the invention in a form that exceeds natural abundances include isotopes of hydrogen, carbon, nitrogen, oxygen, phosphorous, fluorine, chlorine, and iodine, such as ²H (or chemical symbol D), ³H (or chemical symbol T), ¹¹C, ¹³C, ¹⁴C, ¹⁵N, ¹⁸O, ¹⁷O ³¹P, ³²P ³⁵S, ¹⁸F, ³⁶Cl, and ¹²⁵I, respectively. Such isotopically labelled compounds are useful in metabolic studies (preferably with ¹⁴C), reaction kinetic studies (with, for example ²H or ³H), detection or imaging techniques [such as positron emission tomography (PET) or single-photon emission computed tomography (SPECT)] including drug or substrate tissue distribution assays, or in radioactive treatment of patients. In particular, an ¹⁸F or ¹¹C labeled compound may be particularly preferred for PET or SPECT studies. Further, substitution with heavier isotopes such as deuterium (i.e., ²H, or D) may afford certain therapeutic advantages resulting from greater metabolic stability, for example increased in vivo half-life or reduced dosage requirements. Isotopically labeled compounds of this invention can generally be prepared by carrying out the procedures disclosed in the schemes or in the examples and preparations described below by substituting a readily available isotopically labeled reagent for a non-isotopically labeled reagent.

When the same plurality of substituents is assigned to various groups, the specific individual substituent assignment to each of such groups is meant to be independently made with respect to the specific individual substituent assignments to the remaining groups. By way of illustration, but not as a limitation, if each of groups Q and R can be H or F, the choice of H or F for Q is made independently of the choice of H or F for R, so the choice of assignment for Q does not determine or condition the choice of assignment for R, or vice-versa, unless it is expressly indicated otherwise. Illustrative claim recitation in this regard would read as “each of Q and R is independently H or F”, or “each of Q and R is independently selected from the group consisting of H and F”.

In another example, a zwitterionic compound would be encompassed herein by referring to a compound that is known to form a zwitterion, even if it is not explicitly named in its zwitterionic form. Terms such as zwitterion, zwitterions, and their synonyms zwitterionic compound(s) are standard IUPAC-endorsed names that are well known and part of standard sets of defined scientific names. In this regard, the name zwitterion is assigned the name identification CHEBI:27369 by the Chemical Entities of Biological Interest (ChEBI) dictionary of molecular entities. As generally well known, a zwitterion or zwitterionic compound is a neutral compound that has formal unit charges of opposite sign. Sometimes these compounds are referred to by the term “inner salts”. Other sources refer to these compounds as “dipolar ions”, although the latter term is regarded by still other sources as a misnomer. As a specific example, aminoethanoic acid (the amino acid glycine) has the formula H₂NCH₂COOH, and it exists in some media (in this case in neutral media) in the form of the zwitterion ⁺H₃NCH₂COO⁻. Zwitterions, zwitterionic compounds, inner salts and dipolar ions in the known and well-established meanings of these terms are within the scope of this invention, as would in any case be so appreciated by those of ordinary skill in the art. Because there is no need to name each and every embodiment that would be recognized by those of ordinary skill in the art, no structures of the zwitterionic compounds that are associated with the compounds of this invention are given explicitly herein. They are, however, part of the embodiments of this invention. No further examples in this regard are provided herein because the interactions and transformations in a given medium that lead to the various forms of a given compound are known by any one of ordinary skill in the art.

When referring to any formula given herein, the selection of a particular moiety from a list of possible species for a specified variable is not intended to define the same choice of the species for the variable appearing elsewhere. In other words, where a variable appears more than once, the choice of the species from a specified list is independent of the choice of the species for the same variable elsewhere in the formula, unless stated otherwise.

By way of a first example on substituent terminology, if substituent S¹ₑₓₐₘₚₗₑ is one of S₁ and S₂, and substituent S²ₑₓₐₘₚₗₑ is one of S₃ and S₄, then these assignments refer to embodiments of this invention given according to the choices S¹ₑₓₐₘₚₗₑ is S₁ and S²ₑₓₐₘₚₗₑ is S₃; S¹ₑₓₐₘₚₗₑ is S₁ and S²ₑₓₐₘₚₗₑ is S₄; S¹ₑₓₐₘₚₗₑ is S₂ and S²ₑₓₐₘₚₗₑ is S₃; S¹ₑₓₐₘₚₗₑ is S₂ and S²ₑₓₐₘₚₗₑ is S₄; and equivalents of each one of such choices. The shorter terminology “S¹ₑₓₐₘₚₗₑ is one of S₁ and S₂, and S²ₑₓₐₘₚₗₑ is one of S₃ and S₄” is accordingly used herein for the sake of brevity, but not by way of limitation. The foregoing first example on substituent terminology, which is stated in generic terms, is meant to illustrate the various substituent assignments described herein.

Furthermore, when more than one assignment is given for any member or substituent, embodiments of this invention comprise the various groupings that can be made from the listed assignments, taken independently, and equivalents thereof. By way of a second example on substituent terminology, if it is herein described that substituent Sₑₓₐₘₚₗₑ is one of S₁, S₂, and S₃, this listing refers to embodiments of this invention for which Sₑₓₐₘₚₗₑ is S₁; Sₑₓₐₘₚₗₑ is S₂; Sₑₓₐₘₚₗₑ is S₃; Sₑₓₐₘₚₗₑ is one of S₁ and S₂; Sₑₓₐₘₚₗₑ is one of S₁ and S₃; Sₑₓₐₘₚₗₑ is one of S₂ and S₃; Sₑₓₐₘₚₗₑ is one of S₁, S₂ and S₃; and Sₑₓₐₘₚₗₑ is any equivalent of each one of these choices. The shorter terminology “Sₑₓₐₘₚₗₑ is one of S₁, S₂, and S₃” is accordingly used herein for the sake of brevity, but not by way of limitation. The foregoing second example on substituent terminology, which is stated in generic terms, is meant to illustrate the various substituent assignments described herein.

Embodiments of this invention include compounds of Formula I

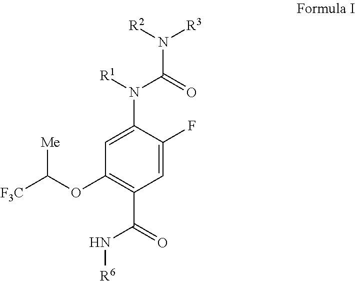

wherein:
R¹ is —H, or —C₍₁₋₄₎alkyl;
R² is —H, or —C₍₁₋₄₎alkyl, wherein said —C₍₁₋₄₎alkyl is optionally substituted with up to three fluorine atoms;
R³ is —C₍₁₋₄₎alkyl, or C₍₃₋₄₎cycloalkyl, wherein said —C₍₁₋₄₎alkyl is optionally substituted with —CN, —OCH₃, —OCF₃, —OH, or up to six fluorine atoms;
or R² and R³ may be taken together with their attached nitrogen, to form a ring selected from the group consisting of, azetidinyl, pyrrolidinyl, pyrrolidinonyl, morpholinyl, or piperidinyl, wherein said azetidinyl, pyrrolidinyl, pyrrolidinonyl, morpholinyl, or piperidinyl is optionally substituted with one or two fluorine atoms, or —OH, —OCH₃, —CF₃, —OCF₃, —OCHF₂, or —C₍₁₋₄₎alkyl, wherein said —C₍₁₋₄₎alkyl is optionally substituted with —OCH₃, —OCF₃, —OCHF₂, —OH, or up to six fluorine atoms;
R⁶ is selected from the group consisting of:

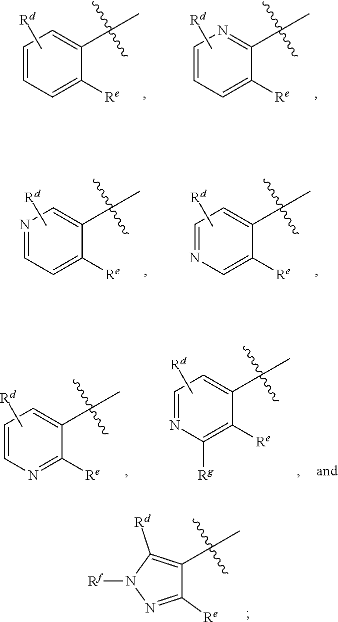

R^{d} is selected from the group consisting of: H; halo; C₁₋₆alkyl; C₁₋₆alkyl substituted with a member selected from the group consisting of: OH, OCH₃, SCH₃, and OCF₃; C₁₋₆haloalkyl; C₁₋₆haloalkyl substituted with a member selected from the group consisting of: OH, and OCH₃; and OC₁₋₆alkyl;
R^{e} is selected from the group consisting of: halo; C₁₋₆alkyl; C₁₋₆alkyl substituted with a member selected from the group consisting of: OH, OCH₃, SCH₃, and OCF₃; C₁₋₆haloalkyl; C₁₋₆haloalkyl substituted with a member selected from the group consisting of: OH, and OCH₃; and OC₁₋₆alkyl;
R^{f} is selected from the group consisting of: H; C₁₋₆alkyl; C₁₋₆alkyl substituted with a member selected from the group consisting of: OH, OCH₃, SCH₃, and OCF₃; C₁₋₆haloalkyl; and C₁₋₆haloalkyl substituted with a member selected from the group consisting of: OH, and OCH₃; and
R^{g} is selected from the group consisting of: H; C₁₋₆alkyl; C₁₋₆alkyl substituted with a member selected from the group consisting of: OH, OCH₃, SCH₃, and OCF₃; C₁₋₆haloalkyl; C₁₋₆haloalkyl substituted with a member selected from the group consisting of: OH, and OCH₃; and OC₁₋₆alkyl;

or a pharmaceutically acceptable salt, isotope, N-oxide, solvate, or stereoisomer thereof.

An additional embodiment of the invention is a compound of Formula (I) wherein R^{d} is selected from the group consisting of: H; halo; C₁₋₆alkyl; and OC₁₋₆alkyl; R^{e} is selected from the group consisting of: halo; C₁₋₆alkyl; and OC₁₋₆alkyl; R^{f} is selected from the group consisting of: H; and C₁₋₆alkyl; and R⁹ is selected from the group consisting of: H; C₁₋₆alkyl; and OC₁₋₆alkyl.

An additional embodiment of the invention is a compound of Formula (I) wherein R^{d} is selected from the group consisting of: H; halo; CH₃; and OCH₃; R^{e} is selected from the group consisting of: halo; CH₃; and OCH₃; R^{f} is selected from the group consisting of: H and CH₃; and R⁹ is selected from the group consisting of: H; CH₃; and OCH₃.

An embodiment of the invention is a compound of Formula (I) wherein R^{d} is selected from the group consisting of: H; Cl; CH₃; and OCH₃; R^{e} is selected from the group consisting of: Cl; CH₃; and OCH₃; R^{f} is selected from the group consisting of: H and CH₃; and R⁹ is selected from the group consisting of: H; CH₃; and OCH₃.

An additional embodiment of the invention is a compound of Formula (I) wherein R⁶ is

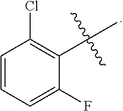 [SMILES: *c1c(F)cccc1Cl]

Embodiments of this invention also include compounds of Formula II

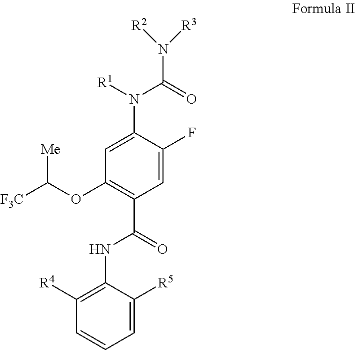

wherein:
R¹ is H, or —C₍₁₋₄₎alkyl;
R² is —H, or —C₍₁₋₄₎alkyl, wherein said —C₍₁₋₄₎alkyl is optionally substituted with up to three fluorine atoms;
R³ is —C₍₁₋₄₎alkyl, or C₍₃₋₄₎cycloalkyl, wherein said —C₍₁₋₄₎alkyl is optionally substituted with —CN, —OCH₃, —OCF₃, —OH, or up to six fluorine atoms;
or R² and R³ may be taken together with their attached nitrogen, to form a ring selected from the group consisting of, azetidinyl, pyrrolidinyl, pyrrolidinonyl, morpholinyl, or piperidinyl, wherein said azetidinyl, pyrrolidinyl, pyrrolidinonyl, morpholinyl, or piperidinyl is optionally substituted with one or two fluorine atoms, or —OH, —OCH₃, —CF₃, —OCF₃, —OCHF₂, or —C₍₁₋₄₎alkyl, wherein said —C₍₁₋₄₎alkyl is optionally substituted with —OCH₃, —OCF₃, —OCHF₂, —OH, or up to six fluorine atoms;
R⁴ and R⁵ are independently selected from —F, —Cl, —Br, and —I;
or a pharmaceutically acceptable salt, isotope, N-oxide, solvate, or stereoisomer thereof.

Embodiments of this invention also include compounds of Formula III,

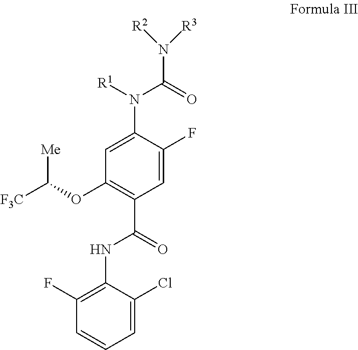

wherein:
R¹ is H, or —CH₃;
R² is —H, or —C₍₁₋₂₎alkyl, wherein said —C₍₁₋₂₎alkyl is optionally substituted with up to three fluorine atoms;
R³ is —C₍₁₋₄₎alkyl, or C₍₃₋₄₎cycloalkyl, wherein said —C₍₁₋₄₎alkyl is optionally substituted with —CN, —OCH₃, —OH, or up to six fluorine atoms;
or R² and R³ may be taken together with their attached nitrogen, to form a ring selected from the group consisting of, azetidinyl, pyrrolidinyl, pyrrolidinonyl, or piperidinyl, wherein said azetidinyl, pyrrolidinyl, pyrrolidinonyl, or piperidinyl is optionally substituted with one or two fluorine atoms, or —OH, —OCH₃, —CF₃, —OCF₃, —OCHF₂, or —C₍₁₋₄₎alkyl, wherein said —C₍₁₋₄₎alkyl is optionally substituted with —OCH₃, —OCF₃, —OCHF₂, —OH, or up to six fluorine atoms;
or a pharmaceutically acceptable salt, isotope, N-oxide, solvate, or stereoisomer thereof.

Another embodiment of the invention is a compound of Formula III, wherein

R¹ is H, or —CH₃;
R² is —H, or —C₍₁₋₂₎alkyl;
R³ is —C₍₁₋₄₎alkyl, or C₍₃₋₄₎cycloalkyl, wherein said —C₍₁₋₄₎alkyl is optionally substituted with —CN, —OCH₃, —OH, or up to three fluorine atoms;
or R² and R³ may be taken together with their attached nitrogen, to form a ring selected from the group consisting of, azetidinyl, pyrrolidinyl, pyrrolidinonyl, or piperidinyl, wherein said azetidinyl, pyrrolidinyl, pyrrolidinonyl, or piperidinyl is optionally substituted with one or two fluorine atoms, or —OH, —OCH₃, —OCF₃, —OCHF₂, or —C₍₁₋₄₎alkyl, wherein said —C₍₁₋₄₎alkyl is optionally substituted with —OCH₃, —OCF₃, —OCHF₂, —OH, or up to two fluorine atoms;
or a pharmaceutically acceptable salt, isotope, N-oxide, solvate, or stereoisomer thereof.

Another embodiment of the invention is a compound of Formula III, wherein

R¹ is H, or —CH₃;
R² is —H, or —C₍₁₋₂₎alkyl;
R³ is —C₍₁₋₄₎alkyl, or C₍₃₋₄₎cycloalkyl, wherein said —C₍₁₋₄₎alkyl is optionally substituted with —CN, —OCH₃, —OH, or up to two fluorine atoms;
or R² and R³ may be taken together with their attached nitrogen, to form a ring selected from the group consisting of, azetidinyl, pyrrolidinyl, pyrrolidinonyl, or piperidinyl, wherein said azetidinyl, pyrrolidinyl, pyrrolidinonyl, or piperidinyl is optionally substituted with one or two fluorine atoms, or —OH, —OCH₃, or —C₍₁₋₄₎alkyl, wherein said —C₍₁₋₄₎alkyl is optionally substituted with —OCH₃, —OH, or up to two fluorine atoms;
or a pharmaceutically acceptable salt, isotope, N-oxide, solvate, or stereoisomer thereof.

Another embodiment of the invention is a compound of Formula III, wherein

R¹ is H, or —CH₃;
R² is —H, or —C₍₁₋₂₎alkyl;
R³ is —C₍₁₋₃₎alkyl, or cyclopropyl, wherein said —C₍₁₋₃₎alkyl is optionally substituted with —CN, —OCH₃, —OH, or up to two fluorine atoms;
or R² and R³ may be taken together with their attached nitrogen, to form a ring selected from the group consisting of, azetidinyl, pyrrolidinyl, pyrrolidinonyl, or piperidinyl, wherein said azetidinyl, pyrrolidinyl, pyrrolidinonyl, or piperidinyl is optionally substituted with one or two fluorine atoms, or —OH, —OCH₃, or —C₍₁₋₃₎alkyl, wherein said —C₍₁₋₃₎alkyl is optionally substituted with —OCH₃, —OH, or up to two fluorine atoms;
or a pharmaceutically acceptable salt, isotope, N-oxide, solvate, or stereoisomer thereof.

Another embodiment of the invention is a compound of Formula III, selected from the group consisting of:

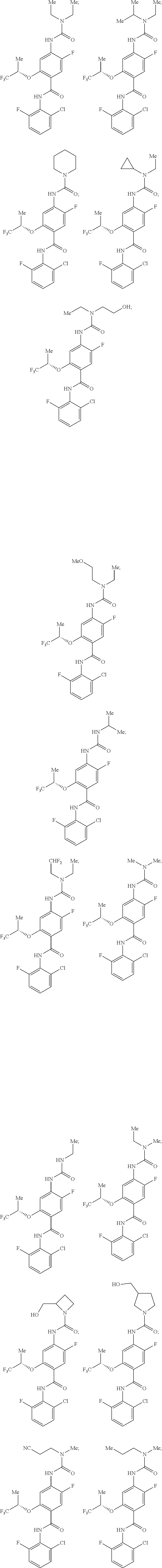

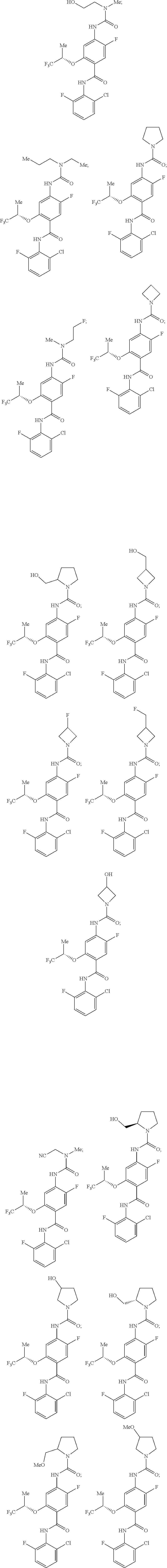

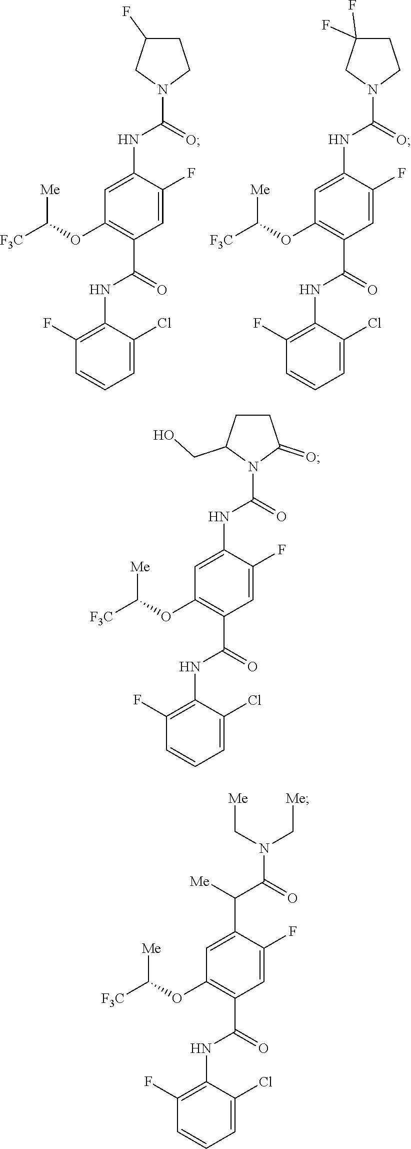

or a pharmaceutically acceptable salt, isotope, N-oxide, solvate, or stereoisomer thereof.

Another embodiment of the invention is a compound of Formula I selected from the group consisting of:

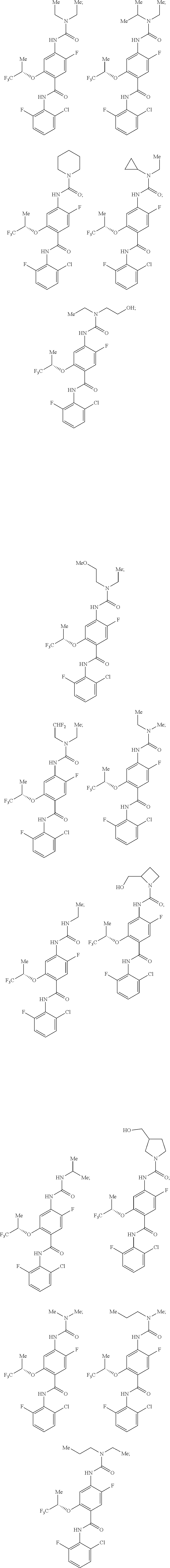

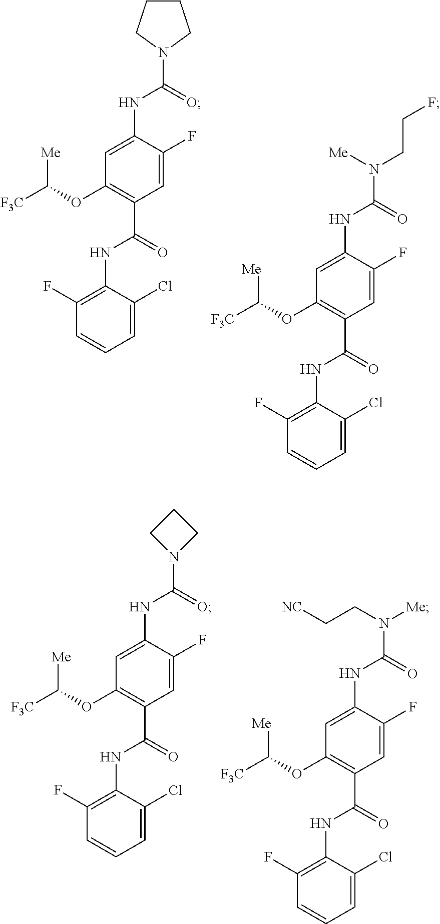 [SMILES: C[C@H](Oc1cc(NC(=O)N2CCCC2)c(F)cc1C(=O)Nc1c(F)cccc1Cl)C(F)(F)F ; C[C@H](Oc1cc(NC(=O)N(C)CCF)c(F)cc1C(=O)Nc1c(F)cccc1Cl)C(F)(F)F ; C[C@H](Oc1cc(NC(=O)N2CCC2)c(F)cc1C(=O)Nc1c(F)cccc1Cl)C(F)(F)F ; C[C@H](Oc1cc(NC(=O)N(C)CCC#N)c(F)cc1C(=O)Nc1c(F)cccc1Cl)C(F)(F)F]

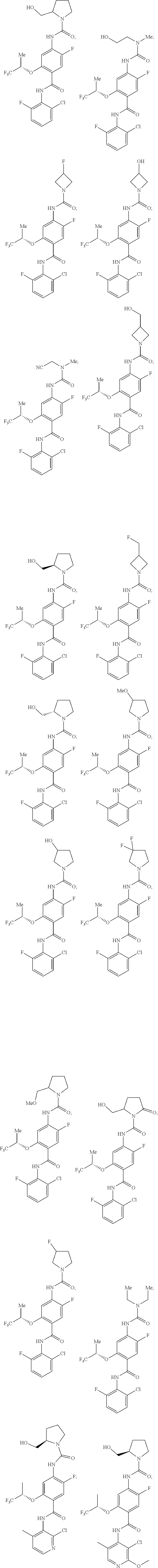

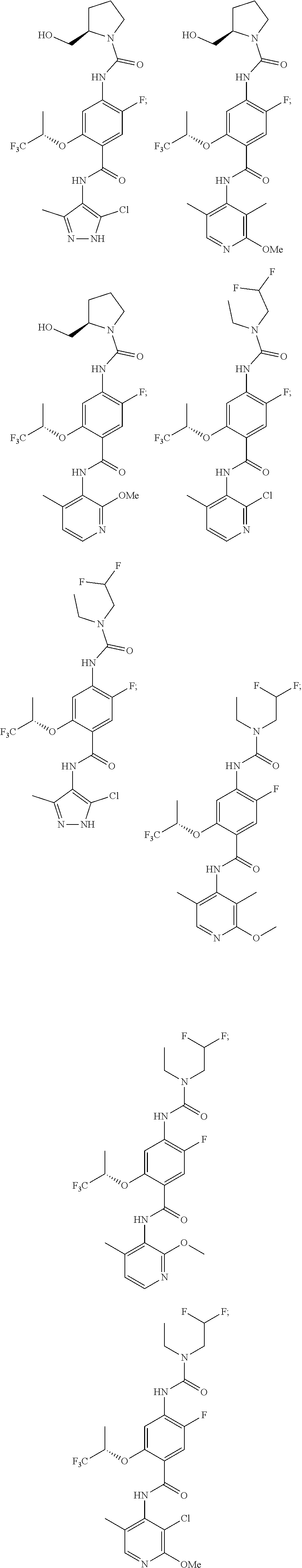

or a pharmaceutically acceptable salt, isotope, N-oxide, solvate, or stereoisomer thereof.

A further embodiment of the current invention is a compound as shown below in Table 1, or pharmaceutically acceptable salts, isotopes, tautomers, N-oxides, solvates, or stereoisomers thereof.

TABLE 1

| Example # | Compound Name |
|---|---|
| 1 | (S)-N-(2-chloro-6-fluorophenyl)-4-(3,3-diethylureido)-5-fluoro-2-((1,1,1- |
| | trifluoropropan-2-yl)oxy)benzamide; |
| 2 | (S)-N-(4-((2-chloro-6-fluorophenyl)carbamoyl)-2-fluoro-5-((1,1,1- |
| | trifluoropropan-2-yl)oxy)phenyl)piperidine-1-carboxamide; |
| 3 | (S)-N-(2-chloro-6-fluorophenyl)-4-(3-ethyl-3-(2-hydroxyethyl)ureido)-5-fluoro- |
| | 2-((1,1,1-trifluoropropan-2-yl)oxy)benzamide; |
| 4 | (S)-N-(2-chloro-6-fluorophenyl)-4-(3-ethyl-3-isopropylureido)-5-fluoro-2- |
| | ((1,1,1-trifluoropropan-2-yl)oxy)benzamide; |
| 5 | (S)-N-(2-chloro-6-fluorophenyl)-4-(3-cyclopropyl-3-ethylureido)-5-fluoro-2- |
| | ((1,1,1-trifluoropropan-2-yl)oxy)benzamide; |
| 6 | (S)-N-(2-chloro-6-fluorophenyl)-4-(3-ethyl-3-(2-methoxyethyl)ureido)-5-fluoro- |
| | 2-((1,1,1-trifluoropropan-2-yl)oxy)benzamide; |
| 7 | (S)-N-(2-chloro-6-fluorophenyl)-4-(3-(2,2-difluoroethyl)-3-ethylureido)-5- |
| | fluoro-2-((1,1,1-trifluoropropan-2-yl)oxy)benzamide; |
| 8 | (S)-N-(2-chloro-6-fluorophenyl)-4-(3-ethylureido)-5-fluoro-2-((1,1,1- |
| | trifluoropropan-2-yl)oxy)benzamide; |
| 9 | (S)-N-(2-chloro-6-fluorophenyl)-5-fluoro-4-(3-isopropylureido)-2-((1,1,1- |
| | trifluoropropan-2-yl)oxy)benzamide; |
| 10 | (S)-N-(2-chloro-6-fluorophenyl)-4-(3,3-dimethylureido)-5-fluoro-2-((1,1,1- |
| | trifluoropropan-2-yl)oxy)benzamide; |
| 11 | (S)-N-(2-chloro-6-fluorophenyl)-4-(3-ethyl-3-methylureido)-5-fluoro-2-((1,1,1- |
| | trifluoropropan-2-yl)oxy)benzamide; |
| 12 | N-(4-((2-chloro-6-fluorophenyl)carbamoyl)-2-fluoro-5-(((S)-1,1,1- |
| | trifluoropropan-2-yl)oxy)phenyl)-2-(hydroxymethyl)azetidine-1-carboxamide; |
| 13 | N-(4-((2-chloro-6-fluorophenyl)carbamoyl)-2-fluoro-5-(((S)-1,1,1- |
| | trifluoropropan-2-yl)oxy)phenyl)-3-(hydroxymethyl)pyrrolidine-1-carboxamide; |
| 14 | (S)-N-(2-chloro-6-fluorophenyl)-5-fluoro-4-(3-methyl-3-propylureido)-2- |
| | ((1,1,1-trifluoropropan-2-yl)oxy)benzamide; |
| 15 | (S)-N-(2-chloro-6-fluorophenyl)-4-(3-ethyl-3-propylureido)-5-fluoro-2-((1,1,1- |
| | trifluoropropan-2-yl)oxy)benzamide; |
| 16 | (S)-N-(2-chloro-6-fluorophenyl)-5-fluoro-4-(3-(2-fluoroethyl)-3-methylureido)- |
| | 2-((1,1,1-trifluoropropan-2-yl)oxy)benzamide; |
| 17 | (S)-N-(2-chloro-6-fluorophenyl)-4-(3-(2-cyanoethyl)-3-methylureido)-5-fluoro- |
| | 2-((1,1,1-trifluoropropan-2-yl)oxy)benzamide; |
| 18 | (S)-N-(2-chloro-6-fluorophenyl)-5-fluoro-4-(3-(2-hydroxyethyl)-3- |
| | methylureido)-2-((1,1,1-trifluoropropan-2-yl)oxy)benzamide; |
| 19 | (S)-N-(4-((2-chloro-6-fluorophenyl)carbamoyl)-2-fluoro-5-((1,1,1- |
| | trifluoropropan-2-yl)oxy)phenyl)pyrrolidine-1-carboxamide; |
| 20 | (S)-N-(4-((2-chloro-6-fluorophenyl)carbamoyl)-2-fluoro-5-((1,1,1- |
| | trifluoropropan-2-yl)oxy)phenyl)azetidine-1-carboxamide; |
| 21 | N-(4-((2-chloro-6-fluorophenyl)carbamoyl)-2-fluoro-5-(((S)-1,1,1- |
| | trifluoropropan-2-yl)oxy)phenyl)-2-(hydroxymethyl)pyrrolidine-1-carboxamide; |
| 22 | (S)-N-(4-((2-chloro-6-fluorophenyl)carbamoyl)-2-fluoro-5-((1,1,1- |
| | trifluoropropan-2-yl)oxy)phenyl)-3-fluoroazetidine-1-carboxamide; |
| 23 | (S)-N-(4-((2-chloro-6-fluorophenyl)carbamoyl)-2-fluoro-5-((1,1,1- |
| | trifluoropropan-2-yl)oxy)phenyl)-3-hydroxyazetidine-1-carboxamide; |
| 24 | (S)-N-(4-((2-chloro-6-fluorophenyl)carbamoyl)-2-fluoro-5-((1,1,1- |
| | trifluoropropan-2-yl)oxy)phenyl)-3-(hydroxymethyl)azetidine-1-carboxamide; |
| 25 | (S)-N-(4-((2-chloro-6-fluorophenyl)carbamoyl)-2-fluoro-5-((1,1,1- |
| | trifluoropropan-2-yl)oxy)phenyl)-3-(fluoromethyl)azetidine-1-carboxamide; |
| 26 | (S)-N-(2-chloro-6-fluorophenyl)-4-(3-(cyanomethyl)-3-methylureido)-5-fluoro- |
| | 2-((1,1,1-trifluoropropan-2-yl)oxy)benzamide; |
| 27 | (R)-N-(4-((2-chloro-6-fluorophenyl)carbamoyl)-2-fluoro-5-(((S)-1,1,1- |
| | trifluoropropan-2-yl)oxy)phenyl)-2-(hydroxymethyl)pyrrolidine-1-carboxamide; |
| 28 | (S)-N-(4-((2-chloro-6-fluorophenyl)carbamoyl)-2-fluoro-5-(((S)-1,1,1- |
| | trifluoropropan-2-yl)oxy)phenyl)-2-(hydroxymethyl)pyrrolidine-1-carboxamide; |
| 29 | N-(4-((2-chloro-6-fluorophenyl)carbamoyl)-2-fluoro-5-(((S)-1,1,1- |
| | trifluoropropan-2-yl)oxy)phenyl)-3-methoxypyrrolidine-1-carboxamide; |
| 30 | N-(4-((2-chloro-6-fluorophenyl)carbamoyl)-2-fluoro-5-(((S)-1,1,1- |
| | trifluoropropan-2-yl)oxy)phenyl)-3-hydroxypyrrolidine-1-carboxamide; |
| 31 | N-(4-((2-chloro-6-fluorophenyl)carbamoyl)-2-fluoro-5-(((S)-1,1,1- |
| | trifluoropropan-2-yl)oxy)phenyl)-2-(methoxymethyl)pyrrolidine-1-carboxamide; |
| 32 | N-(4-((2-chloro-6-fluorophenyl)carbamoyl)-2-fluoro-5-(((S)-1,1,1- |
| | trifluoropropan-2-yl)oxy)phenyl)-3-fluoropyrrolidine-1-carboxamide; |
| 33 | (S)-N-(4-((2-chloro-6-fluorophenyl)carbamoyl)-2-fluoro-5-((1,1,1- |
| | trifluoropropan-2-yl)oxy)phenyl)-3,3-difluoropyrrolidine-1-carboxamide; |
| 34 | N-(4-((2-chloro-6-fluorophenyl)carbamoyl)-2-fluoro-5-(((S)-1,1,1- |
| | trifluoropropan-2-yl)oxy)phenyl)-2-(hydroxymethyl)-5-oxopyrrolidine-1- |
| | carboxamide; |
| 35 | (S)-N-(2-chloro-6-fluorophenyl)-4-(3,3-diethyl-1-methylureido)-5-fluoro-2- |
| | ((1,1,1-trifluoropropan-2-yl)oxy)benzamide; |

A further embodiment of the current invention is a compound as shown below in Table 2, or pharmaceutically acceptable salts, isotopes, tautomers, N-oxides, solvates, or stereoisomers thereof.

TABLE 2

| Example # | Compound Name |
|---|---|
| 1 | (S)-N-(2-chloro-6-fluorophenyl)-4-(3,3-diethylureido)-5-fluoro-2-((1,1,1- |
| | trifluoropropan-2-yl)oxy)benzamide; |
| 2 | (S)-N-(4-((2-chloro-6-fluorophenyl)carbamoyl)-2-fluoro-5-((1,1,1- |
| | trifluoropropan-2-yl)oxy)phenyl)piperidine-1-carboxamide; |
| 3 | (S)-N-(2-chloro-6-fluorophenyl)-4-(3-ethyl-3-(2-hydroxyethyl)ureido)-5-fluoro- |
| | 2-((1,1,1-trifluoropropan-2-yl)oxy)benzamide; |
| 4 | (S)-N-(2-chloro-6-fluorophenyl)-4-(3-ethyl-3-isopropylureido)-5-fluoro-2- |
| | ((1,1,1-trifluoropropan-2-yl)oxy)benzamide; |
| 5 | (S)-N-(2-chloro-6-fluorophenyl)-4-(3-cyclopropyl-3-ethylureido)-5-fluoro-2- |
| | ((1,1,1-trifluoropropan-2-yl)oxy)benzamide; |
| 6 | (S)-N-(2-chloro-6-fluorophenyl)-4-(3-ethyl-3-(2-methoxyethyl)ureido)-5-fluoro- |
| | 2-((1,1,1-trifluoropropan-2-yl)oxy)benzamide; |
| 7 | (S)-N-(2-chloro-6-fluorophenyl)-4-(3-(2,2-difluoroethyl)-3-ethylureido)-5- |
| | fluoro-2-((1,1,1-trifluoropropan-2-yl)oxy)benzamide; |
| 8 | (S)-N-(2-chloro-6-fluorophenyl)-4-(3-ethylureido)-5-fluoro-2-((1,1,1- |
| | trifluoropropan-2-yl)oxy)benzamide; |
| 9 | (S)-N-(2-chloro-6-fluorophenyl)-5-fluoro-4-(3-isopropylureido)-2-((1,1,1- |
| | trifluoropropan-2-yl)oxy)benzamide; |
| 10 | (S)-N-(2-chloro-6-fluorophenyl)-4-(3,3-dimethylureido)-5-fluoro-2-((1,1,1- |
| | trifluoropropan-2-yl)oxy)benzamide; |
| 11 | (S)-N-(2-chloro-6-fluorophenyl)-4-(3-ethyl-3-methylureido)-5-fluoro-2-((1,1,1- |
| | trifluoropropan-2-yl)oxy)benzamide; |
| 12 | N-(4-((2-chloro-6-fluorophenyl)carbamoyl)-2-fluoro-5-(((S)-1,1,1- |
| | trifluoropropan-2-yl)oxy)phenyl)-2-(hydroxymethyl)azetidine-1-carboxamide; |
| 13 | N-(4-((2-chloro-6-fluorophenyl)carbamoyl)-2-fluoro-5-(((S)-1,1,1- |
| | trifluoropropan-2-yl)oxy)phenyl)-3-(hydroxymethyl)pyrrolidine-1-carboxamide; |
| 14 | (S)-N-(2-chloro-6-fluorophenyl)-5-fluoro-4-(3-methyl-3-propylureido)-2- |
| | ((1,1,1-trifluoropropan-2-yl)oxy)benzamide; |
| 15 | (S)-N-(2-chloro-6-fluorophenyl)-4-(3-ethyl-3-propylureido)-5-fluoro-2-((1,1,1- |
| | trifluoropropan-2-yl)oxy)benzamide; |
| 16 | (S)-N-(2-chloro-6-fluorophenyl)-5-fluoro-4-(3-(2-fluoroethyl)-3-methylureido)- |
| | 2-((1,1,1-trifluoropropan-2-yl)oxy)benzamide; |
| 17 | (S)-N-(2-chloro-6-fluorophenyl)-4-(3-(2-cyanoethyl)-3-methylureido)-5-fluoro- |
| | 2-((1,1,1-trifluoropropan-2-yl)oxy)benzamide; |
| 18 | (S)-N-(2-chloro-6-fluorophenyl)-5-fluoro-4-(3-(2-hydroxyethyl)-3- |
| | methylureido)-2-((1,1,1-trifluoropropan-2-yl)oxy)benzamide; |
| 19 | (S)-N-(4-((2-chloro-6-fluorophenyl)carbamoyl)-2-fluoro-5-((1,1,1- |
| | trifluoropropan-2-yl)oxy)phenyl)pyrrolidine-1-carboxamide; |
| 20 | (S)-N-(4-((2-chloro-6-fluorophenyl)carbamoyl)-2-fluoro-5-((1,1,1- |
| | trifluoropropan-2-yl)oxy)phenyl)azetidine-1-carboxamide; |
| 21 | N-(4-((2-chloro-6-fluorophenyl)carbamoyl)-2-fluoro-5-(((S)-1,1,1- |
| | trifluoropropan-2-yl)oxy)phenyl)-2-(hydroxymethyl)pyrrolidine-1-carboxamide; |
| 22 | (S)-N-(4-((2-chloro-6-fluorophenyl)carbamoyl)-2-fluoro-5-((1,1,1- |
| | trifluoropropan-2-yl)oxy)phenyl)-3-fluoroazetidine-1-carboxamide; |
| 23 | (S)-N-(4-((2-chloro-6-fluorophenyl)carbamoyl)-2-fluoro-5-((1,1,1- |
| | trifluoropropan-2-yl)oxy)phenyl)-3-hydroxyazetidine-1-carboxamide; |
| 24 | (S)-N-(4-((2-chloro-6-fluorophenyl)carbamoyl)-2-fluoro-5-((1,1,1- |
| | trifluoropropan-2-yl)oxy)phenyl)-3-(hydroxymethyl)azetidine-1-carboxamide; |
| 25 | (S)-N-(4-((2-chloro-6-fluorophenyl)carbamoyl)-2-fluoro-5-((1,1,1- |
| | trifluoropropan-2-yl)oxy)phenyl)-3-(fluoromethyl)azetidine-1-carboxamide; |
| 26 | (S)-N-(2-chloro-6-fluorophenyl)-4-(3-(cyanomethyl)-3-methylureido)-5-fluoro- |
| | 2-((1,1,1-trifluoropropan-2-yl)oxy)benzamide; |
| 27 | (R)-N-(4-((2-chloro-6-fluorophenyl)carbamoyl)-2-fluoro-5-(((S)-1,1,1- |
| | trifluoropropan-2-yl)oxy)phenyl)-2-(hydroxymethyl)pyrrolidine-1-carboxamide; |
| 28 | (S)-N-(4-((2-chloro-6-fluorophenyl)carbamoyl)-2-fluoro-5-(((S)-1,1,1- |
| | trifluoropropan-2-yl)oxy)phenyl)-2-(hydroxymethyl)pyrrolidine-1-carboxamide; |
| 29 | N-(4-((2-chloro-6-fluorophenyl)carbamoyl)-2-fluoro-5-(((S)-1,1,1- |
| | trifluoropropan-2-yl)oxy)phenyl)-3-methoxypyrrolidine-1-carboxamide; |
| 30 | N-(4-((2-chloro-6-fluorophenyl)carbamoyl)-2-fluoro-5-(((S)-1,1,1- |
| | trifluoropropan-2-yl)oxy)phenyl)-3-hydroxypyrrolidine-1-carboxamide; |
| 31 | N-(4-((2-chloro-6-fluorophenyl)carbamoyl)-2-fluoro-5-(((S)-1,1,1- |
| | trifluoropropan-2-yl)oxy)phenyl)-2-(methoxymethyl)pyrrolidine-1-carboxamide; |
| 32 | N-(4-((2-chloro-6-fluorophenyl)carbamoyl)-2-fluoro-5-(((S)-1,1,1- |
| | trifluoropropan-2-yl)oxy)phenyl)-3-fluoropyrrolidine-1-carboxamide; |
| 33 | (S)-N-(4-((2-chloro-6-fluorophenyl)carbamoyl)-2-fluoro-5-((1,1,1- |
| | trifluoropropan-2-yl)oxy)phenyl)-3,3-difluoropyrrolidine-1-carboxamide; |
| 34 | N-(4-((2-chloro-6-fluorophenyl)carbamoyl)-2-fluoro-5-(((S)-1,1,1- |
| | trifluoropropan-2-yl)oxy)phenyl)-2-(hydroxymethyl)-5-oxopyrrolidine-1- |
| | carboxamide; |
| 35 | (S)-N-(2-chloro-6-fluorophenyl)-4-(3,3-diethyl-1-methylureido)-5-fluoro-2- |
| | ((1,1,1-trifluoropropan-2-yl)oxy)benzamide; |
| 36 | (R)-N-(4-((2-Chloro-4-methylpyridin-3-yl)carbamoyl)-2-fluoro-5-(((S)-1,1,1- |
| | trifluoropropan-2-yl)oxy)phenyl)-2-(hydroxymethyl)pyrrolidine-1-carboxamide; |
| 37 | (R)-N-(4-((5-Chloro-3-methyl-1H-pyrazol-4-yl)carbamoyl)-2-fluoro-5-(((S)- |
| | 1,1,1-trifluoropropan-2-yl)oxy)phenyl)-2-(hydroxymethyl)pyrrolidine-1- |
| | carboxamide; |
| 38 | (R)-N-(2-Fluoro-4-((2-methoxy-4-methylpyridin-3-yl)carbamoyl)-5-(((S)-1,1,1- |
| | trifluoropropan-2-yl)oxy)phenyl)-2-(hydroxymethyl)pyrrolidine-1-carboxamide; |
| 39 | (R)-N-(4-((3-Chloro-2-methoxy-5-methylpyridin-4-yl)carbamoyl)-2-fluoro-5- |
| | (((S)-1,1,1-trifluoropropan-2-yl)oxy)phenyl)-2-(hydroxymethyl)pyrrolidine-1- |
| | carboxamide; |
| 40 | (R)-N-(2-Fluoro-4-((2-methoxy-3,5-dimethylpyridin-4-yl)carbamoyl)-5-(((S)- |
| | 1,1,1-trifluoropropan-2-yl)oxy)phenyl)-2-(hydroxymethyl)pyrrolidine-1- |
| | carboxamide; |
| 41 | (S)-N-(2-Chloro-4-methylpyridin-3-yl)-4-(3-(2,2-difluoroethyl)-3-ethylureido)- |
| | 5-fluoro-2-((1,1,1-trifluoropropan-2-yl)oxy)benzamide; |
| 42 | (S)-N-(5-Chloro-3-methyl-1H-pyrazol-4-yl)-4-(3-(2,2-difluoroethyl)-3- |
| | ethylureido)-5-fluoro-2-((1,1,1-trifluoropropan-2-yl)oxy)benzamide; |
| 43 | (S)-4-(3-(2,2-difluoroethyl)-3-ethylureido)-5-fluoro-N-(2-methoxy-4- |
| | methylpyridin-3-yl)-2-((1,1,1-trifluoropropan-2-yl)oxy)benzamide; |
| 44 | (S)-N-(3-Chloro-2-methoxy-5-methylpyridin-4-yl)-4-(3-(2,2-difluoroethyl)-3- |
| | ethylureido)-5-fluoro-2-((1,1,1-trifluoropropan-2-yl)oxy)benzamide; |
| 45 | (S)-4-(3-(2,2-Difluoroethyl)-3-ethylureido)-5-fluoro-N-(2-methoxy-3,5- |
| | dimethylpyridin-4-yl)-2-((1,1,1-trifluoropropan-2-yl)oxy)benzamide; |

Also within the scope of the invention are enantiomers and diastereomers of the compounds of Formulas (I), (II) and (III). Also within the scope of the invention are the pharmaceutically acceptable salts, N-oxides or solvates of the compounds of Formulas (I), (II) and (III). Also within the scope of the invention are the pharmaceutically acceptable prodrugs of compounds of Formulas (I), (II) and (III), and pharmaceutically active metabolites of the compounds of Formulas (I), (II) and (III).

Also within the scope of the invention are isotopic variations of compounds of Formulas (I), (II) and (III), such as, e.g., deuterated compounds of Formulas (I), (II) and (III). Also within the scope of the invention are the pharmaceutically acceptable salts, N-oxides or solvates of the isotopic variations of the compounds of Formulas (I), (II) and (III). Also within the scope of the invention are the pharmaceutically acceptable prodrugs of the isotopic variations of the compounds of Formulas (I), (II) and (III), and pharmaceutically active metabolites of the isotopic variations of the compounds of Formulas (I), (II) and (III).

Even though the compounds of embodiments of the present invention (including their pharmaceutically acceptable salts and pharmaceutically acceptable solvates) can be administered alone, they will generally be administered in admixture with a pharmaceutically acceptable carrier, a pharmaceutically acceptable excipient and/or a pharmaceutically acceptable diluent selected with regard to the intended route of administration and standard pharmaceutical or veterinary practice.

Thus, particular embodiments of the present invention are directed to pharmaceutical and veterinary compositions comprising compounds of Formulas (I), (II) and (III) and at least one pharmaceutically acceptable carrier, pharmaceutically acceptable excipient, and/or pharmaceutically acceptable diluent. By way of example, in the pharmaceutical compositions of embodiments of the present invention, the compounds of Formulas (I), (II) and (III) may be admixed with any suitable binder(s), lubricant(s), suspending agent(s), coating agent(s), solubilizing agent(s), and combinations thereof.

An embodiment of the invention relates to a pharmaceutical composition comprising an effective amount of at least one compound selected from compounds of Formulas (I), (II) and (III), and pharmaceutically acceptable salts, isotopes, N-oxides, solvates, and stereoisomers thereof, in accordance with any embodiment described herein; and at least one pharmaceutically acceptable excipient.

Another embodiment of the invention is a pharmaceutical composition comprising an effective amount of at least one compound selected from compounds of Formulas (I), (II) and (III).

An additional embodiment of the invention is a pharmaceutical composition comprising an effective amount of a compound shown in Table 1 (e.g., a compound selected from Examples 1-35), or a pharmaceutically acceptable salt, isotope, N-oxide, solvate, or stereoisomer of the compound of Table 1, a pharmaceutically acceptable prodrug of the compound of Table 1, or a pharmaceutically active metabolite of the compound of Table 1; and at least one pharmaceutically acceptable excipient.

An additional embodiment of the invention is a pharmaceutical composition comprising an effective amount of a compound shown in Table 2 (e.g., a compound selected from Examples 1-45), or a pharmaceutically acceptable salt, isotope, N-oxide, solvate, or stereoisomer of the compound of Table 2, a pharmaceutically acceptable prodrug of the compound of Table 2, or a pharmaceutically active metabolite of the compound of Table 2; and at least one pharmaceutically acceptable excipient.

Solid oral dosage forms such as, tablets or capsules, containing one or more compounds of the present invention may be administered in at least one dosage form at a time, as appropriate. It is also possible to administer the compounds in sustained release formulations.

Additional oral forms in which the present inventive compounds may be administered include elixirs, solutions, syrups, and suspensions; each optionally containing flavoring agents and coloring agents.

Alternatively, one or more compounds of Formulas (I), (II) and (III) can be administered by inhalation (intratracheal or intranasal) or in the form of a suppository or pessary, or they may be applied topically in the form of a lotion, solution, cream, ointment or dusting powder. For example, they can be incorporated into a cream comprising, consisting of, and/or consisting essentially of an aqueous emulsion of polyethylene glycols or liquid paraffin. They can also be incorporated, at a concentration of between about 1% and about 10% by weight of the cream, into an ointment comprising, consisting of, and/or consisting essentially of a wax or soft paraffin base together with any stabilizers and preservatives as may be required. An alternative means of administration includes transdermal administration by using a skin or transdermal patch.

The pharmaceutical compositions of the present invention (as well as the compounds of the present invention alone) can also be injected parenterally, for example, intracavernosally, intravenously, intramuscularly, subcutaneously, intradermally, or intrathecally. In this case, the compositions will also include at least one of a suitable carrier, a suitable excipient, and a suitable diluent.

For parenteral administration, the pharmaceutical compositions of the present invention are best used in the form of a sterile aqueous solution that may contain other substances, for example, enough salts and monosaccharides to make the solution isotonic with blood.

For buccal or sublingual administration, the pharmaceutical compositions of the present invention may be administered in the form of tablets or lozenges, which can be formulated in a conventional manner.

By way of further example, pharmaceutical compositions containing at least one of the compounds of Formula (I), (II) or (III) as the active ingredient can be prepared by mixing the compound(s) with a pharmaceutically acceptable carrier, a pharmaceutically acceptable diluent, and/or a pharmaceutically acceptable excipient according to conventional pharmaceutical compounding techniques. The carrier, excipient, and diluent may take a wide variety of forms depending upon the desired route of administration (e.g., oral, parenteral, etc.). Thus, for liquid oral preparations such as, suspensions, syrups, elixirs and solutions, suitable carriers, excipients and diluents include water, glycols, oils, alcohols, flavoring agents, preservatives, stabilizers, coloring agents and the like; for solid oral preparations such as, powders, capsules, and tablets, suitable carriers, excipients and diluents include starches, sugars, diluents, granulating agents, lubricants, binders, disintegrating agents and the like. Solid oral preparations also may be optionally coated with substances such as, sugars, or be enterically coated so as to modulate the major site of absorption and disintegration. For parenteral administration, the carrier, excipient and diluent will usually include sterile water, and other ingredients may be added to increase solubility and preservation of the composition. Injectable suspensions or solutions may also be prepared utilizing aqueous carriers along with appropriate additives such as, solubilizers and preservatives.

According to particular embodiments, a therapeutically effective amount of a compound of Formula (I), (II) or (III) or a pharmaceutical composition thereof may comprise a dose range from about 0.1 mg to about 3000 mg, or any particular amount or range therein, in particular from about 1 mg to about 1000 mg, or any particular amount or range therein, of active ingredient in a regimen of about 1 to about (4×) per day for an average (70 kg) human; although, it is apparent to one skilled in the art that the therapeutically effective amount for a compound of Formula (I) will vary as will the diseases, syndromes, conditions, and disorders being treated.

An embodiment of the present invention is directed to a pharmaceutical composition for oral administration, comprising a compound of Formula (I), (II) or (III) in an amount of from about 1 mg to about 500 mg.

Advantageously, a compound of Formula (I), (II) or (III) may be administered in a single daily dose, or the total daily dosage may be administered in divided doses of two, three and (4×) daily.

Optimal dosages of a compound of Formula (I), (II) or (III) to be administered may be readily determined and will vary with the particular compound used, the mode of administration, the strength of the preparation, and the advancement of the disease, syndrome, condition or disorder. In addition, factors associated with the particular subject being treated, including subject gender, age, weight, diet and time of administration, will result in the need to adjust the dose to achieve an appropriate therapeutic level and desired therapeutic effect. The above dosages are thus exemplary of the average case. There can be, of course, individual instances wherein higher or lower dosage ranges are merited, and such are within the scope of this invention.

Compounds of Formula (I), (II) or (III) may be administered in any of the foregoing compositions and dosage regimens or by means of those compositions and dosage regimens established in the art whenever use of a compound of Formula (I), (II) or (III) is administered to a subject in need thereof.

According to particular embodiments, one or more compounds of Formula (I), (II) or (III) are useful in methods for treating, ameliorating and/or preventing a disease, a syndrome, a condition or a disorder that is affected by the inhibition of DHODH enzymatic activity.

An additional embodiment of the invention relates to the use of compounds of Formula (I), (II) or (III), e.g., by inhibiting dihydroorotate oxygenase enzyme activity, in treating disorders like inflammatory disorders, autoimmune disorders, or cancer.

In a further aspect the present invention provides a method for inhibiting or altering Dihydroorotate Dehydrogenase (DHODH) enzymatic activity, the method comprising contacting DHODH with any compound of Formula (I), (II) or (III), aspect or embodiment disclosed herein, thereby inhibiting or otherwise altering DHODH enzymatic activity.

An additional embodiment of the present invention provides methods for treating diseases, disorders, or medical conditions mediated or otherwise affected by dihydroorotate dehydrogenase (DHODH) enzyme activity comprising administering a compound of Formula (I), (II) or (III) to a subject in need thereof.

As used herein, the term “DHODH inhibitor” may refer to an agent that inhibits or reduces DHODH activity.

In one embodiment, the term “therapeutically effective amount” (or “effective amount”) refers to the amount of a compound of the present invention that, when administered to a subject, is effective to (1) at least partially alleviate, inhibit, prevent, and/or ameliorate a condition, or a disorder or a disease (i) mediated by DHODH enzymatic activity; or (ii) associated with DHODH enzymatic activity; or (iii) characterized by activity (normal or abnormal) of DHODH enzyme; or (2) reduce or inhibit the activity of DHODH enzyme; or (3) reduce or inhibit the expression of DHODH; or (4) modify the protein levels of DHODH. Without being bound by a particular theory, DHODH inhibitors are believed to act by inhibiting nucleic acid synthesis, cell cycle arrest or altering post-translational glycosylation of proteins involved in regulating myeloid differentiation within progenitor tumor cells.

An additional embodiment of the invention is a method of treating a subject suffering from or diagnosed with a disease, disorder, or medical condition mediated or otherwise affected by DHODH enzymatic activity, comprising administering to a subject in need of such treatment an effective amount of at least one compound selected from: compounds of Formula (I), (II) or (III), enantiomers and diastereomers of the compounds of Formula (I), (II) or (III), isotopic variations of the compounds of Formula (I), (II) or (III), and pharmaceutically acceptable salts of all of the foregoing. Stated another way, according to an embodiment, a method of treating a subject suffering from or diagnosed with a disease, disorder, or medical condition, such as cancer, comprises administering to the subject an effective amount of at least one compound selected from: compounds of Formula (I), (II) or (III), and pharmaceutically acceptable salts of all the foregoing (e.g., by inhibiting or otherwise altering dihydroorotate oxygenase enzyme activity in the subject).

In another embodiment, inhibitors of DHODH of the present invention may be used for the treatment of immunological diseases including, but not limited to, autoimmune and inflammatory disorders, e.g. arthritis, inflammatory bowel disease, gastritis, ankylosing spondylitis, ulcerative colitis, pancreatitis, Crohn's disease, celiac disease, multiple sclerosis, systemic lupus erythematosus, lupus nephritis, rheumatic fever, gout, organ or transplant rejection, chronic allograft rejection, acute or chronic graft-versus-host disease, dermatitis including atopic, dermatomyositis, psoriasis, Behcet's diseases, uveitis, myasthenia gravis, Grave's disease, Hashimoto thyroiditis, Sjogren's syndrome, blistering disorders, antibody-mediated vasculitis syndromes, immune-complex vasculitides, allergic disorders, asthma, bronchitis, chronic obstructive pulmonary disease (COPD), cystic fibrosis, pneumonia, pulmonary diseases including edema, embolism, fibrosis, sarcoidosis, hypertension and emphysema, silicosis, respiratory failure, acute respiratory distress syndrome, BENTA disease, berylliosis, and polymyositis.

As used herein, unless otherwise noted, the term “affect” or “affected” (when referring to a disease, disorder, or medical condition that is affected by the inhibition or alteration of DHODH enzymatic activity) includes a reduction in the frequency and/or severity of one or more symptoms or manifestations of said disease, syndrome, condition or disorder; and/or includes the prevention of the development of one or more symptoms or manifestations of said disease, syndrome, condition or disorder or the development of the disease, condition, syndrome or disorder.

An additional embodiment of the invention provides a method of treatment of cancer comprising administering to a subject in need thereof, a therapeutically effective amount of a compound of Formula (I), or a pharmaceutically acceptable salt, isotope, N-oxide, solvate, or stereoisomer thereof.

According to an embodiment, the cancer is selected from but not limited to, lymphomas, leukemias, carcinomas, and sarcomas.

An additional embodiment of the invention provides the use of a compound of Formula (I), (II) or (III), or a pharmaceutically acceptable salt, isotope, N-oxide, solvate, or stereoisomer thereof, for the treatment of one or more cancer types.

According to particular embodiments, the uses and methods of treatment described herein are directed to the treatment of cancer, wherein the cancer is selected from but not limited to:
leukemias including but not limited to acute lymphoblastic leukemia (ALL), acute myeloid leukemia (AML), (acute) T-cell leukemia, acute monocytic leukemia, acute promyelocytic leukemia (APL), bisphenotypic B myelomonocytic leukemia, chronic myeloid leukemia (CML), chronic myelomonocytic leukemia (CMML), large granular lymphocytic leukemia, plasma cell leukemia, and also myelodysplastic syndrome (MDS), which can develop into an acute myeloid leukemia,
lymphomas including but not limited to AIDS-related lymphoma, Hodgkin lymphoma, non-Hodgkin's lymphoma (NHL), T-non-Hodgkin lymphoma (T-NHL), subtypes of NHL such as Diffuse Large Cell Lymphoma (DLBCL), activated B-cell DLBCL, germinal center B-cell DLBCL, double-hit lymphoma and double-expressor lymphoma; anaplastic large cell lymphoma, marginal B cell lymphoma and primary mediastinal B-cell lymphoma, immunoblastic large cell lymphoma, Burkitt lymphoma, follicular lymphoma, hairy cell leukemia, Hodgkin's disease, mantle cell lymphoma (MCL), lymphoplasmatic lymphoma, precursor B-lymphoblastic lymphoma, lymphoma of the central nervous system, small lymphocytic lymphoma (SLL) and chronic lymphocytic leukemia (CLL); T-cell NHL such as precursor T-lymphoblastic lymphoma/leukemia, peripheral T-cell lymphoma (PTCL), cutaneous T-cell lymphoma (CTCL), angioimmunoblastic T-cell lymphoma, extranodal natural killer T-cell lymphoma, enteropathy type T-cell lymphoma, subcutaneous panniculitis-like T-cell lymphoma, anaplastic large cell lymphoma
sarcomas including but not limited to sarcoma of the soft tissue, gliosarcoma, osteosarcoma, malignant fibrous histiocytoma, lymphosarcoma, and rhabdomyosarcoma;
   and
other cancers, such as solid tumors, including but not limited to breast cancer, colorectal carcinoma, gastric cancer, gliosarcoma, head & neck cancer, hepatocellular carcinoma, lung cancer, multiple myeloma, neuroblastoma, ovarian cancer, pancreatic cancer, prostate cancer, renal cell carcinoma and sarcoma.

In an embodiment, cancers that may benefit from a treatment with inhibitors of DHODH of the present invention include, but are not limited to, lymphomas, leukemias, carcinomas, and sarcomas, e.g. non-Hodgkin's lymphoma, diffuse large B-cell lymphoma (DLBCL), mantle cell lymphoma (MCL), follicular lymphoma (FL), marginal zone lymphoma, T-cell lymphoma, Hodgkin's lymphoma, Burkitt's lymphoma, multiple myeloma, brain (gliomas), glioblastomas, breast cancer, colorectal/colon cancer, prostate cancer, lung cancer including non-small-cell, gastric cancer, endometrial cancer, melanoma, pancreatic cancer, liver cancer, kidney cancer, squamous cell carcinoma, ovarian cancer, sarcoma, osteosarcoma, thyroid cancer, bladder cancer, head & neck cancer, testicular cancer, Ewing's sarcoma, rhabdomyosarcoma, medulloblastoma, neuroblastoma, cervical cancer, renal cancer, urothelial cancer, vulval cancer, esophageal cancer, salivary gland cancer, nasopharangeal cancer, buccal cancer, cancer of the mouth, and GIST (gastrointestinal stromal tumor).

In another embodiment of the present invention, the compounds of the present invention may be employed in combination with one or more other medicinal agents, more particularly with one or more anti-cancer agents, e.g. chemotherapeutic, anti-proliferative or immunomodulating agents, or with adjuvants in cancer therapy, e.g. immunosuppressive or anti-inflammatory agents. Additional non-limiting examples of anti-cancer agents that may be administered in combination with a compound of the present invention include biologic compounds, such as monoclonal antibodies (e.g., that mediate effector function upon binding to cancer cell-associated antigens, or block interaction of a receptor expressed on cancer cells with a soluble or cell bound ligand), bispecific antibodies that mediate immune cell redirection, etc. According to an embodiment, a method of treating cancer comprises administering an effective amount of a compound of the present invention (e.g., selected from compounds of Formula (I), (II) or (III), such as a compound shown in Table 1 or Table 2, pharmaceutically acceptable salts, isotopes, N-oxides, solvates, and stereoisomers thereof) and an effective amount of one or more additional anti-cancer agents, wherein the method comprises administering the compound of the present invention and the additional anti-cancer agent(s) either simultaneously (e.g., as part of the same pharmaceutical composition) or sequentially. According to an embodiment, a pharmaceutical composition comprises an effective amount of a compound of the present invention (e.g., selected from compounds of Formula (I), (II) or (III), such as a compound shown in Table 1 or Table 2, pharmaceutically acceptable salts, isotopes, N-oxides, solvates, and stereoisomers thereof), an effective amount of one or more additional anti-cancer agents, and optionally one or more excipients.

An additional embodiment of the invention provides the use of a compound of Formula (I), (II) or (III), or pharmaceutically acceptable salts, isotopes, N-oxides, solvates, or stereoisomers thereof, as part of chemotherapeutic regimens for the treatment of cancers, lymphomas and leukemias alone or in combination with classic antitumoral compounds well known by the one skilled in the art.

### General Synthetic Methods

Exemplary compounds useful in methods of the invention will now be described by reference to the illustrative synthetic schemes for their general preparation below and the specific examples that follow. Artisans will recognize that, to obtain the various compounds herein, starting materials may be suitably selected so that the ultimately desired substituents will be carried through the reaction scheme with or without protection as appropriate to yield the desired product. Alternatively, it may be necessary or desirable to employ, in the place of the ultimately desired substituent, a suitable group that may be carried through the reaction scheme and replaced as appropriate with the desired substituent. Unless otherwise specified, the variables are as defined above in reference to Formula (I). Reactions may be performed between the melting point and the reflux temperature of the solvent, and preferably between 0° C. and the reflux temperature of the solvent. Reactions may be heated employing conventional heating or microwave heating. Reactions may also be conducted in sealed pressure vessels above the normal reflux temperature of the solvent.

Abbreviations used in the instant specification, particularly the schemes and examples, are as follows:

| | |
|---|---|
| ACN or MeCN | acetonitrile |
| aq. | Aqueous |
| BINAP | (2,2′-bis(diphenylphosphino)-1,1′-binaphthyl) |
| Boc₂O | di-tert-butyl pyrocarbonate |
| DAST | diethylaminosulfur trifluoride |
| DCE | 1,2-dichloroethane |
| DCM | dichloromethane |
| DMF | N,N-dimethylformamide |
| DMAP | dimethylaminopyridine |
| DMP | Dess-Martin periodinane; 3-oxo-1,3-dihydro-1λ⁵,2- |
| | benziodoxole-1,1,1-triyl triacetate |
| DMSO | dimethylsulfoxide |
| EtOAc | ethyl acetate |
| ESI | electrospray ionization |
| Et | ethyl |
| Et₂O | diethylether |
| EtOH | ethanol |
| Et₃N | triethylamine |
| h or hr(s) | hour or hours |
| HPLC | high performance liquid chromatography |
| Me | methyl |
| MeOH | methanol |
| MHz | megahertz |
| min | minute or minutes |
| MS | mass spectrometry |
| NMR | nuclear magnetic resonance |
| NMO | N-methylmorpholine-N-oxide |
| OAc | acetate |
| Pd₂(dba)₃ | Tris(dibenzylideneacetone)dipalladium(0) |
| RP | reverse-phase |
| rt or RT | room temperature |
| Rₜ | retention time |
| Sec | second or seconds |
| TBAF | tetrabutylammonium fluoride |
| TBDPSCl | tert-butyldiphenylsilyl chloride |
| TBS | tert-butyldimethylsilyl |
| TBSCl | tert-butyldimethylsilyl chloride |
| TEA | triethylamine |
| TFA | trifluoroacetic acid |
| THF | tetrahydrofuran |
| TLC | thin layer chromatography |
| T₃P | propylphosphonic anhydride |
| Xantphos | 4,5-bis(diphenylphosphino)-9,9-dimethylxanthene |

### Preparative Examples

Exemplary compounds useful in methods of the invention will now be described by reference to the illustrative synthetic schemes for their general preparation below and the specific examples to follow.

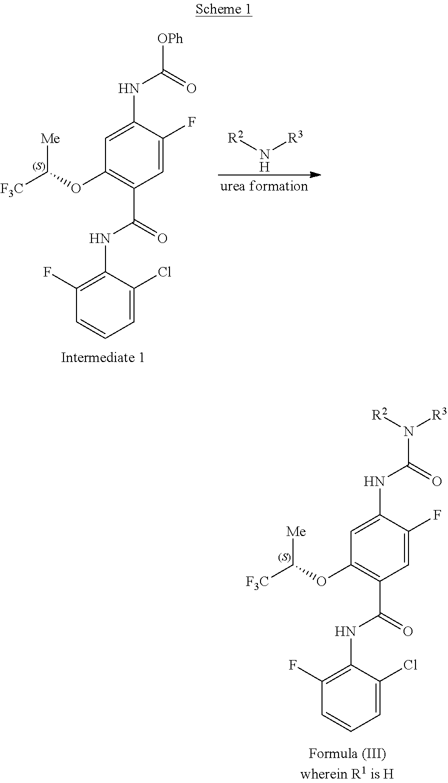

Compounds of Formula (III), wherein R¹ is H and R² and R³ are as defined in Formula (III), may be made as shown in Scheme 1. (S)-Phenyl (4-((2-chloro-6-fluorophenyl)carbamoyl)-2-fluoro-5-((1,1,1-trifluoropropan-2-yl)oxy)phenyl)carbamate, prepared as described in Intermediate 1—Step F, is reacted with a secondary amine in a polar aprotic solvent such as DCM and the like, in the presence of a suitable base such as Et₃N, NaH and the like, at ambient temperature.

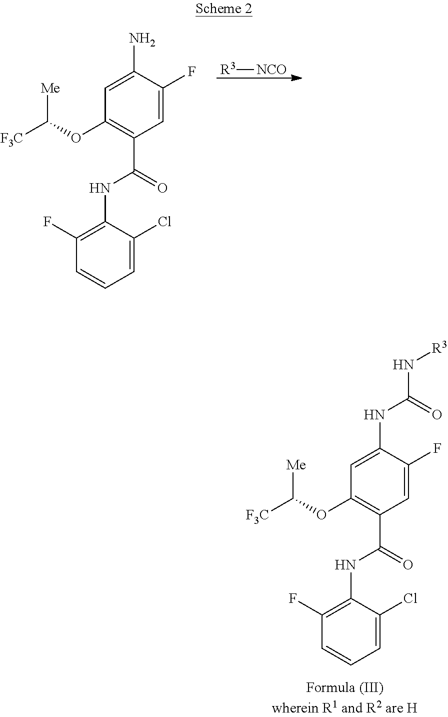

Compounds of Formula (III), wherein R¹ and R² are H and R³ is as defined in Formula (III), may be made as shown in Scheme 2. (S)-4-amino-N-(2-chloro-6-fluorophenyl)-5-fluoro-2-((1,1,1-trifluoropropan-2-yl)oxy)benzamide, prepared as described in Intermediate 1—Step E, is treated with an isocyanate of Formula R³—NCO, where R³ is defined as described in Formula (III), in a polar aprotic solvent such as DCM and the like, with a base such as Et₃N and the like, at elevated temperatures such as 100° C.

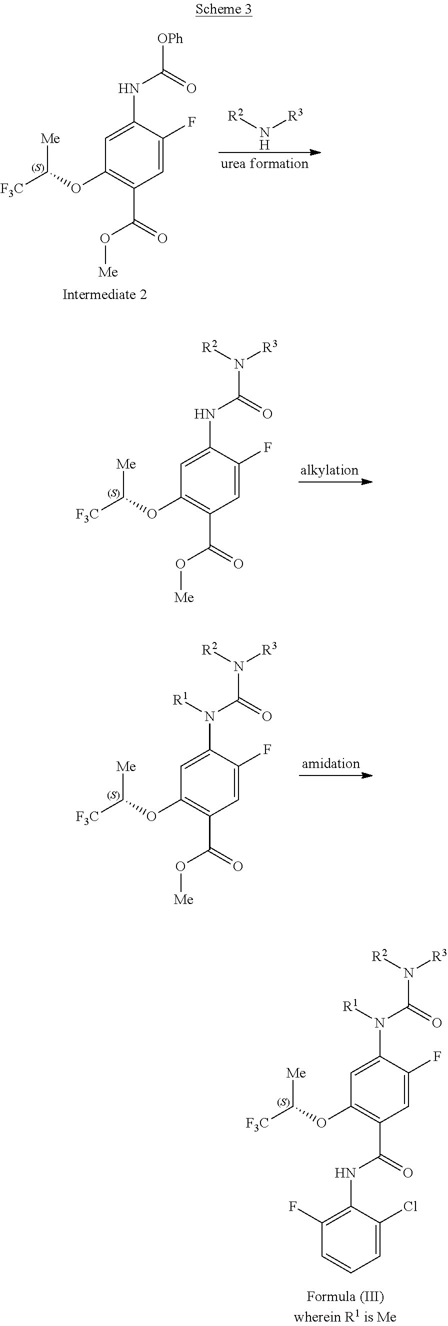

Compounds of Formula (III), wherein R¹ is Me and R² and R³ are as defined in Formula (III), may be made as shown in Scheme 3. Methyl (S)-5-fluoro-4-((phenoxycarbonyl)amino)-2-((1,1,1-trifluoropropan-2-yl)oxy)benzoate, prepared as described in Intermediate 2 is reacted with a secondary amine in a polar aprotic solvent such as DCM and the like, in the presence of a suitable base such as Et₃N, NaH and the like, at ambient temperature. Methylation can then be achieved using an alkylating agent such as Mel, and the like, in a polar aprotic solvent such as THF, DMF and the like, and employing a suitable base such as NaH and the like. Treatment of the ester under amide bond forming conditions such as AlMe₃, with 2-chloro-6-fluoroaniline in a solvent such as DCM and the like, at elevated temperatures, preferably 60° C., generates a compound of Formula (III).

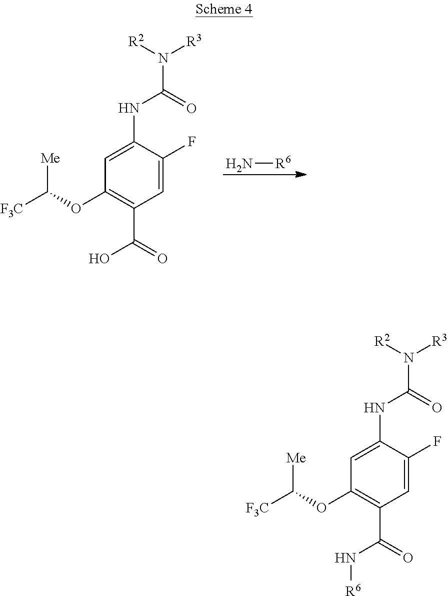

Compounds of Formula (I), wherein R², R³, and R⁶ are as defined above, may be prepared as shown in Scheme 4 by conversion of the corresponding carboxylic acid using methods known to one skilled in the art. For example, converting the acid to the acid chloride using POCl₃, and treating with a commercially available or synthetically accessible amine provides a compound of Formula (I). In an alternative method, an amide coupling reagent, such as T₃P, along with a suitable base, such as Et₃N, can be employed. The starting material for Scheme 4 may be prepared by saponification of the corresponding methyl ester, which may be prepared as shown in Scheme 3.

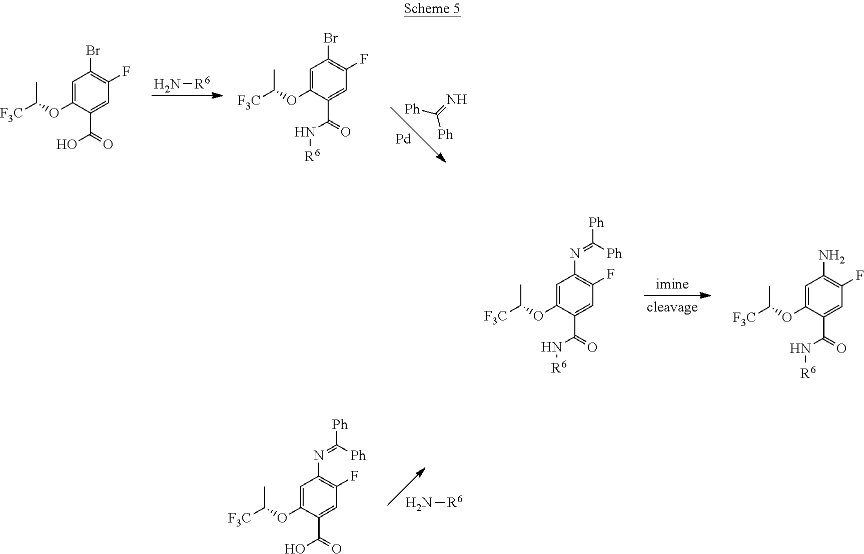

According to Scheme 5, (S)-4-bromo-5-fluoro-2-((1,1,1-trifluoropropan-2-yl)oxy)benzoic acid, as prepared in Intermediate 1—Step B, is reacted with a commercially available or synthetically accessible amine R⁶—NH₂ using conditions known to one skilled in the art. A preferred method uses POCl₃ and an appropriate base such as pyridine in DCM. A subsequent amination reaction using benzophenone imine, a palladium catalyst, such as Pd₂(dba)₃, a ligand such as Xantphos, a base such as Cs₂CO₃, in a suitable solvent at elevated temperatures provides the imine product. Alternatively, (S)-4-((diphenylmethylene)amino)-5-fluoro-2-((1,1,1-trifluoropropan-2-yl)oxy)benzoic acid is reacted with a commercially available or synthetically accessible amine R⁶—NH₂ using conditions described above. Subsequent imine cleavage under acidic conditions such as HCl in MeOH, affords the aniline intermediate.

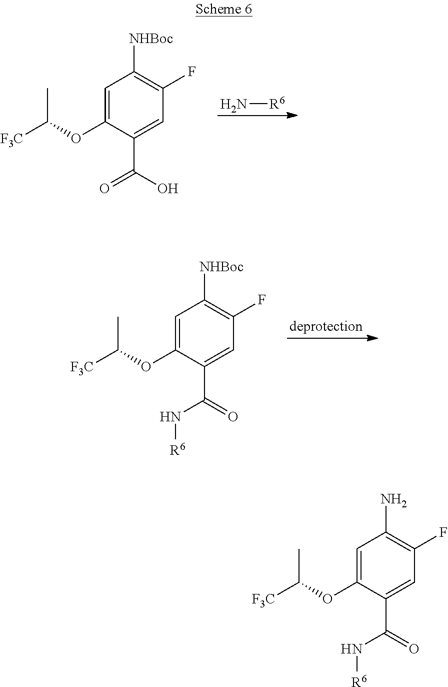

According to Scheme 6, (S)-4-((tert-butoxycarbonyl)amino)-5-fluoro-2-((1,1,1-trifluoropropan-2-yl)oxy)benzoic acid, as prepared in Example 40—Step B, is reacted with a commercially available or synthetically accessible amine R⁶—NH₂ using methods previously described. Subsequent deprotection using standard conditions affords the aniline intermediate.

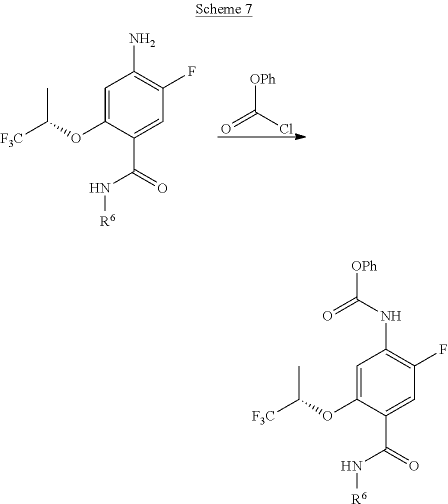

According to Scheme 7, aniline intermediates, prepared as shown in Scheme 5 and 6, are reacted with phenyl carbamate using pyridine, in an appropriates solvent, such as THF; in temperatures ranging from 40 to 70° C.

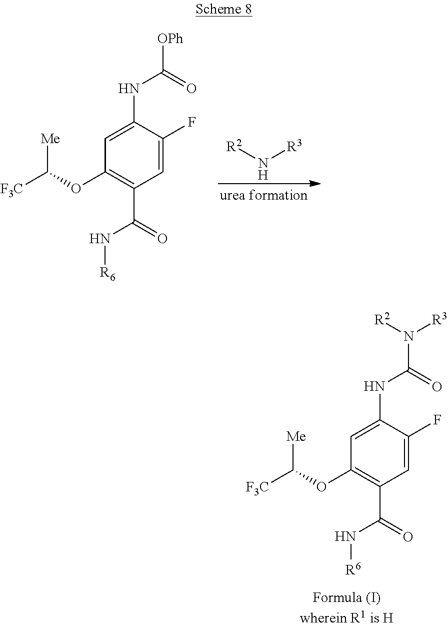

Compounds of Formula (I), wherein R², R³, and R⁶ are as defined above, may also be prepared s shown in Scheme 8 using methods known to one skilled in the art. The corresponding phenyl carbamate, prepared as shown in Scheme 7, is reacted with a synthetically accessible secondary amine, such as that prepared in Intermediate 3 and 5, in a polar aprotic solvent such as DCM, THE and the like, in the presence of a suitable base such as Et₃N, pyridine and the like, at elevated temperature of about 70° C.

Compounds of Formula (I), (II) or (III) may be converted to their corresponding salts using methods known to one of ordinary skill in the art. For example, an amine of Formula (III) is treated with trifluoroacetic acid, HCl, or citric acid in a solvent such as Et₂O, CH₂Cl₂, THF, MeOH, chloroform, or isopropanol to provide the corresponding salt form. Alternately, trifluoroacetic acid or formic acid salts are obtained as a result of reverse phase HPLC purification conditions. Crystalline forms of pharmaceutically acceptable salts of compounds of Formula (I) may be obtained in crystalline form by recrystallization from polar solvents (including mixtures of polar solvents and aqueous mixtures of polar solvents) or from non-polar solvents (including mixtures of non-polar solvents).

Where the compounds according to this invention have at least one chiral center, they may accordingly exist as enantiomers. Where the compounds possess two or more chiral centers, they may additionally exist as diastereomers. It is to be understood that all such isomers and mixtures thereof are encompassed within the scope of the present invention.

Compounds prepared according to the schemes described above may be obtained as single forms, such as single enantiomers, by form-specific synthesis, or by resolution. Compounds prepared according to the schemes above may alternately be obtained as mixtures of various forms, such as racemic (1:1) or non-racemic (not 1:1) mixtures. Where racemic and non-racemic mixtures of enantiomers are obtained, single enantiomers may be isolated using conventional separation methods known to one of ordinary skill in the art, such as chiral chromatography, recrystallization, diastereomeric salt formation, derivatization into diastereomeric adducts, biotransformation, or enzymatic transformation. Where regioisomeric or diastereomeric mixtures are obtained, as applicable, single isomers may be separated using conventional methods such as chromatography or crystallization.

The following specific examples are provided to further illustrate the invention and various preferred embodiments.

### Examples

In obtaining the compounds described in the examples below and the corresponding analytical data, the following experimental and analytical protocols were followed unless otherwise indicated.

Unless otherwise stated, reaction mixtures were magnetically stirred at room temperature (rt) under a nitrogen atmosphere. Where solutions were “dried,” they were generally dried over a drying agent such as Na₂SO₄ or MgSO₄. Where mixtures, solutions, and extracts were “concentrated”, they were typically concentrated on a rotary evaporator under reduced pressure.

Normal-phase silica gel chromatography (FCC) was performed on silica gel (SiO₂) using prepacked cartridges.

Preparative reverse-phase high performance liquid chromatography (RP HPLC) was performed on either:

METHOD A. A Gilson GX-281 semi-prep-HPLC with Phenomenex Synergi C18 (10 μm, 150×25 mm), or Boston Green ODS C18 (5 μm, 150×30 mm), and mobile phase of 5-99% ACN in water (with 0.225% FA) over 10 min and then hold at 100% ACN for 2 min, at a flow rate of 25 mL/min.
or
METHOD B. A Gilson GX-281 semi-prep-HPLC with Phenomenex Synergi C18 (10 μm, 150×25 mm), or Boston Green ODS C18 (5 μm, 150×30 mm), and mobile phase of 5-99% ACN in water (0.1% TFA) over 10 min and then hold at 100% ACN for 2 min, at a flow rate of 25 mL/min.
or
METHOD C. A Gilson GX-281 semi-prep-HPLC with Phenomenex Synergi C18 (10 μm, 150×25 mm), or Boston Green ODS C18 (5 μm, 150×30 mm), and mobile phase of 5-99% ACN in water (0.05% HCl) over 10 min and then hold at 100% ACN for 2 min, at a flow rate of 25 mL/min.
or
METHOD D. a Gilson GX-281 semi-prep-HPLC with Phenomenex Gemini C18 (10 μm, 150×25 mm), AD (10 μm, 250 mm×30 mm), or Waters XBridge C18 column (5 μm, 150×30 mm), mobile phase of 0-99% ACN in water (with 0.05% ammonia hydroxide v/v) over 10 min and then hold at 100% ACN for 2 min, at a flow rate of 25 mL/min.
or
METHOD E. a Gilson GX-281 semi-prep-HPLC with Phenomenex Gemini C18 (10 μm, 150×25 mm), or Waters XBridge C18 column (5 μm, 150×30 mm), mobile phase of 5-99% ACN in water (10 mM NH₄HCO₃) over 10 min and then hold at 100% ACN for 2 min, at a flow rate of 25 mL/min.
or
METHOD F. Teledyne ISCO ACCQPrep HIP150 semi-prep-HPLC with Phenomenex Gemini-NX C18 (5 μm, 150×30 mm), mobile phase of 10-100% ACN in water (10 mM NH₄OH) over 10 min and then hold at 100% ACN for 2 min, at a flow rate of 30 mL/min.

Preparative supercritical fluid high performance liquid chromatography (SFC) was performed either on a Thar 80 Prep-SFC system, or Waters 80Q Prep-SFC system from Waters. The ABPR was set to 100 bar to keep the CO₂ in SF conditions, and the flow rate may verify according to the compound characteristics, with a flow rate ranging from 50 g/min to 70 g/min. The column temperature was ambient temperature

Mass spectra (MS) were obtained on a SHIMADZU LCMS-2020 MSD or Agilent 1200\G6110A MSD using electrospray ionization (ESI) in positive mode unless otherwise indicated. Calculated (calcd.) mass corresponds to the exact mass.

Nuclear magnetic resonance (NMR) spectra were obtained on Bruker model AVIII 400 spectrometers. Definitions for multiplicity are as follows: s=singlet, d=doublet, t=triplet, q=quartet, m=multiplet, br=broad. It will be understood that for compounds comprising an exchangeable proton, said proton may or may not be visible on an NMR spectrum depending on the choice of solvent used for running the NMR spectrum and the concentration of the compound in the solution.

Chemical names were generated using ChemDraw Ultra 12.0, ChemDraw Ultra 14.0 (CambridgeSoft Corp., Cambridge, Mass.) or ACD/Name Version 10.01 (Advanced Chemistry).

Compounds designated as R* or S* are enantiopure compounds where the absolute configuration was not determined.

### Intermediate 1: (S)-Phenyl (4-((2-chloro-6-fluorophenyl)carbamoyl)-2-fluoro-5-((1,1,1-trifluoropropan-2-yl)oxy)phenyl)carbamate

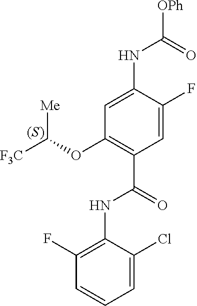

Step A. (S)-4-Bromo-5-fluoro-2-((1,1,1-trifluoropropan-2-yl)oxy)benzonitrile. To a solution of 4-bromo-2,5-difluorobenzonitrile (20 g, 91.7 mmol) in DMF (400 mL) was added K₂CO₃ (36.8 g, 266.1 mmol) and (S)-1,1,1-trifluoropropan-2-ol (11.5 g, 101 mmol) dropwise at 25° C. under N2. The mixture was stirred at 70° C. for 3 hr. The reaction mixture was diluted with H₂O (200 mL) and extracted with ethyl acetate (300 mL×3). The combined organic layer was washed with brine (300 mL), dried over Na₂SO₄, filtered and concentrated in vacuo. The residue was purified by column chromatography (petroleum ether/ethyl acetate=I/O to 10/1) to give the title compound as yellow solid. ¹H NMR (400 MHz, CDCl₃) δ=7.37 (d, J=7.2 Hz, 1H), 7.29 (d, J=5.4 Hz, 1H), 4.77-4.56 (m, 1H), 1.62 (dd, J=0.8, 6.4 Hz, 3H).

Step B. (S)-4-Bromo-5-fluoro-2-((1,1,1-trifluoropropan-2-yl)oxy)benzoic acid. A mixture of (S)-4-bromo-5-fluoro-2-((1,1,1-trifluoropropan-2-yl)oxy)benzonitrile (13 g, 41.7 mmol) in EtOH (52 mL) were added NaOH (6.23 g, 155.80 mmol) and H₂O (78 mL) was stirred at 90° C. for 16 hr under N2 atmosphere. The reaction mixture was quenched by HCl aqueous (1M, 200 mL) at 0° C., and then extracted with ethyl acetate (250 mL×2). The combined organic layers were washed with brine, dried over Na₂SO₄, filtered and concentrated in vacuo to give the title compound as brown solid. MS (ESI): mass calcd. for C₁₀H₇BrF₄O₃, 329.9; m/z found, 330.9 [M+H]⁺. ¹H NMR (400 MHz, CDCl₃) δ=7.89 (d, J=8.4 Hz, 1H), 7.30 (t, J=5.2 Hz, 1H), 4.82 (m, 1H), 1.63 (d, J=5.6 Hz, 3H).

Step C. (S)-4-Bromo-5-fluoro-2-((1,1,1-trifluoropropan-2-yl)oxy)benzoyl chloride. To a solution of (S)-4-bromo-5-fluoro-2-((1,1,1-trifluoropropan-2-yl)oxy)benzoic acid (13.3 g, 40.17 mmol) in DCM (100 mL) was added (COCl)₂ (5.10 g, 40.17 mmol) and DMF (29.36 mg, 401.74 μmol). The mixture was stirred at 25° C. for 1 hr. The mixture was concentrated in vacuo to give the title compound as brown solid.

Step D. (S)-4-Bromo-N-(2-chloro-6-fluorophenyl)-5-fluoro-2-((1,1,1-trifluoropropan-2-yl)oxy)benzamide. To a mixture of 2-chloro-6-fluoro-aniline (7.00 g, 48.07 mmol) and TEA (4.9 g, 48.1 mmol) was added (S)-4-bromo-5-fluoro-2-((1,1,1-trifluoropropan-2-yl)oxy)benzoyl chloride (14 g, 40.1 mmol) in DCM (100 mL). The mixture was stirred at 25° C. for 16.5 hr. The reaction mixture was quenched by addition of H₂O (200 mL) at 0° C., and then extracted with ethyl acetate (250 mL×2). The combined organic layers were washed with brine (200 mL), dried over Na₂SO₄, filtered and concentrated in vacuo. To the residue was added a solution of K₂CO₃ (5.54 g, 40.06 mmol) in MeOH (50 mL). The mixture was stirred 0.5 h at 25° C. The mixture filtered and concentrated in vacuo. The residue was purified by column chromatography (petroleum ether/ethyl acetate=1/1 to 10/1) to give the title compound as white solid. MS (ESI): mass calcd. for C₁₆H₁₀BrClF₅NO₂, 456.9; m/z found, 459.9 [M+H]⁺. ¹H NMR (400 MHz, CDCl₃) δ=8.92 (s, 1H), 8.09 (d, J=8.8 Hz, 1H), 7.29 (m, 2H), 7.12 (m, 2H), 4.91 (m, 1H), 1.67 (d, J=6.4 Hz, 3H).

Step E. (S)-4-Amino-N-(2-chloro-6-fluorophenyl)-5-fluoro-2-((1,1,1-trifluoropropan-2-yl)oxy)benzamide. A mixture of (S)-4-bromo-N-(2-chloro-6-fluorophenyl)-5-fluoro-2-((1,1,1-trifluoro propan-2-yl)oxy)benzamide (2 g, 4.36 mmol), diphenylmethanimine (1.58 g, 8.72 mmol), tris(dibenzylideneacetone)dipalladium (199.67 mg, 218.05 μmol), (5-diphenylphosphanyl-9,9-dimethyl-xanthen-4-yl)-diphenyl-phosphane (252.34 mg, 436.11 μmol) and Cs₂CO₃ (1.71 g, 5.23 mmol) in dioxane (30 mL) was degassed and purged with N2 for 3 times, and then the mixture was stirred at 95° C. for 16 hr under N2 atmosphere. The reaction mixture was diluted with H₂O (100 mL) and extracted with ethyl acetate (100 mL×3). The combined organic layers were washed with brine (200 mL), dried over Na₂SO₄, filtered and concentrated in vacuo. The residue was dissolved in THE (5 mL) and was added HCl (1M) (3 eq). The mixture was stirred at 25° C. for 3 hr. The reaction mixture was quenched with NaHCO₃ at 0° C. to adjust pH to 7. The mixture was diluted with H₂O (60 mL) and extracted with ethyl acetate (30 mL×3). The combined organic layers were washed with brine (60 mL), dried over Na₂SO₄, filtered and concentrated in vacuo. The residue was purified by column chromatography (petroleum ether/ethyl acetate=1/0 to 3/1) to give the title compound as yellow oil. MS (ESI): mass calcd. for C₁₆H₁₂ClF₅N₂O₂, 394.0; m/z found, 395.0 [M+H]⁺.

Step F. (S)-Phenyl (4-((2-chloro-6-fluorophenyl)carbamoyl)-2-fluoro-5-((1,1,1-trifluoropropan-2-yl)oxy)phenyl)carbamate. To a solution of (S)-4-amino-N-(2-chloro-6-fluorophenyl)-5-fluoro-2-((1,1,1-trifluoropropan-2-yl)oxy)benzamide (300 mg, 479 μmol) in DCM (2 mL) was added dropwise pyridine (114 mg, 1.44 mmol), and then a solution of phenyl carbonochloridate (97 mg, 622 μmol) in DCM (2 mL) was added dropwise in the mixture at 0° C. The resulting mixture was stirred at 25° C. for 12 hr. Then to the mixture was added phenyl carbonochloridate (97 mg, 622 μmol) and heated to 40° C. for 3 hr. The reaction mixture was diluted with H₂O (30 mL) and extracted with DCM (30 mL×3). The combined organic layers were washed with brine (60 mL), dried over Na₂SO₄, filtered and concentrated in vacuo. The residue was purified by column chromatography (petroleum ether/ethyl acetate=1/0 to 7/1) to give the title compound as yellow solid. MS (ESI): mass calcd. for C₂₃H₁₆ClF₅N₂O₄, 514.0; m/z found, 515.1 [M+H]⁺.

### Intermediate 2. Methyl (S)-5-fluoro-4-((phenoxycarbonyl)amino)-2-((1,1,1-trifluoropropan-2-yl)oxy)benzoate

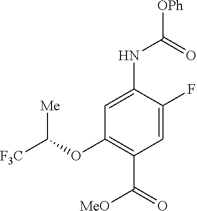 [SMILES: COC(=O)c1cc(F)c(NC(=O)Oc2ccccc2)cc1O[C@@H](C)C(F)(F)F]

To a solution of (S)-methyl 4-amino-5-fluoro-2-((1,1,1-trifluoropropan-2-yl)oxy)benzoate (Intermediate 7, 2.4 g, 8.4 mmol) in DCM (30 mL) was added dry pyridine (2.0 g, 25.3 mmol). A solution of phenyl carbonochloridate (1.58 g, 10.1 mmol) in DCM (10 mL) was added to the mixture at 0° C. The mixture was stirred at 25° C. for 12 hr. The reaction mixture was diluted with H₂O (20 mL) and extracted with ethyl acetate (30 mL×3). The combined organic layers were washed with aqueous HCl (1 M, 50 mL×2) and brine (50 mL×2), dried over anhydrous Na₂SO₄, filtered and concentrated in vacuo to give the title compound as a yellow oil. MS (ESI): mass calcd. for C₁₈H₁₅F₄NO₅, 401.1; m/z found, 402.3 [M+H]⁺. ¹H NMR (400 MHz, CDCl₃) δ=8.02 (d, J=6.4 Hz, 1H), 7.69 (d, J=7.2 Hz, 1H), 7.46-7.42 (m, 2H), 7.22-7.20 (m, 3H), 4.72-4.62 (m, 1H), 3.89 (s, 3H), 1.53 (dd, J=6.8 Hz, 3H).

### Intermediate 3. (R)-2-(((tert-Butyldiphenylsilyl)oxy)methyl)pyrrolidine

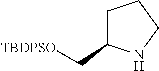

To a mixture of D-prolinol (3 g, 30 mmol) and imidazole (6 g, 89 mmol) in THE (30 mL) was added TBDPSCl (9.8 g, 36 mmol) dropwise at 0° C. under N2. The mixture was stirred at room temperature overnight. The mixture was diluted with ethyl acetate (30 mL) and washed with brine (60 mL), extracted with ethyl acetate (60 mL×3), dried with Na₂SO₄, filtered, concentrated under vacuum to give the residue. The residue was purified by flash column chromatography over silica gel (0-100% ethyl acetate/petroleum ether) to give the title compound as a yellow oil. MS (ESI): mass calcd. for C₂₁H₂₉NOSi, 339.20; m/z found, 340.2 [M+H]⁺. ¹H NMR (400 MHz, DMSO-d₆) δ=7.64-7.54 (m, 4H), 7.47-7.33 (m, 6H), 3.52-3.45 (m, 1H), 3.44-3.37 (m, 1H), 3.17-3.08 (m, 1H), 2.75-2.62 (m, 2H), 1.75-1.64 (m, 1H), 1.55 (quin, J=6.7 Hz, 2H), 1.43-1.31 (m, 1H), 0.96 (s, 9H).

### Intermediate 4. (S)-Phenyl (4-((3-chloro-2-methoxy-5-methylpyridin-4-yl) carbamoyl)-2-fluoro-5-((1,1,1-trifluoropropan-2-yl)oxy)phenyl)carbamate

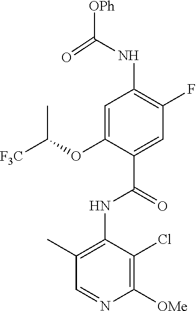 [SMILES: COc1ncc(C)c(NC(=O)c2cc(F)c(NC(=O)Oc3ccccc3)cc2O[C@@H](C)C(F)(F)F)c1Cl]

Step A. (S)-4-Bromo-N-(3-chloro-2-methoxy-5-methylpyridin-4-yl)-5-fluoro-2-((1,1,1-trifluoropropan-2-yl)oxy)benzamide. POCl₃ (4.9 g, 32.1 mmol) was added to a mixture of (S)-4-bromo-5-fluoro-2-((1,1,1-trifluoropropan-2-yl)oxy)benzoic acid (1.5 g, 4.0 mmol), 3-chloro-2-methoxy-5-methylpyridin-4-amine (690 mg, 4.0 mmol), pyridine (6.3 g, 80.2 mmol) in DCM (50 mL) at room temperature. The reaction mixture was stirred at room temperature for 2 hours. The reaction mixture was quenched with sat. aq. K₂CO₃ (30 mL) and extracted with DCM (50 mL×3). The combined organic layers were dried over Na₂SO₄, filtered and concentrated in vacuum. The residue was purified by column chromatography (SiO₂, gradient eluent: petroleum ether/ethyl acetate=100/0 to 60/40) to give the title compound as a yellow oil. MS (ESI): mass calcd. for C₁₇H₁₄BrClF₄N₂O₃, 484.0; m/z found, 486.8 [M+H]⁺. ¹H NMR (500 MHz, CDCl₃) δ=9.09 (s, 1H), 8.06 (d, J=9.0 Hz, 1H), 7.96 (s, 1H), 4.98-4.85 (m, 1H), 4.04-3.97 (m, 3H), 2.27-2.13 (m, 3H), 1.66 (d, J=6.4 Hz, 3H); ¹⁹F NMR (471 MHz, CDCl₃) δ=−72.73-−82.03 (m, 1F), −113.22 (s, 1F).

Step B. (S)-N-(3-Chloro-2-methoxy-5-methylpyridin-4-yl)-4-((diphenylmethylene)amino)-5-fluoro-2-((1,1,1-trifluoropropan-2-yl)oxy)benzamide. A mixture of (S)-4-bromo-N-(3-chloro-2-methoxy-5-methylpyridin-4-yl)-5-fluoro-2-((1,1,1-trifluoropropan-2-yl)oxy)benzamide (700 mg, 1.4 mmol), diphenylmethanimine (277 mg, 1.5 mmol), and Cs₂CO₃ (1.36 g, 4.2 mmol) in toluene (10 mL) was purged with N₂. Then Xantphos (161 mg, 278.24 μmol) and Pd₂(dba)₃ (127 mg, 139 μmol) were added into the mixture reaction. The reaction mixture was purged with N2 and heated at 100° C. overnight. The mixture was diluted with water (20 mL) and extracted with ethyl acetate (20 mL×3). The organic layer was dried over Na₂SO₄, filtered and evaporated under vacuum. The residue was purified by column chromatography (SiO₂, gradient elution: petroleum ether/ethyl acetate=100/0 to 70/30) to give the title compound as a yellow oil. MS (ESI): mass calcd. for C₃₀H₂₄ClF₄N₃O₃, 585.1; m/z found, 586.2 [M+H]⁺. ¹H NMR (400 MHz, CDCl₃) δ=9.10 (s, 1H), 7.98-7.89 (m, 1H), 7.84 (d, J=10.8 Hz, 1H), 7.76 (br d, J=7.5 Hz, 2H), 7.55-7.48 (m, 1H), 7.46-7.39 (m, 2H), 7.34 (br d, J=7.3 Hz, 1H), 7.17 (br d, J=6.4 Hz, 2H), 6.38 (d, J=6.2 Hz, 1H), 4.62 (td, J=6.2, 12.3 Hz, 1H), 3.99 (s, 3H), 2.15 (s, 3H), 1.62 (s, 2H), 1.42 (d, J=6.4 Hz, 3H); ¹⁹F NM R (376 MHz, CDCl₃) δ=−78.22 (d, J=5.9 Hz, 1F), −126.94-−134.76 (m, 1F).

Step C. (S)-4-Amino-N-(3-chloro-2-methoxy-5-methylpyridin-4-yl)-5-fluoro-2-((1,1,1-trifluoropropan-2-yl)oxy)benzamide. A mixture of (S)-N-(3-chloro-2-methoxy-5-methylpyridin-4-yl)-4-((diphenylmethylene)amino)-5-fluoro-2-((1,1,1-trifluoropropan-2-yl)oxy)benzamide (800 mg, 1.28 mmol) and 4 M HCl solution in dioxane (0.6 mL, 2.4 mmol) in MeOH (10 mL) was stirred at room temperature for 2 hours. The reaction mixture was concentrated and the pH was adjusted to 8 by sat. aq. NaHCO₃. The mixture was extracted by ethyl acetate (20 mL). The organic layer was washed by brine (15 mL), dried by Na₂SO₄, filtered and concentrated to give the title compound as a yellow oil. MS (ESI): mass calcd. for C₁₇H₁₆ClF₄N₃O₃, 421.1; m/z found, 422.1 [M+H]⁺.

Step D. (S)-Phenyl (4-((3-chloro-2-methoxy-5-methylpyridin-4-yl)carbamoyl)-2-fluoro-5-((1,1,1-trifluoropropan-2-yl)oxy)phenyl)carbamate. To a solution consisting of (S)-4-amino-N-(3-chloro-2-methoxy-5-methylpyridin-4-yl)-5-fluoro-2-((1,1,1-trifluoropropan-2-yl)oxy)benzamide (750 mg, crude) and phenyl carbonochloridate (362 mg, 2.3 mmol) in THE (5 mL) was added pyridine (183 mg, 2.3 mmol) at 0° C. The mixture was stirred at 40° C. for 2 hours. The mixture was concentrated to give the title compound as a yellow oil. MS (ESI): mass calcd. for C₂₄H₂₀ClF₄N₃O₅, 5 41.1; m/z found, 542.1 [M+H]⁺. ¹H NMR (400 MHz, CDCl₃) δ=9.22 (s, 1H), 8.20-8.07 (m, 2H), 7.98 (s, 1H), 7.58-7.43 (m, 3H), 7.36-7.31 (m, 1H), 7.24 (d, J=7.9 Hz, 2H), 4.98 (spt, J=6.2 Hz, 1H), 4.04 (s, 3H), 2.23 (s, 3H), 1.64 (d, J=6.4 Hz, 3H); ¹⁹F NMR (376 MHz, CDCl₃) δ=−73.40-−80.74 (m, 1F), −138.75 (br s, 1F).

### Intermediate 5. N-Ethyl-2,2-difluoroethan-1-amine hydrochloride salt

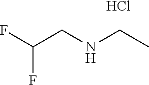

Step A. tert-Butyl (2,2-difluoroethyl)carbamate. To a mixture of 2,2-difluoroethylamine (3 g, 37 mmol) and Et₃N (7.5 g, 74 mmol) in DCM (30 mL) was added Boc₂O (8.9 g, 41 mmol), and the mixture was stirred at room temperature overnight. The mixture was diluted with DCM and washed with saturated aqueous citric acid solution. The organic phase was separated, dried over Na₂SO₄, filtered and concentrated under vacuum. It was purified by silica column chromatography (gradient elution: 0-10% EtOAc in petroleum ether) to give the title compound as a colorless solid. ¹H NMR (400 MHz, CHLOROFORM-d) δ ppm 5.61-6.03 (m, 1H), 4.80 (br s, 1H), 3.36-3.60 (m, 2H), 1.46 (s, 9H); ¹⁹F NMR (376 MHz, CHLOROFORM-d) δ ppm −123.53 (dt, J=55.9, 14.6 Hz, 1F).

Step B. tert-Butyl (2,2-difluoroethyl)(ethyl)carbamate. NaH (60% dispersion in mineral oil, 485 mg, 12 mmol) was slowly added into the solution of tert-butyl (2,2-difluoroethyl)carbamate (2.0 g, 11 mmol) in DMF (15 mL) in ice-water bath. The reaction mixture was stirred at room temperature for half an hour. Then EtBr (1.6 g, 14.4 mmol) was added and the reaction mixture was further stirred at room temperature for 2 hours. The reaction was quenched with water and extracted with EtOAc. The organic layer was washed with water and brine, dried over Na₂SO₄, filtered and concentrated. The crude product was purified by flash column chromatography over silica gel (gradient elution: 0-30% EtOAc in petroleum ether) to give the title compound as a light yellow oil. ¹H NMR (400 MHz, CHLOROFORM-d) δ ppm 5.59-6.21 (m, 1H), 3.38-3.58 (m, 2H), 3.28 (s, 2H), 1.44 (d, J=0.9 Hz, 9H), 1.09 (t, J=6.8 Hz, 3H).

Step C. N-Ethyl-2,2-difluoroethan-1-amine hydrochloride salt. A mixture of tert-butyl (2,2-difluoroethyl)(ethyl)carbamate (1.6 g, 7.7 mmol) and HCl (4 M MeOH solution, 3 mL, 12 mmol) in DCM (10 mL) was stirred at room temperature for 3 hours. The reaction was concentrated to give the title compound as white solid. ¹H NMR (400 MHz, DMSO-d₆) δ ppm 6.17-6.66 (m, 1H), 3.47 (t, J=16.0 Hz, 2H), 2.99 (q, J=6.9 Hz, 2H), 1.17 (t, J=7.2 Hz, 3H).

### Intermediate 6. Methyl (S)-4-((diphenylmethylene)amino)-5-fluoro-2-((1,1,1-trifluoropropan-2-yl)oxy)benzoate

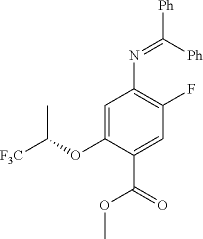 [SMILES: COC(=O)c1cc(F)c(N=C(c2ccccc2)c2ccccc2)cc1O[C@@H](C)C(F)(F)F]

Step A. (S)-Methyl 4-bromo-5-fluoro-2-((1,1,1-trifluoropropan-2-yl)oxy)benzoate. A mixture of (S)-4-bromo-5-fluoro-2-((1,1,1-trifluoropropan-2-yl)oxy)benzoic acid (Intermediate 1, Step B, 4 g, 12.1 mmol) in MeOH (70 mL) was cooled to −10° C. under nitrogen then dropwise addition of SOCl₂ (2.87 g, 24.2 mmol) was added while maintaining the temperature between −10° C. to −5° C. The mixture was stirred at 70° C. for 4 hours. The reaction mixture was concentrated in vacuo to remove MeOH. The residue was diluted with H₂O (60 mL) and extracted with DCM (60 mL×3). The combined organic layers were washed with brine (60 mL×2), dried over Na₂SO₄, filtered and concentrated in vacuo. The residue was purified by column chromatography (petroleum ether/ethyl acetate=1/0) to give the title compound as a colorless oil. MS (ESI): mass calcd. for C₁₁H₉BrF₄O₃, 344.0; m/z found, 346.8 [M+H]⁺. ¹H NMR (400 MHz, CDCl₃) δ=7.64 (d, J=4.8 Hz, 1H), 7.26 (m, 1H), 4.65 (m, 1H), 3.92 (s, 3H), 1.61 (m, 3H).

Step B. Methyl (S)-4-((diphenylmethylene)amino)-5-fluoro-2-((1,1,1-trifluoropropan-2-yl)oxy)benzoate. A mixture of methyl (S)-4-bromo-5-fluoro-2-((1,1,1-trifluoropropan-2-yl)oxy)benzoate (3.0 g, 8.7 mmol), diphenylmethanimine (2.1 g, 11.3 mmol), and Cs₂CO₃ (4.3 g, 13.0 mmol) in toluene (15 mL) were purged with N2. Then xantphos (1.0 g, 1.7 mmol) and Pd₂(dba)₃ (0.8 g, 0.87 mmol) were added into the mixture reaction. The reaction mixture was purged with N2 and heated at 100° C. overnight then cooled down to room temperature. The mixture was diluted by water and extracted with EtOAc. The organic layer was dried over Na₂SO₄, filtered and concentrated. The crude product was purified by flash column chromatography over silica gel (gradient elution: 0-30% EtOAc in petroleum ether) to give the title compound as a yellow oil. ESI-MS: m/z 446.1 [M+H]⁺. ¹H NMR (400 MHz, CHLOROFORM-d) δ ppm 7.80-7.84 (m, 1H), 7.57-7.66 (m, 1H), 7.39-7.56 (m, 6H), 7.27-7.37 (m, 3H), 6.42 (d, J=6.5 Hz, 1H), 4.39 (dt, J=12.5, 6.3 Hz, 1H), 3.84 (s, 3H), 1.37 (d, J=6.5 Hz, 3H); ¹⁹F NMR (376 MHz, CHLOROFORM-d) δ ppm −78.18 (s, 1F), −130.98 (br s, 1F).

### Intermediate 7. (S)-Methyl 4-amino-5-fluoro-2-((1,1,1-trifluoropropan-2-yl)oxy)benzoate

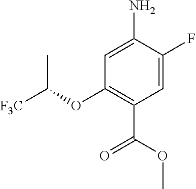 [SMILES: COC(=O)c1cc(F)c(N)cc1O[C@@H](C)C(F)(F)F]

A mixture of methyl (S)-4-((diphenylmethylene)amino)-5-fluoro-2-((1,1,1-trifluoropropan-2-yl)oxy)benzoate (Intermediate 6, 1.2 g, 1.8 mmol) and HCl (4 M dioxane solution, 0.6 mL, 2.4 mmol) in MeOH (5 mL) was stirred at room temperature for 3 hours. The reaction mixture was concentrated and the pH was adjusted to 8 by saturated aqueous NaHCO₃ solution. The mixture was extracted by EtOAc. The organic layer was washed by brine, dried by Na₂SO₄, filtered and concentrated. The residue was purified by flash column chromatography over silica gel (gradient elution: 0-100% EtOAc in petroleum ether) to give the title compound as a yellow oil. ¹H NMR (400 MHz, CHLOROFORM-d) δ ppm 7.56 (d, J=11.7 Hz, 1H), 6.37 (d, J=7.6 Hz, 1H), 4.53 (dt, J=12.7, 6.3 Hz, 1H), 4.12 (s, 2H), 3.82 (s, 3H), 1.50 (d, J=6.6 Hz, 3H); ¹⁹F NMR (376 MHz, CHLOROFORM-d) δ ppm 10.88-11.93 (m, 1F), −77.89 (s, 1F), −141.71 (s, 1F); ESI-MS: m/z 282.1 [M+H]⁺.

### Intermediate 8. (S)-4-(3-(2,2-Difluoroethyl)-3-ethylureido)-5-fluoro-2-((1,1,1-trifluoropropan-2-yl)oxy)benzoic acid

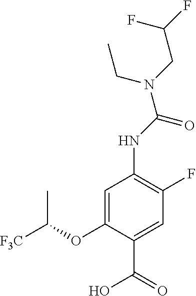 [SMILES: CCN(CC(F)F)C(=O)Nc1cc(O[C@@H](C)C(F)(F)F)c(C(=O)O)cc1F]

Step A. Methyl (S)-4-(3-(2,2-difluoroethyl)-3-ethylureido)-5-fluoro-2-((1,1,1-trifluoropropan-2-yl)oxy)benzoate. To a solution of methyl (S)-5-fluoro-4-((phenoxycarbonyl)amino)-2-((1,1,1-trifluoropropan-2-yl)oxy)benzoate (Intermediate 2, 500 mg, crude) in THE (5 mL) was added N-ethyl-2,2-difluoroethan-1-amine hydrochloride salt (Intermediate 5, 236 mg, 1.6 mmol, HCl salt) and pyridine (148 mg, 1.9 mmol). The mixture reaction was stirred at 80° C. for 16 hours then cooled down to room temperature. The mixture was quenched with saturated aqueous NH₄Cl solution and extracted with EtOAc The combined organic layers were dried over Na₂SO₄, filtered and concentrated. The residue was purified by silica gel column chromatography over silica gel (gradient elution: 0-60% EtOAc in petroleum ether) to give the title compound as yellow oil. ESI-MS: m/z 417.1 [M+H]⁺

Step B. (S)-4-(3-(2,2-Difluoroethyl)-3-ethylureido)-5-fluoro-2-((1,1,1-trifluoropropan-2-yl)oxy)benzoic acid. LiOH.H₂O (40 mg, 1.7 mmol) was slowly added into the solution of methyl (S)-4-(3-(2,2-difluoroethyl)-3-ethylureido)-5-fluoro-2-((1,1,1-trifluoropropan-2-yl)oxy)benzoate (270 mg, 0.6 mmol) in THF/H₂O (v/v, 3/1, 4 mL) at room temperature. The reaction was heated to 40° C. for 16 h. The mixture was diluted by EtOAc and water, adjusted the pH to 6 by 1 M HCl aqueous solution. The organic layer was washed by brine, dried by Na₂SO₄, filtered and concentrated. The residue was purified by flash column chromatography over silica gel (gradient elution: 0-60% EtOAc in petroleum ether) to give the title compound as a yellow solid. ¹H NMR (400 MHz, CHLOROFORM-d) δ ppm 8.21 (d, J=6.0 Hz, 1H), 7.91 (d, J=11.5 Hz, 1H), 7.08 (s, 1H), 5.85-6.21 (m, 1H), 4.96 (dt, J=12.2, 6.1 Hz, 1H), 3.71 (td, J=14.1, 4.1 Hz, 2H), 3.51 (q, J=7.3 Hz, 2H), 1.63 (d, J=6.5 Hz, 3H), 1.34 (t, J=7.3 Hz, 3H); ¹⁹F NMR (376 MHz, CHLOROFORM-d) δ ppm −78.51 (s, 1F), −121.28 (br s, 1F), −138.67 (s, 1F); ESI-MS: m/z 403.1 [M+H]⁺

### Example 1: (S)-N-(2-Chloro-6-fluorophenyl)-4-(3,3-diethylureido)-5-fluoro-2-((1,1,1-trifluoropropan-2-yl)oxy)benzamide

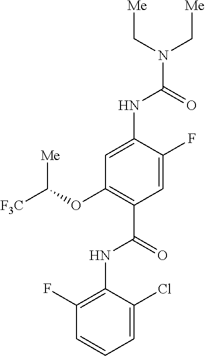 [SMILES: CCN(CC)C(=O)Nc1cc(O[C@@H](C)C(F)(F)F)c(C(=O)Nc2c(F)cccc2Cl)cc1F]

To a mixture of N-ethylethanamine (6.82 mg, 93.23 μmol), TEA (15.72 mg, 155.39 μmol) in DCM (1 mL) was added phenyl N-[4-[(2-chloro-6-fluoro-phenyl) carbamoyl]-2-fluoro-5-[(1S)-2,2,2-trifluoro-1-methyl-ethoxy]phenyl]carbamate (40 mg, 77.70 μmol). The mixture was stirred at 25° C. for 1 hr. The mixture was filtered and concentrated under reduced pressure. The residue was purified by prep-HPLC (method A) to give the title compound as white solid. MS (ESI): mass calcd. for C₂₁H₂₁ClF₅N₃O₃, 493.1; m/z found, 494.1 [M+H]⁺. ¹H NMR (400 MHz, DMSO-d₆) δ=9.00-8.72 (m, 1H), 7.88-7.66 (m, 2H), 7.48 (m, 1H), 5.52-5.32 (m, 1H), 4.78 (m, 1H), 4.65-4.41 (m, 3H), 4.26 (m, 1H), 3.93 (m, 1H), 3.58 (m, 1H), 3.09-2.90 (m, 2H), 2.86-2.65 (m, 2H).

### Example 2: (S)-N-(4-((2-Chloro-6-fluorophenyl)carbamoyl)-2-fluoro-5-((1,1,1-trifluoropropan-2-yl)oxy)phenyl)piperidine-1-carboxamide

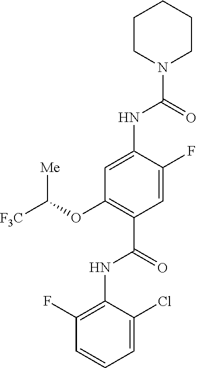 [SMILES: C[C@H](Oc1cc(NC(=O)N2CCCCC2)c(F)cc1C(=O)Nc1c(F)cccc1Cl)C(F)(F)F]

The title compound was prepared in a manner analogous to Example 1, however substituting piperidine for N-ethylethanamine. MS (ESI): mass calcd. for C₂₂H₂₁ClF₅N₃O₃, 505.1; m/z found, 506.1 [M+H]⁺. ¹H NMR (400 MHz, CDCl₃) δ=8.37-8.27 (m, 1H), 7.60 (s, 1H), 7.53 (s, 1H), 7.33 (m, 1H), 4.79-4.70 (m, 2H), 4.50 (m, 1H), 3.93 (m, 2H), 3.78-3.57 (m, 1H), 3.41 (s, 3H), 3.22-3.16 (m, 1H), 3.12-2.65 (m, 4H).

### Example 3: (S)-N-(2-Chloro-6-fluorophenyl)-4-(3-ethyl-3-(2-hydroxyethyl)ureido)-5-fluoro-2-((1,1,1-trifluoropropan-2-yl)oxy)benzamide

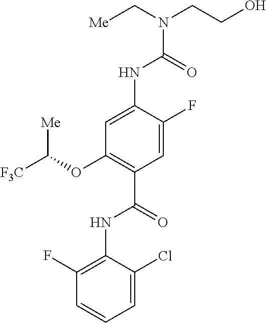 [SMILES: CCN(CCO)C(=O)Nc1cc(O[C@@H](C)C(F)(F)F)c(C(=O)Nc2c(F)cccc2Cl)cc1F]

The title compound was prepared in a manner analogous to Example 1, however substituting 2-(ethylamino)ethanol for N-ethylethanamine. MS (ESI): mass calcd. for C₂₁H₂₁ClF₅N₃O₄, 509.1; m/z found, 510.1 [M+H]⁺. ¹H NMR (400 MHz, CDCl₃) δ=9.10 (s, 1H), 8.77-8.54 (m, 1H), 8.15 (d, J=6.4 Hz, 1H), 7.99 (d, J=12.0 Hz, 1H), 7.30-7.27 (m, 1H), 7.21 (d, J=5.6 Hz, 1H), 7.11 (s, 1H), 5.07-4.95 (m, 1H), 3.97-3.90 (m, 2H), 3.62-3.51 (m, 2H), 3.50-3.42 (m, 2H), 2.59 (s, 1H), 1.66-1.60 (m, 3H), 1.27 (s, 3H).

### Example 4: (S)-N-(2-Chloro-6-fluorophenyl)-4-(3-ethyl-3-isopropylureido)-5-fluoro-2-((1,1,1-trifluoropropan-2-yl)oxy)benzamide

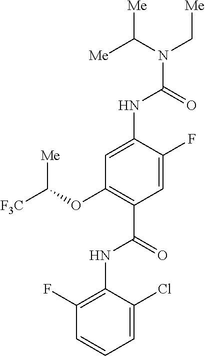 [SMILES: CCN(C(=O)Nc1cc(O[C@@H](C)C(F)(F)F)c(C(=O)Nc2c(F)cccc2Cl)cc1F)C(C)C]

The title compound was prepared in a manner analogous to Example 1, however substituting N-ethylpropan-2-amine for N-ethylethanamine. MS (ESI): mass calcd. For C₂₂H₂₃ClFsN₃O₃, 507.1; m/z found, 508.1 [M+H]⁺. ¹H NMR (400 MHz, CDCl₃) δ=9.10 (s, 1H), 8.30 (d, J=6.4 Hz, 1H), 8.04 (d, J=12.4 Hz, 1H), 7.29 (s, 1H), 7.22 (dt, J=5.6, 8.0 Hz, 1H), 7.15-7.08 (m, 1H), 6.91 (d, J=4.4 Hz, 1H), 5.04 (td, J=6.0, 12.4 Hz, 1H), 4.53 (td, J=6.4, 13.6 Hz, 1H), 3.33 (q, J=7.2 Hz, 2H), 1.64 (d, J=6.4 Hz, 3H), 1.34 (t, J=7.2 Hz, 3H), 1.26 (d, J=6.8 Hz, 6H).

### Example 5: (S)-N-(2-Chloro-6-fluorophenyl)-4-(3-cyclopropyl-3-ethylureido)-5-fluoro-2-((1,1,1-trifluoropropan-2-yl)oxy)benzamide

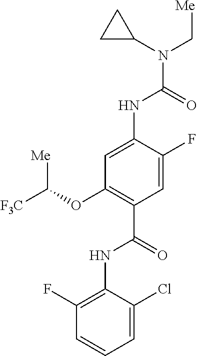 [SMILES: CCN(C(=O)Nc1cc(O[C@@H](C)C(F)(F)F)c(C(=O)Nc2c(F)cccc2Cl)cc1F)C1CC1]

The title compound was prepared in a manner analogous to Example 1, however substituting N-ethylcyclopropanamine for N-ethylethanamine. MS (ESI): mass calcd. For C₂₂H₂₁ClF₅N₃O₃, 505.1; m/z found, 506.1 [M+H]⁺. ¹H NMR (400 MHz, CDCl₃) δ=9.11 (s, 1H), 8.34 (d, J=6.4 Hz, 1H), 8.05 (d, J=12.0 Hz, 1H), 7.99 (d, J=4.0 Hz, 1H), 7.29 (s, 1H), 7.22 (dt, J=5.6, 8.0 Hz, 1H), 7.15-7.09 (m, 1H), 5.04 (td, J=6.4, 12.4 Hz, 1H), 3.59-3.38 (m, 2H), 2.77-2.64 (m, 1H), 1.64 (d, J=6.4 Hz, 3H), 1.23 (t, J=7.2 Hz, 3H), 1.12-1.02 (m, 2H), 0.97-0.86 (m, 2H).

### Example 6: (S)-N-(2-Chloro-6-fluorophenyl)-4-(3-ethyl-3-(2-methoxyethyl)ureido)-5-fluoro-2-((1,1,1-trifluoropropan-2-yl)oxy)benzamide

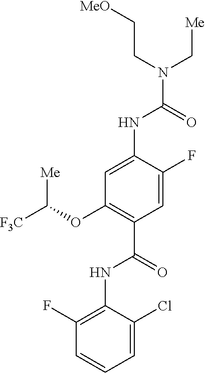 [SMILES: CCN(CCOC)C(=O)Nc1cc(O[C@@H](C)C(F)(F)F)c(C(=O)Nc2c(F)cccc2Cl)cc1F]

The title compound was prepared in a manner analogous to Example 1, however substituting N-ethyl-2-methoxyethanamine for N-ethylethanamine. MS (ESI): mass calcd. For C₂₂H₂₃ClF₅N₃O₄, 523.1; m/z found, 524.0 [M+H]⁺. ¹H NMR (400 MHz, CDCl₃) δ=9.16-8.95 (m, 2H), 8.10 (d, J=6.4 Hz, 1H), 8.00 (d, J=12.4 Hz, 1H), 7.27-7.17 (m, 2H), 7.14-7.08 (m, 1H), 5.12-4.92 (m, 1H), 3.61 (s, 2H), 3.51 (s, 2H), 3.51-3.49 (m, 3H), 3.48-3.41 (m, 2H), 1.63 (d, J=6.4 Hz, 3H), 1.27-1.22 (m, 3H)

### Example 7: (S)-N-(2-chloro-6-fluorophenyl)-4-(3-(2,2-difluoroethyl)-3-ethylureido)-5-fluoro-2-((1,1,1-trifluoropropan-2-yl)oxy)benzamide

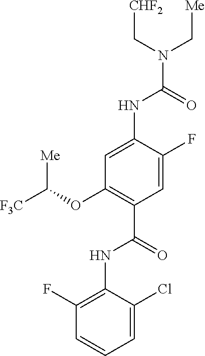 [SMILES: CCN(CC(F)F)C(=O)Nc1cc(O[C@@H](C)C(F)(F)F)c(C(=O)Nc2c(F)cccc2Cl)cc1F]

The title compound was prepared in a manner analogous to Example 1, however substituting N-ethyl-2,2-difluoroethanamine for N-ethylethanamine. MS (ESI): mass calcd. For C₂₁H₁₉ClF₇N₃O₃, 529.1; m/z found, 530.2 [M+H]⁺. ¹H NMR (400 MHz, CDCl₃) δ=9.08 (s, 1H), 8.19 (d, J=6.4 Hz, 1H), 8.07 (d, J=12.0 Hz, 1H), 7.31-7.27 (m, 1H), 7.26-7.19 (m, 1H), 7.16-7.09 (m, 1H), 7.07-7.02 (m, 1H), 6.43-5.71 (m, 1H), 5.09-4.91 (m, 1H), 3.79-3.67 (m, 2H), 3.57-3.48 (m, 2H), 1.65 (d, J=6.4 Hz, 3H), 1.35 (t, J=7.2 Hz, 3H).

### Example 8: (S)-N-(2-Chloro-6-fluorophenyl)-4-(3-ethylureido)-5-fluoro-2-((1,1,1-trifluoropropan-2-yl)oxy)benzamide

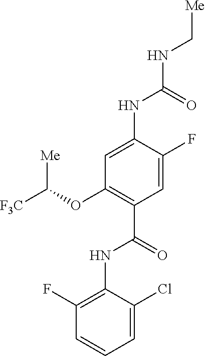 [SMILES: CCNC(=O)Nc1cc(O[C@@H](C)C(F)(F)F)c(C(=O)Nc2c(F)cccc2Cl)cc1F]

To a solution of (S)-4-amino-N-(2-chloro-6-fluorophenyl)-5-fluoro-2-((1,1,1-trifluoropropan-2-yl)oxy)benzamide (Intermediate 1, Step E, 50 mg, 127 μmol) in DCM (2 mL) was added dropwise TEA (38 mg, 380 μmol) at 0° C. Then isocyanatoethane (10.8 mg, 152 μmol) in DCM (1 mL) was added dropwise at 0° C. The resulting mixture was stirred at 40° C. for 12 hr. Then the mixture was concentrated in vacuo. Then a solution of the residue in DCE (3 mL) was stirred at 80° C. for 12 hr. The reaction mixture was concentrated in vacuo. The residue was purified by reversed-phase HPLC (method A) to give the title compound as white solid. MS (ESI): mass calcd. for C₁₉H₁₇ClF₅N₃O₃, 465.1; m/z found, 466.3 [M+H]⁺. ¹H NMR (400 MHz, CDCl₃) δ=9.15 (s, 1H), 8.30 (d, J=6.4 Hz, 1H), 8.03 (d, J=12.4 Hz, 1H), 7.31-7.28 (m, 1H), 7.25-7.20 (m, 1H), 7.15-7.09 (m, 1H), 7.07-7.02 (m, 1H), 5.21-5.10 (m, 1H), 5.07-4.97 (m, 1H), 3.29-3.21 (m, 2H), 1.64 (d, J=6.4 Hz, 3H), 1.16 (t, J=7.2 Hz, 3H).

### Example 9: (S)-N-(2-Chloro-6-fluorophenyl)-5-fluoro-4-(3-isopropylureido)-2-((1,1,1-trifluoropropan-2-yl)oxy)benzamide

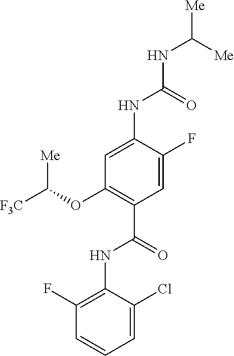 [SMILES: CC(C)NC(=O)Nc1cc(O[C@@H](C)C(F)(F)F)c(C(=O)Nc2c(F)cccc2Cl)cc1F]

To a solution of (S)-4-amino-N-(2-chloro-6-fluorophenyl)-5-fluoro-2-((1,1,1-trifluoropropan-2-yl)oxy)benzamide (Intermediate 1, Step E, 50 mg, 127 μmol) in DCM (1 mL) was added dropwise TEA (38.45 mg, 380.01 μmol, 52.89 μL). The mixture was stirred at 0° C. Then a solution of 2-isocyanatopropane (13 mg, 152 μmol) in DCM (2 mL) was added dropwise at 0° C. The resulting mixture was stirred at 40° C. for 16 hr. Then the mixture was concentrated in vacuo. A solution of the residue in DCE (3 mL) was stirred at 80° C. for 12 h. The mixture was further stirred at 100° C. for 24 hr. The reaction mixture was concentrated in vacuo. The residue was purified by prep-HPLC (method A) to give the title compound as white solid. MS (ESI): mass calcd. for C₂₀H₁₉ClF₅N₃O₃, 479.1; m/z found, 480.2 [M+H]⁺. ¹H NMR (400 MHz, CDCl₃) δ=9.08 (s, 1H), 8.26 (d, J=6.4 Hz, 1H), 8.02 (d, J=12.0 Hz, 1H), 7.32-7.28 (m, 1H), 7.25-7.18 (m, 1H), 7.15-7.07 (m, 1H), 6.67 (br d, J=3.2 Hz, 1H), 5.08-4.94 (m, 1H), 4.60 (s, 1H), 4.09-3.97 (m, 1H), 1.64 (d, J=6.8 Hz, 3H), 1.24 (s, 6H).

### Example 10: (S)-N-(2-Chloro-6-fluorophenyl)-4-(3,3-dimethylureido)-5-fluoro-2-((1,1,1-trifluoropropan-2-yl)oxy)benzamide

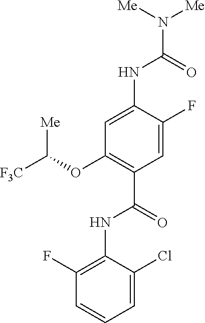 [SMILES: C[C@H](Oc1cc(NC(=O)N(C)C)c(F)cc1C(=O)Nc1c(F)cccc1Cl)C(F)(F)F]

The title compound was prepared in a manner analogous to Example 1, however substituting dimethylamine for N-ethylethanamine. MS (ESI): mass calcd. For C₁₉H₁₇ClF₅N₃O₃, 465.09; m/z found, 466.0 [M+H]⁺. ¹H NMR (400 MHz, CDCl₃) δ=9.14-9.06 (m, 1H), 8.32-8.27 (m, 1H), 8.10-8.02 (m, 1H), 7.28-7.20 (m, 2H), 7.17-7.09 (m, 1H), 6.94-6.86 (m, 1H), 5.09-4.98 (m, 1H), 3.14-3.11 (m, 6H), 1.67-1.63 (m, 3H).

### Example 11: (S)-N-(2-Chloro-6-fluorophenyl)-4-(3-ethyl-3-methylureido)-5-fluoro-2-((1,1,1-trifluoropropan-2-yl)oxy)benzamide

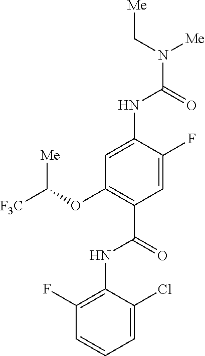 [SMILES: CCN(C)C(=O)Nc1cc(O[C@@H](C)C(F)(F)F)c(C(=O)Nc2c(F)cccc2Cl)cc1F]

The title compound was prepared in a manner analogous to Example 1, however substituting N-methylethanamine for N-ethylethanamine. MS (ESI): mass calcd. For C₂₀H₁₉ClF₅N₃O₃, 479.1; m/z found, 480.1 [M+H]⁺. ¹H NMR (400 MHz, CDCl₃) δ=9.09 (s, 1H), 8.29 (d, J=6.4 Hz, 1H), 8.04 (d, J=12.4 Hz, 1H), 7.27-7.18 (m, 2H), 7.16-7.07 (m, 1H), 6.92-6.83 (m, 1H), 5.09-4.96 (m, 1H), 3.53-3.42 (m, 2H), 3.08 (s, 3H), 1.64 (d, J=6.4 Hz, 3H), 1.26 (t, J=7.2 Hz, 3H).

### Example 12: N-(4-((2-Chloro-6-fluorophenyl)carbamoyl)-2-fluoro-5-(((S)-1,1,1-trifluoropropan-2-yl)oxy)phenyl)-2-(hydroxymethyl)azetidine-1-carboxamide

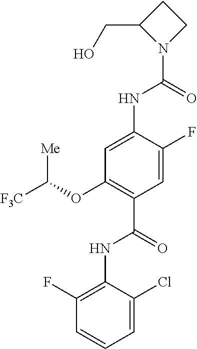 [SMILES: C[C@H](Oc1cc(NC(=O)N2CCC2CO)c(F)cc1C(=O)Nc1c(F)cccc1Cl)C(F)(F)F]

The title compound was prepared in a manner analogous to Example 1, however substituting azetidin-2-ylmethanol for N-ethylethanamine. MS (ESI): mass calcd. for C₂₁H₁₉ClF₅N₃O₄, 507.1, m/z found, 508.1 [M+H]⁺. ¹H NMR (400 MHz, CDCl₃) δ=9.13 (s, 1H), 8.33 (t, J=6.4 Hz, 1H), 7.98 (d, J=12.0 Hz, 1H), 7.32-7.29 (m, 1H), 7.27-7.20 (m, 1H), 7.16-7.09 (m, 1H), 5.08-4.95 (m, 1H), 4.66-4.55 (m, 1H), 4.19-4.09 (m, 1H), 4.00-3.87 (m, 3H), 3.31 (s, 1H), 2.40-2.28 (m, 1H), 2.05-1.94 (m, 1H), 1.67-1.63 (m, 3H).

### Example 13: N-(4-((2-Chloro-6-fluorophenyl)carbamoyl)-2-fluoro-5-(((S)-1,1,1-trifluoropropan-2-yl)oxy)phenyl)-3-(hydroxymethyl)pyrrolidine-1-carboxamide

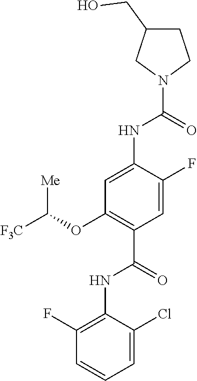 [SMILES: C[C@H](Oc1cc(NC(=O)N2CCC(CO)C2)c(F)cc1C(=O)Nc1c(F)cccc1Cl)C(F)(F)F]

The title compound was prepared in a manner analogous to Example 1, however substituting pyrrolidin-3-ylmethanol for N-ethylethanamine. MS (ESI): mass calcd. For C₂₂H₂₁ClF₅N₃O₄, 521.1; m/z found, 522.1 [M+H]⁺. ¹H NMR (400 MHz, CDCl₃) δ=9.07 (s, 1H), 8.32 (d, J=6.4 Hz, 1H), 8.03 (d, J=12.4 Hz, 1H), 7.28 (s, 1H), 7.24-7.18 (m, 1H), 7.14-7.07 (m, 1H), 6.75-6.69 (m, 1H), 5.09-4.91 (m, 1H), 3.80-3.62 (m, 4H), 3.56 (br s, 1H), 3.42-3.33 (m, 1H), 2.64-2.54 (m, 1H), 2.21-2.09 (m, 1H), 1.90-1.89 (m, 1H), 1.63 (d, J=6.4 Hz, 3H).

### Example 14: (S)-N-(2-Chloro-6-fluorophenyl)-5-fluoro-4-(3-methyl-3-propylureido)-2-((1,1,1-trifluoropropan-2-yl)oxy)benzamide

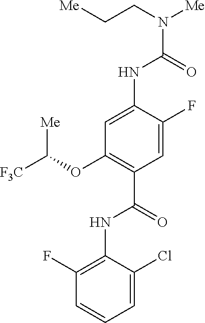 [SMILES: CCCN(C)C(=O)Nc1cc(O[C@@H](C)C(F)(F)F)c(C(=O)Nc2c(F)cccc2Cl)cc1F]

The title compound was prepared in a manner analogous to Example 1, however substituting N-methylpropan-1-amine for N-ethylethanamine. MS (ESI): mass calcd. For C₂₁H₂₁ClF₅N₃O₃, 493.1; m/z found, 494.1 [M+H]⁺. ¹H NMR (400 MHz, CDCl₃) δ=9.12 (s, 1H), 8.29 (d, J=6.4 Hz, 1H), 8.06 (d, J=12.4 Hz, 1H), 7.31 (t, J=1.2 Hz, 1H), 7.27-7.21 (m, 1H), 7.13 (ddd, J=1.6, 8.2, 9.6 Hz, 1H), 6.91 (d, J=4.4 Hz, 1H), 5.08-5.00 (m, 1H), 3.41-3.36 (m, 2H), 3.10 (s, 3H), 1.74-1.67 (m, 2H), 1.65 (d, J=6.4 Hz, 3H), 1.01 (t, J=7.2 Hz, 3H).

### Example 15: (S)-N-(2-Chloro-6-fluorophenyl)-4-(3-ethyl-3-propylureido)-5-fluoro-2-((1,1,1-trifluoropropan-2-yl)oxy)benzamide

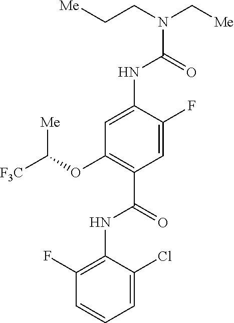 [SMILES: CCCN(CC)C(=O)Nc1cc(O[C@@H](C)C(F)(F)F)c(C(=O)Nc2c(F)cccc2Cl)cc1F]

The title compound was prepared in a manner analogous to Example 1, however substituting N-ethylpropan-1-amine for N-ethylethanamine. MS (ESI): mass calcd. For C₂₂H₂₃ClF₅N₃O₃, 507.1; m/z found, 508.1 [M+H]⁺. ¹H NMR (400 MHz, CDCl₃) δ=9.11 (s, 1H), 8.29 (d, J=6.4 Hz, 1H), 8.06 (d, J=12.4 Hz, 1H), 7.30 (s, 1H), 7.26-7.20 (m, 1H), 7.16-7.10 (m, 1H), 6.91 (d, J=4.8 Hz, 1H), 5.05 (td, J=6.0, 12.4 Hz, 1H), 3.48-3.42 (m, 2H), 3.33 (dd, J=6.8, 8.8 Hz, 2H), 1.76-1.68 (m, 2H), 1.65 (d, J=6.4 Hz, 3H), 1.30 (t, J=7.2 Hz, 3H), 1.02 (t, J=7.2 Hz, 3H).

### Example 16: (S)-N-(2-chloro-6-fluorophenyl)-5-fluoro-4-(3-(2-fluoroethyl)-3-methylureido)-2-((1,1,1-trifluoropropan-2-yl)oxy)benzamide

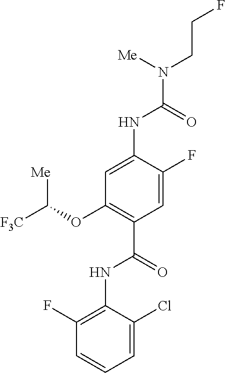 [SMILES: C[C@H](Oc1cc(NC(=O)N(C)CCF)c(F)cc1C(=O)Nc1c(F)cccc1Cl)C(F)(F)F]

The title compound was prepared in a manner analogous to Example 1, however substituting 2-fluoro-N-methyl-ethanamine for N-ethylethanamine. MS (ESI): mass calcd. for C₂₀H₁₈ClF₆N₃O₃, 497.1, m/z found, 498.1 [M+H]⁺. ¹H NMR (400 MHz, CDCl₃) δ=9.00 (s, 1H), 8.12 (d, J=6.0 Hz, 1H), 7.98 (d, J=11.2 Hz, 1H), 7.06-7.09 (m, 1H), 7.03-7.05 (m, 1H), 6.99-7.01 (m, 1H), 4.93-4.95 (m, 1H), 4.66 (t, J=3.2 Hz 1H), 4.54 (t, J=3.2 Hz 1H), 3.75-3.78 (m, 1H), 3.62-3.65 (m, 1H), 3.10 (s, 3H), 2.94-2.96 (m, 1H), 1.56 (d, J=6.4 Hz 3H).

### Example 17: (S)-N-(2-Chloro-6-fluorophenyl)-4-(3-(2-cyanoethyl)-3-methylureido)-5-fluoro-2-((1,1,1-trifluoropropan-2-yl)oxy)benzamide

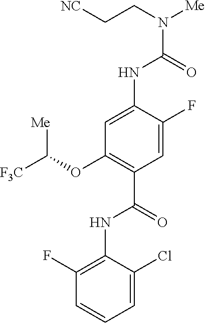 [SMILES: C[C@H](Oc1cc(NC(=O)N(C)CCC#N)c(F)cc1C(=O)Nc1c(F)cccc1Cl)C(F)(F)F]

The title compound was prepared in a manner analogous to Example 1, however substituting 3-(methylamino)propanenitrile for N-ethylethanamine. MS (ESI): mass calcd. For C₂₁H₁₈ClF₅N₄O₃, 504.1; m/z found, 505.1 [M+H]⁺. ¹H NMR (400 MHz, CDCl₃) δ=9.08 (s, 1H), 8.22 (d, J=6.4 Hz, 1H), 8.08 (d, J=12.0 Hz, 1H), 7.32-7.29 (m, 1H), 7.27-7.21 (m, 1H), 7.14 (dt, J=1.6, 8.8 Hz, 1H), 6.95 (d, J=4.0 Hz, 1H), 5.01 (td, J=6.4, 12.4 Hz, 1H), 3.75 (dt, J=2.8, 6.4 Hz, 2H), 3.30 (s, 3H), 2.75 (t, J=6.4 Hz, 2H), 1.67 (d, J=6.4 Hz, 3H).

### Example 18: (S)-N-(2-Chloro-6-fluorophenyl)-5-fluoro-4-(3-(2-hydroxyethyl)-3-methylureido)-2-((1,1,1-trifluoropropan-2-yl)oxy)benzamide

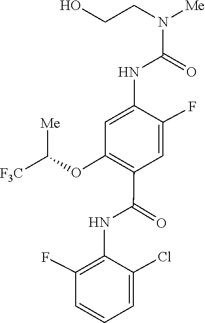 [SMILES: C[C@H](Oc1cc(NC(=O)N(C)CCO)c(F)cc1C(=O)Nc1c(F)cccc1Cl)C(F)(F)F]

The title compound was prepared in a manner analogous to Example 1, however substituting 2-(methylamino)ethanol for N-ethylethanamine. MS (ESI): mass calcd. For C₂₀H₁₉ClF₅N₃O₄, 495.1; m/z found, 496.1 [M+H]⁺. ¹H NMR (400 MHz, CDCl₃) δ=9.11 (s, 1H), 8.54-8.28 (m, 1H), 8.15 (d, J=6.4 Hz, 1H), 8.02 (d, J=12.0 Hz, 1H), 7.30 (br d, J=1.2 Hz, 1H), 7.26-7.20 (m, 1H), 7.15-7.10 (m, 1H), 5.02 (td, J=6.4, 12.4 Hz, 1H), 3.96 (t, J=4.4 Hz, 2H), 3.65-3.52 (m, 2H), 3.11 (s, 3H), 2.50-2.37 (m, 1H), 1.65 (d, J=6.4 Hz, 3H).

### Example 19: (S)-N-(4-((2-Chloro-6-fluorophenyl)carbamoyl)-2-fluoro-5-((1,1,1-trifluoropropan-2-yl)oxy)phenyl)pyrrolidine-1-carboxamide

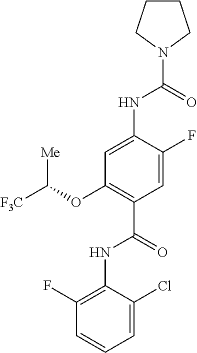 [SMILES: C[C@H](Oc1cc(NC(=O)N2CCCC2)c(F)cc1C(=O)Nc1c(F)cccc1Cl)C(F)(F)F]

The title compound was prepared in a manner analogous to Example 1, however substituting pyrrolidine for N-ethylethanamine. MS (ESI): mass calcd. For C₂₁H₁₉ClF₅N₃O₃, 491.1; m/z found, 492.1 [M+H]⁺. ¹H NMR (400 MHz, CDCl₃) δ=9.08 (s, 1H), 8.33 (d, J=6.4 Hz, 1H), 8.03 (d, J=12.4 Hz, 1H), 7.28 (s, 1H), 7.24-7.18 (m, 1H), 7.13-7.08 (m, 1H), 6.71 (d, J=4.4 Hz, 1H), 5.01 (td, J=6.4, 12.4 Hz, 1H), 3.52 (t, J=6.4 Hz, 4H), 2.03 (s, 4H), 1.63 (d, J=6.4 Hz, 1H).

### Example 20: (S)-N-(4-((2-Chloro-6-fluorophenyl)carbamoyl)-2-fluoro-5-((1,1,1-trifluoropropan-2-yl)oxy)phenyl)azetidine-1-carboxamide

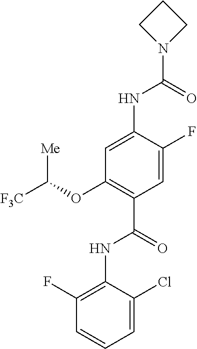 [SMILES: C[C@H](Oc1cc(NC(=O)N2CCC2)c(F)cc1C(=O)Nc1c(F)cccc1Cl)C(F)(F)F]

The title compound was prepared in a manner analogous to Example 1, however substituting azetidine for N-ethylethanamine. MS (ESI): mass calcd. for C₂₀H₁₇ClF₅N₃O₃, 477.1, m/z found, 478.1 [M+H]⁺. ¹H NMR (400 MHz, CDCl₃) δ=9.07 (s, 1H), 8.28 (d, J=6.4 Hz, 1H), 8.03 (d, J=12.0 Hz, 1H), 7.29 (t, J=1.2 Hz, 1H), 7.25-7.18 (m, 1H), 7.15-7.08 (m, 1H), 6.42-6.36 (m, 1H), 5.06-4.91 (m, 1H), 4.17 (t, J=7.6 Hz, 4H), 2.45-2.35 (m, 2H), 1.64 (d, J=6.4 Hz, 3H).

### Example 21: N-(4-((2-Chloro-6-fluorophenyl)carbamoyl)-2-fluoro-5-(((S)-1,1,1-trifluoropropan-2-yl)oxy)phenyl)-2-(hydroxymethyl)pyrrolidine-1-carboxamide

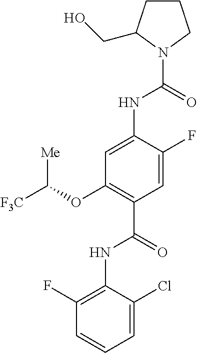 [SMILES: C[C@H](Oc1cc(NC(=O)N2CCCC2CO)c(F)cc1C(=O)Nc1c(F)cccc1Cl)C(F)(F)F]

The title compound was prepared in a manner analogous to Example 1, however substituting pyrrolidin-2-ylmethanol for N-ethylethanamine. MS (ESI): mass calcd. for C₂₂H₂₁ClF₅N₃O₄, 521.1; m/z found, 522.1 [M+H]⁺. ¹H NMR (400 MHz, CDCl₃) δ=9.12 (s, 1H), 8.25 (dd, J=6.4, 11.2 Hz, 1H), 8.03 (d, J=11.2 Hz, 1H), 7.27-7.29 (m, 1H), 7.13-7.15 (m, 1H), 7.11-7.12 (m, 1H), 5.10-5.12 (m, 1H), 4.17-4.19 (m, 1H), 3.73-3.72 (m, 1H), 3.69-3.71 (m, 2H), 3.51-3.53 (m, 1H), 2.15-2.16 (m, 1H), 1.99-2.01 (m, 2H), 1.66-1.68 (m, 1H), 1.65 (t, J=6.4, 11.2 Hz, 3H).

### Example 22: (S)-N-(4-((2-Chloro-6-fluorophenyl)carbamoyl)-2-fluoro-5-((1,1,1-trifluoropropan-2-yl)oxy)phenyl)-3-fluoroazetidine-1-carboxamide

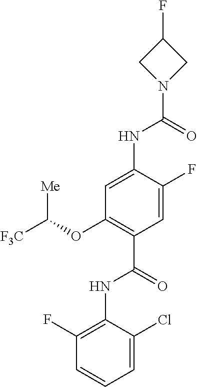 [SMILES: C[C@H](Oc1cc(NC(=O)N2CC(F)C2)c(F)cc1C(=O)Nc1c(F)cccc1Cl)C(F)(F)F]

The title compound was prepared in a manner analogous to Example 1, however substituting 3-fluoroazetidine for N-ethylethanamine. MS (ESI): mass calcd. For C₂₀H₁₆ClF₆N₃O₃, 495.1; m/z found, 496.1 [M+H]⁺. ¹H NMR (400 MHz, CDCl₃) δ=9.08 (s, 1H), 8.25 (d, J=6.4 Hz, 1H), 8.07 (d, J=12.4 Hz, 1H), 7.31-7.29 (m, 1H), 7.27-7.21 (m, 1H), 7.16-7.10 (m, 1H), 6.46 (br d, J=4.0 Hz, 1H), 5.52-5.32 (m, 1H), 5.00 (td, J=6.4, 12.4 Hz, 1H), 4.49-4.39 (m, 2H), 4.36-4.25 (m, 2H), 1.66 (d, J=6.4 Hz, 3H).

### Example 23: (S)-N-(4-((2-Chloro-6-fluorophenyl)carbamoyl)-2-fluoro-5-((1,1,1-trifluoropropan-2-yl)oxy)phenyl)-3-hydroxyazetidine-1-carboxamide

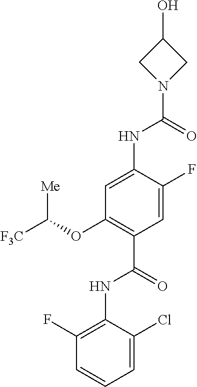 [SMILES: C[C@H](Oc1cc(NC(=O)N2CC(O)C2)c(F)cc1C(=O)Nc1c(F)cccc1Cl)C(F)(F)F]

The title compound was prepared in a manner analogous to Example 1, however substituting azetidin-3-ol for N-ethylethanamine. MS (ESI): mass calcd. For C₂₀H₁₇ClF₅N₃O₄, 493.1; m/z found, 494.1 [M+H]⁺. ¹H NMR (400 MHz, CDCl₃) δ=9.08 (s, 1H), 8.27 (d, J=6.4 Hz, 1H), 8.05 (d, J=12.4 Hz, 1H), 7.30 (d, J=1.2 Hz, 1H), 7.27-7.21 (m, 1H), 7.13 (dt, J=1.2, 8.8 Hz, 1H), 6.45 (d, J=3.6 Hz, 1H), 5.00 (td, J=6.0, 12.4 Hz, 1H), 4.83-4.77 (m, 1H), 4.39 (dd, J=7.2, 8.4 Hz, 2H), 4.05 (dd, J=4.4, 8.8 Hz, 2H), 2.25 (d, J=5.6 Hz, 1H), 1.65 (d, J=6.4 Hz, 3H).

### Example 24: (S)-N-(4-((2-Chloro-6-fluorophenyl)carbamoyl)-2-fluoro-5-((1,1,1-trifluoropropan-2-yl)oxy)phenyl)-3-(hydroxymethyl)azetidine-1-carboxamide

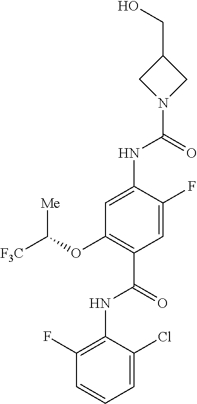 [SMILES: C[C@H](Oc1cc(NC(=O)N2CC(CO)C2)c(F)cc1C(=O)Nc1c(F)cccc1Cl)C(F)(F)F]

The title compound was prepared in a manner analogous to Example 1, however substituting azetidin-3-ylmethanol for N-ethylethanamine. MS (ESI): C₂₁H₁₉ClF₅N₃O₄, 507.1; m/z found, 508.1 [M+H]⁺. ¹H NMR (400 MHz, CDCl₃) δ=9.12 (s, 1H), 8.28 (d, J=6 Hz, 1H), 8.03 (d, J=11.2 Hz, 1H), 7.29-7.31 (m, 1H), 7.12-7.15 (m, 1H), 7.10-7.11 (m, 1H), 6.43 (s, 1H), 5.01-5.03 (m, 1H), 4.25-4.27 (m, 2H), 3.97-4.00 (m, 2H), 3.86-3.89 (m, 2H), 2.94-2.96 (m, 1H), 1.65 (d, J=6.4 Hz 1H), 1.58-1.60 (m, 1H).

### Example 25: (S)-N-(4-((2-Chloro-6-fluorophenyl)carbamoyl)-2-fluoro-5-((1,1,1-trifluoropropan-2-yl)oxy)phenyl)-3-(fluoromethyl)azetidine-1-carboxamide

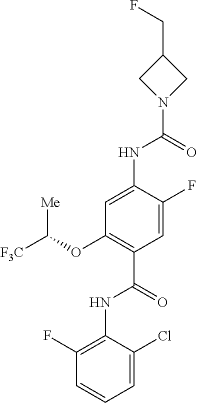 [SMILES: C[C@H](Oc1cc(NC(=O)N2CC(CF)C2)c(F)cc1C(=O)Nc1c(F)cccc1Cl)C(F)(F)F]

To a solution of (S)-N-(4-((2-chloro-6-fluorophenyl)carbamoyl)-2-fluoro-5-((1,1,1-trifluoropropan-2-yl)oxy)phenyl)-3-(hydroxymethyl)azetidine-1-carboxamide (Example 24, 0.1 g, 197 μmol) in DCM (10 mL) was added dropwise DAST (159 mg, 985 μmol, 130 μL) at −78° C. The mixture was stirred at −78° C. for 0.5 hr then warmed to room temperature and stirred for 2.5 hr. The reaction mixture was quenched with aqueous sat. NaHCO₃ (10 mL). The mixture was extracted with EtOAc (30 mL×3) and the combined organic layer was dried over reduced pressure. The crude product was purified by prep-HPLC (method A) to give the title compound as a white solid. MS (ESI): mass calcd. for C₂₁H₁₈ClF₆N₃O₃, 509.1 m/z found, 510.1 [M+H]⁺. ¹H NMR (400 MHz, CDCl₃) δ=9.08 (s, 1H), 8.28 (d, J=6.0 Hz, 1H), 8.07 (d, J=11.2 Hz, 1H), 7.26-7.30 (m, 1H), 7.22-7.24 (m, 1H), 7.12-7.15 (m, 1H), 6.43-6.44 (m, 1H), 5.00-5.03 (m, 1H), 4.69 (d, J=3.2 Hz 1H), 4.57 (d, J=3.2 Hz 1H), 4.24-4.26 (m, 2H), 4.03-4.05 (m, 2H), 3.10-3.05 (m, 1H), 1.66 (d, J=6.4 Hz 3H).

### Example 26: (S)-N-(2-Chloro-6-fluorophenyl)-4-(3-(cyanomethyl)-3-methylureido)-5-fluoro-2-((1,1,1-trifluoropropan-2-yl)oxy)benzamide

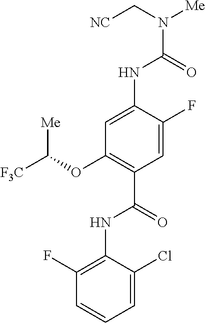 [SMILES: C[C@H](Oc1cc(NC(=O)N(C)CC#N)c(F)cc1C(=O)Nc1c(F)cccc1Cl)C(F)(F)F]

The title compound was prepared in a manner analogous to Example 1, however substituting 2-(methylamino)acetonitrile for N-ethylethanamine. MS (ESI): mass calcd. For C₂₀H₁₆ClF₄N₅O₃, 490.1; m/z found, 491.1 [M+H]⁺. ¹H NMR (400 MHz, CDCl₃) δ=8.99 (s, 1H), 8.21 (d, J=10.4 Hz, 1H), 7.33-7.30 (m, 2H), 7.28-7.24 (m, 1H), 7.18-7.12 (m, 1H), 7.10 (d, J=5.5 Hz, 1H), 4.91 (td, J=6.0, 12.4 Hz, 1H), 4.24 (s, 2H), 3.09 (s, 3H), 1.67 (d, J=6.4 Hz, 3H).

### Example 27: (R)-N-(4-((2-Chloro-6-fluorophenyl)carbamoyl)-2-fluoro-5-(((S)-1,1,1-trifluoropropan-2-yl)oxy)phenyl)-2-(hydroxymethyl)pyrrolidine-1-carboxamide

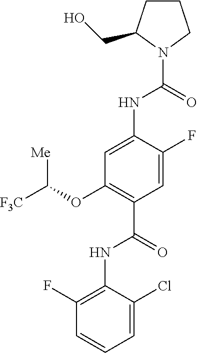 [SMILES: C[C@H](Oc1cc(NC(=O)N2CCC[C@@H]2CO)c(F)cc1C(=O)Nc1c(F)cccc1Cl)C(F)(F)F]

The title compound was prepared in a manner analogous to Example 1, however substituting (R)-pyrrolidin-2-ylmethanol for N-ethylethanamine. MS (ESI): mass calcd. For C₂₂H₂₁ClF₅N₃O₄, 521.1; m/z found, 522.1 [M+H]⁺. ¹H NMR (400 MHz, CDCl₃) δ=9.09 (s, 1H), 8.22 (d, J=6.4 Hz, 1H), 7.99 (d, J=12.0 Hz, 1H), 7.28 (s, 1H), 7.21 (dt, J=5.6, 8.0 Hz, 1H), 7.13-7.07 (m, 1H), 5.03-4.97 (m, 1H), 4.15 (br t, J=8.0 Hz, 1H), 3.84-3.78 (m, 1H), 3.76-3.66 (m, 2H), 3.53-3.46 (m, 1H), 2.17-2.07 (m, 1H), 2.02-1.91 (m, 2H), 1.76-1.67 (m, 1H), 1.63 (d, J=6.4 Hz, 3H).

### Example 28: (S)-N-(4-((2-Chloro-6-fluorophenyl)carbamoyl)-2-fluoro-5-(((S)-1,1,1-trifluoropropan-2-yl)oxy)phenyl)-2-(hydroxymethyl)pyrrolidine-1-carboxamide

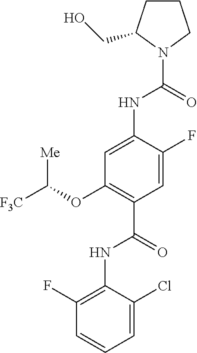 [SMILES: C[C@H](Oc1cc(NC(=O)N2CCC[C@H]2CO)c(F)cc1C(=O)Nc1c(F)cccc1Cl)C(F)(F)F]

The title compound was prepared in a manner analogous to Example 1, however substituting (S)-pyrrolidin-2-ylmethanol for N-ethylethanamine. MS (ESI): mass calcd. For C₂₂H₂₁ClF₅N₃O₄, 521.1; m/z found, 522.1 [M+H]⁺. ¹H NMR (400 MHz, CDCl₃) δ=9.12 (s, 1H), 8.25 (d, J=6.4 Hz, 1H), 8.00 (d, J=12.0 Hz, 1H), 7.29-2.27 (m, 1H), 7.26-7.18 (m, 1H), 7.15-7.08 (m, 1H), 5.05-4.96 (m, 1H), 4.16 (br s, 1H), 3.84-3.77 (m, 1H), 3.77-3.62 (m, 2H), 3.51 (s, 1H), 2.19-2.04 (m, 1H), 1.98 (s, 2H), 1.79-1.66 (m, 1H), 1.63 (d, J=6.4 Hz, 3H).

### Example 29: N-(4-((2-Chloro-6-fluorophenyl)carbamoyl)-2-fluoro-5-(((S)-1,1,1-trifluoropropan-2-yl)oxy)phenyl)-3-methoxypyrrolidine-1-carboxamide

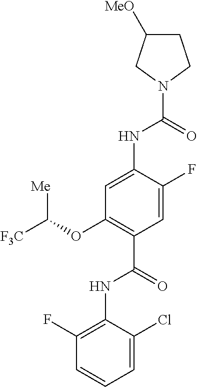 [SMILES: COC1CCN(C(=O)Nc2cc(O[C@@H](C)C(F)(F)F)c(C(=O)Nc3c(F)cccc3Cl)cc2F)C1]

The title compound was prepared in a manner analogous to Example 1, however substituting 3-methoxypyrrolidine for N-ethylethanamine. MS (ESI): mass calcd. For C₂₂H₂₁ClF₅N₃O₄, 521.1; m/z found, 522.1 [M+H]⁺. ¹H NMR (400 MHz, CDCl₃) δ=9.08 (s, 1H), 8.31 (dd, J=2.0, 6.4 Hz, 1H), 8.04 (d, J=12.0 Hz, 1H), 7.31-7.27 (m, 1H), 7.25-7.19 (m, 1H), 7.15-7.07 (m, 1H), 6.70 (d, J=4.4 Hz, 1H), 5.02-4.99 (m, 1H), 4.07 (s, 1H), 3.70-3.55 (m, 4H), 3.38 (s, 3H), 2.33-1.95 (m, 2H), 1.64 (d, J=6.4 Hz, 3H).

### Example 30: N-(4-((2-Chloro-6-fluorophenyl)carbamoyl)-2-fluoro-5-(((S)-1,1,1-trifluoropropan-2-yl)oxy)phenyl)-3-hydroxypyrrolidine-1-carboxamide

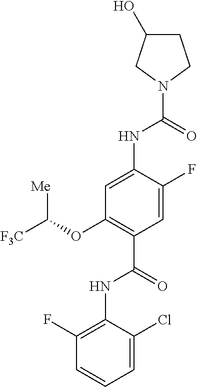 [SMILES: C[C@H](Oc1cc(NC(=O)N2CCC(O)C2)c(F)cc1C(=O)Nc1c(F)cccc1Cl)C(F)(F)F]

The title compound was prepared in a manner analogous to Example 1, however substituting pyrrolidin-3-ol for N-ethylethanamine. MS (ESI): mass calcd. For C₂₁H₁₉ClF₅N₃O₄, 507.1; m/z found, 508.1 [M+H]⁺. ¹H NMR (400 MHz, CDCl₃) δ=9.07 (s, 1H), 8.30 (dd, J=1.6, 6.4 Hz, 1H), 8.03 (d, J=12.0 Hz, 1H), 7.28 (t, J=1.2 Hz, 1H), 7.21 (dt, J=5.6, 8.0 Hz, 1H), 7.13-7.08 (m, 1H), 6.70 (d, J=4.0 Hz, 1H), 5.04-4.97 (m, 1H), 4.63 (s, 1H), 3.73-3.55 (m, 4H), 2.20-2.07 (m, 2H), 1.69 (d, J=1.6 Hz, 1H), 1.63 (d, J=6.4 Hz, 3H).

### Example 31: N-(4-((2-Chloro-6-fluorophenyl)carbamoyl)-2-fluoro-5-(((S)-1,1,1-trifluoropropan-2-yl)oxy)phenyl)-2-(methoxymethyl)pyrrolidine-1-carboxamide

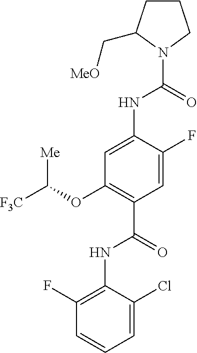 [SMILES: COCC1CCCN1C(=O)Nc1cc(O[C@@H](C)C(F)(F)F)c(C(=O)Nc2c(F)cccc2Cl)cc1F]

The title compound was prepared in a manner analogous to Example 1, however substituting 2-(methoxymethyl)pyrrolidine for N-ethylethanamine. MS (ESI): mass calcd. For C₂₃H₂₃ClF₅N₃O₄, 535.1; m/z found, 536.1 [M+H]⁺. ¹H NMR (400 MHz, CDCl₃) δ=9.59-9.27 (m, 1H), 9.09 (d, J=6.4 Hz, 1H), 8.21 (dd, J=6.4, 19.6 Hz, 1H), 7.99 (d, J=12.0 Hz, 1H), 7.28 (s, 1H), 7.20 (dt, J=5.6, 8.4 Hz, 1H), 7.13-7.07 (m, 1H), 5.05-4.96 (m, 1H), 4.09 (t, J=9.2 Hz, 1H), 3.84-3.75 (m, 1H), 3.55 (dd, J=2.4, 9.2 Hz, 1H), 3.49 (d, J=1.6 Hz, 3H), 3.47-3.40 (m, 2H), 2.19-2.09 (m, 1H), 1.97-1.83 (m, 2H), 1.72-1.68 (m, 1H), 1.62 (dd, J=6.4, 12.0 Hz, 3H).

### Example 32: N-(4-((2-Chloro-6-fluorophenyl)carbamoyl)-2-fluoro-5-(((S)-1,1,1-trifluoropropan-2-yl)oxy)phenyl)-3-fluoropyrrolidine-1-carboxamide

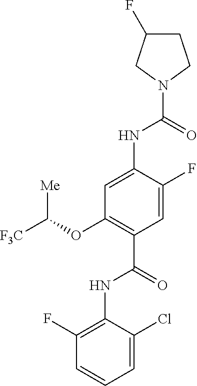 [SMILES: C[C@H](Oc1cc(NC(=O)N2CCC(F)C2)c(F)cc1C(=O)Nc1c(F)cccc1Cl)C(F)(F)F]

The title compound was prepared in a manner analogous to Example 1, however substituting 3-fluoropyrrolidine for N-ethylethanamine. MS (ESI): mass calcd. For C₂₁H₁₈ClF₆N₃O₃, 509.1; m/z found, 510.1 [M+H]⁺. ¹H NMR (400 MHz, CDCl₃) δ=9.07 (s, 1H), 8.29 (dd, J=2.0, 6.4 Hz, 1H), 8.04 (d, J=12.0 Hz, 1H), 7.29-7.26 (m, 1H), 7.25-7.18 (m, 1H), 7.14-7.08 (m, 1H), 6.70 (br d, J=4.4 Hz, 1H), 5.43-5.27 (m, 1H), 5.00 (td, J=6.4, 12.4 Hz, 1H), 3.95-3.84 (m, 1H), 3.75-3.61 (m, 3H), 2.49-2.37 (m, 1H), 2.28-2.07 (m, 1H), 1.65-1.62 (m, 3H).

### Example 33: (S)-N-(4-((2-Chloro-6-fluorophenyl)carbamoyl)-2-fluoro-5-((1,1,1-trifluoropropan-2-yl)oxy)phenyl)-3,3-difluoropyrrolidine-1-carboxamide

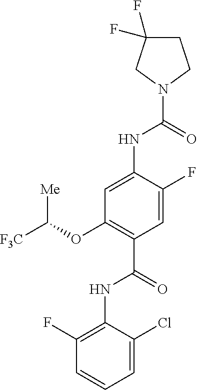 [SMILES: C[C@H](Oc1cc(NC(=O)N2CCC(F)(F)C2)c(F)cc1C(=O)Nc1c(F)cccc1Cl)C(F)(F)F]

The title compound was prepared in a manner analogous to Example 1, however substituting 3,3-difluoropyrrolidine hydrochloride for N-ethylethanamine. MS (ESI): mass calcd. For C₂₁H₁₇ClF₇N₃O₃, 527.1; m/z found, 528.1 [M+H]⁺. ¹H NMR (400 MHz, CDCl₃) δ=9.08 (s, 1H), 8.24 (d, J=6.4 Hz, 1H), 8.06 (d, J=12.0 Hz, 1H), 7.29-7.27 (m, 1H), 7.26-7.19 (m, 1H), 7.16-7.09 (m, 1H), 6.66 (d, J=4.0 Hz, 1H), 5.04-4.95 (m, 1H), 3.89 (t, J=12.4 Hz, 2H), 3.78 (t, J=7.2 Hz, 2H), 2.60-2.47 (m, 2H), 1.65 (d, J=6.4 Hz, 3H).

### Example 34: N-(4-((2-Chloro-6-fluorophenyl)carbamoyl)-2-fluoro-5-(((S)-1,1,1-trifluoropropan-2-yl)oxy)phenyl)-2-(hydroxymethyl)-5-oxopyrrolidine-1-carboxamide

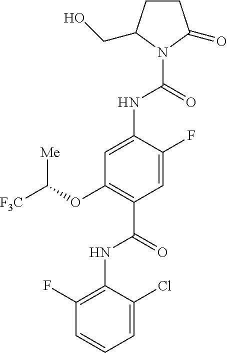 [SMILES: C[C@H](Oc1cc(NC(=O)N2C(=O)CCC2CO)c(F)cc1C(=O)Nc1c(F)cccc1Cl)C(F)(F)F]

Step A. 5-(((tert-Butyldimethylsilyl)oxy)methyl)pyrrolidin-2-one. To a solution of 5-(hydroxymethyl)pyrrolidin-2-one (500 mg, 4.34 mmol) in DCM (5 mL) were added TBSCl (785 mg, 5.21 mmol, 639 μL) and imidazole (443 mg, 6.51 mmol). The mixture was stirred at 15° C. for 4 hr. The mixture was poured into water (50 mL). The aqueous phase was extracted with ethyl acetate (30 mL×2). The combined organic layers were washed with brine (20 mL×2), dried over Na₂SO₄, filtered and concentrated in vacuo. The residue was purified by column chromatography (SiO₂, Petroleum ether/Ethyl acetate=10/1 to 2/1) to give the title compound as yellow oil.

Step B. 2-(((tert-Butyldimethylsilyl)oxy)methyl)-N-(4-((2-chloro-6-fluorophenyl)carbamoyl)-2-fluoro-5-(((S)-1,1,1-trifluoropropan-2-yl)oxy)phenyl)-5-oxopyrrolidine-1-carboxamide. To a solution of 5-(((tert-butyldimethylsilyl)oxy)methyl)pyrrolidin-2-one (446 mg, 1.94 mmol, 22.7 μL) in THF (3 mL) was added NaH (77.7 mg, 1.94 mmol, 60% purity) at 0° C. and the mixture was stirred at 0° C. for 1 h. Then (S)-phenyl (4-((2-chloro-6-fluorophenyl)carbamoyl)-2-fluoro-5-((1,1,1-trifluoropropan-2-yl)oxy)phenyl)carbamate (Intermediate 1, 100 mg, 194 μmol) in THE (1 mL) was added and the mixture stirred at 15° C. for 2 hr. The mixture was poured into sat. NH₄Cl solution (20 mL). The aqueous phase was extracted with ethyl acetate (20 mL×2). The combined organic layers were washed with brine (10 mL×2), dried over Na₂SO₄, filtered and concentrated in vacuo. The residue was purified by column chromatography (SiO₂, Petroleum ether/Ethyl acetate=1/0 to 5/1) to give the title compound as a yellow oil.

Step C. N-(4-((2-Chloro-6-fluorophenyl)carbamoyl)-2-fluoro-5-(((S)-1,1,1-trifluoropropan-2-yl)oxy)phenyl)-2-(hydroxymethyl)-5-oxopyrrolidine-1-carboxamide. To a solution of 2-(((tert-butyldimethylsilyl)oxy)methyl)-N-(4-((2-chloro-6-fluorophenyl)carbamoyl)-2-fluoro-5-(((S)-1,1,1-trifluoropropan-2-yl)oxy)phenyl)-5-oxopyrrolidine-1-carboxamide (100 mg, 154 μmol) in THE (2 mL) was added TBAF (1 M, 231 μL) at 0° C. The mixture was stirred at 15° C. for 1 hr. The mixture was poured into water (20 mL). The aqueous phase was extracted with ethyl acetate (20 mL×2). The combined organic layers were washed with brine (10 mL×2), dried over Na₂SO₄, filtered and concentrated in vacuo. The residue was purified by prep-HPLC (method A) to give the title compound as a white solid. MS (ESI): mass calcd. For C₂₂H₁₉ClF₅N₃O₅, 535.1; m/z found, 536.1 [M+H]⁺. ¹H NMR (400 MHz, CDCl₃) δ=11.41 (d, J=2.4 Hz, 1H), 9.06 (s, 1H), 8.20 (d, J=6.0 Hz, 1H), 8.07 (d, J=11.6 Hz, 1H), 7.30-7.27 (m, 1H), 7.25-7.19 (m, 1H), 7.14-7.09 (m, 1H), 5.02-4.92 (m, 1H), 4.55 (qd, J=3.2, 9.2 Hz, 1H), 4.04 (ddd, J=3.2, 5.6, 11.6 Hz, 1H), 3.82 (td, J=4.4, 11.6 Hz, 1H), 2.94 (td, J=9.6, 18.4 Hz, 1H), 2.67-2.58 (m, 1H), 2.44 (t, J=5.6 Hz, 1H), 2.29 (qd, J=9.6, 13.2 Hz, 1H), 2.06 (tdd, J=2.8, 10.0, 12.8 Hz, 1H), 1.65 (d, J=6.4 Hz, 3H).

### Example 35: (S)-N-(2-Chloro-6-fluorophenyl)-4-(3,3-diethyl-1-methylureido)-5-fluoro-2-((1,1,1-trifluoropropan-2-yl)oxy)benzamide

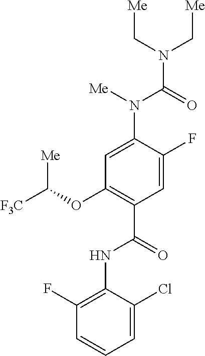 [SMILES: CCN(CC)C(=O)N(C)c1cc(O[C@@H](C)C(F)(F)F)c(C(=O)Nc2c(F)cccc2Cl)cc1F]

Step A. (S)-Methyl 4-(3,3-diethylureido)-5-fluoro-2-((1,1,1-trifluoropropan-2-yl)oxy)benzoate. To a solution of diethylamine (82.01 mg, 1.12 mmol) in DCM (2 mL) was added TEA (226.94 mg, 2.24 mmol). Then a solution of methyl (S)-5-fluoro-4-((phenoxycarbonyl)amino)-2-((1,1,1-trifluoropropan-2-yl)oxy)benzoate (Intermediate 2, 300 mg, 748 μmol) in DCM (1 mL) was added dropwise. The resulting mixture was stirred at 30° C. for 2 hr. The reaction mixture was diluted with H₂O (10 mL) and extracted with DCM (20 mL×3). The combined organic layers were washed with brine (40 mL), dried over Na₂SO₄, filtered and concentrated in vacuo. The residue was purified by column chromatography (petroleum ether/ethyl acetate=20/1 to 7/1) to give the title compound as a colorless oil. MS (ESI): mass calcd. for C₁₆H₂₀F₄N₂O₄, 380.1; m/z found, 381.3 [M+H]⁺.

Step B. (S)-Methyl 4-(3,3-diethyl-1-methylureido)-5-fluoro-2-((1,1,1-trifluoropropan-2-yl)oxy)benzoate. To a mixture of (S)-methyl 4-(3,3-diethylureido)-5-fluoro-2-((1,1,1-trifluoro propan-2-yl)oxy)benzoate (50 mg, 131 μmol) in DMF (3 mL) was added NaH (7.89 mg, 197 μmol) in one portion at 0° C. under N2. The mixture was stirred at 0° C. for 30 mins, and then added Mel (22.49 mg, 158 μmol). The mixture was stirred at 20° C. for 1.5 hours. The reaction mixture was quenched by addition HCl (1M) at 0° C. to adjust pH to 6, and then diluted with H₂O (10 mL) and extracted with ethyl acetate (10 mL×3). The combined organic layers were washed with brine (30 mL), dried over Na₂SO₄, filtered and concentrated in vacuo. The residue was purified by prep-TLC (SiO₂, petroleum ether/ethyl acetate=3/1) to give the title compound as a yellow solid. MS (ESI): mass calcd. for C₁₇H₂₂F₄N₂O₄, 394.2; m/z found, 395.4 [M+H]⁺. ¹H NMR (400 MHz, CDCl₃) δ=7.66 (d, J=11.2 Hz, 1H), 6.80 (d, J=6.8 Hz, 1H), 4.62-4.50 (m, 1H), 3.92 (s, 3H), 3.20-3.14 (m, 7H), 1.58-1.53 (m, 6H), 1.01 (t, J=7.2 Hz, 6H).

Step C. (S)-N-(2-Chloro-6-fluorophenyl)-4-(3,3-diethyl-1-methylureido)-5-fluoro-2-((1,1,1-trifluoropropan-2-yl)oxy)benzamide. To a solution of 2-chloro-6-fluoroaniline (44.3 mg, 304 μmol) in DCM (2 mL) was added dropwise AlMe₃ (2 M, 203 μL) at 25° C. The mixture was stirred at 25° C. for 0.5 hr. Then the solution of (S)-methyl 4-(3,3-diethyl-1-methylureido)-5-fluoro-2-((1,1,1-trifluoropropan-2-yl)oxy)benzoate (40 mg, 101 μmol) in DCM (1 mL) was added and the resulting mixture was heated to reflux and stirred at 60° C. for 12 hr. The reaction mixture was quenched with HCl (1M) to adjust pH to 6, then diluted with H₂O (10 mL) and extracted with DCM (10 mL×2). The combined organic layers were washed with brine (30 mL), dried over Na₂SO₄, filtered and concentrated in vacuo. The residue was purified by by prep-HPLC (method A) to give the title compound as a yellow solid. MS (ESI): mass calcd. for C₂₂H₂₃ClF₅N₃O₃, 507.1; m/z found, 508.2 [M+H]⁺. ¹H NMR (400 MHz, CDCl₃) δ=8.96 (s, 1H), 8.09 (d, J=11.6 Hz, 1H), 7.31-7.28 (m, 1H), 7.25-7.21 (m, 1H), 7.16-7.10 (m, 1H), 6.77 (d, J=6.0 Hz, 1H), 4.94-4.71 (m, 1H), 3.22-3.15 (m, 7H), 1.62 (d, J=6.4 Hz, 3H), 1.03 (t, J=7.2 Hz, 6H).

### Example 36: (R)-N-(4-((2-Chloro-4-methylpyridin-3-yl)carbamoyl)-2-fluoro-5-(((S)-1,1,1-trifluoropropan-2-yl)oxy)phenyl)-2-(hydroxymethyl)pyrrolidine-1-carboxamide

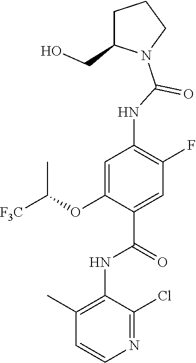 [SMILES: Cc1ccnc(Cl)c1NC(=O)c1cc(F)c(NC(=O)N2CCC[C@@H]2CO)cc1O[C@@H](C)C(F)(F)F]

Step A. (S)-4-((Diphenylmethylene)amino)-5-fluoro-2-((1,1,1-trifluoropropan-2-yl)oxy)benzoic acid. LiOH.H₂O (247 mg, 5.90 mmol) was slowly added into the solution of (S)-methyl 4-((diphenylmethylene)amino)-5-fluoro-2-((1,1,1-trifluoropropan-2-yl)oxy)benzoate (Intermediate 6, 2 g, 2.95 mmol) in THF/120 (v/v, 3/1, 4 mL) at room temperature. The reaction was heated to 40° C. for 16 hours. The mixture was diluted by ethyl acetate (30 mL) and water (10 mL), adjusted the pH to 6 by 1 M aq. HCl. The organic layer was washed by brine (10 mL), dried by Na₂SO₄, filtered and concentrated. The residue was purified by column chromatography (SiO₂, gradient elution: 0-60% ethyl acetate in petroleum ether) to give the title compound as a yellow solid. MS (ESI): mass calcd. For C₂₃H₁₇F₄NO₃, 431.1; m/z found, 432.1 [M+H]⁺. ¹H NMR (400 MHz, CDCl₃) δ=7.75 (s, 2H), 7.70 (d, J=10.4 Hz, 1H), 7.27-7.56 (m, 6H), 7.14 (s, 2H), 6.38 (d, J=6.0 Hz, 1H), 4.55 (dt, J=12.1 Hz, 1H), 1.42 (d, J=6.4 Hz, 3H); ¹⁹F NMR (376 MHz, CDCl₃) δ=−78.43 (br d, J=6.6 Hz, 1F), −132.86-−126.72 (m, 1F).

Step B. (S)-N-(2-Chloro-4-methylpyridin-3-yl)-4-((diphenylmethylene)amino)-5-fluoro-2-((1,1,1-trifluoropropan-2-yl)oxy)benzamide. POCl₃ (295 mg, 1.9 mmol) was slowly added to a mixture of (S)-4-((diphenylmethylene)amino)-5-fluoro-2-((1,1,1-trifluoropropan-2-yl)oxy)benzoic acid (600 mg, 963 μmol, 69% purity), 2-chloro-4-methylpyridin-3-amine (137 mg, 963 μmol), pyridine (381 mg, 4.8 mmol) in DCM (5 mL) at room temperature. The reaction mixture was stirred at room temperature for 2 hours. The reaction mixture was quenched with sat. aq. K₂CO₃ (10 mL) and extracted with DCM (20 mL×3). The organic layer was separated and concentrated. The residue was purified by column chromatography (SiO₂, gradient elution: 0-60% ethyl acetate in petroleum ether) to give the title compound as yellow oil. MS (ESI): mass calcd. for C₂₉H₂₂ClF₄N₃O₂, 555.1; m/z found, 556.1 [M+H]⁺. ¹H NMR (400 MHz, CDCl₃) δ=8.97 (s, 1H), 8.11 (d, J=5.1 Hz, 1H), 7.72 (d, J=7.5 Hz, 2H), 7.46 (d, J=8.4 Hz, 1H), 7.34-7.42 (m, 2H), 7.29 (d, J=7.3 Hz, 3H), 7.11 (dd, J=13.1, 4H), 6.33 (d, J=6.2 Hz, 1H), 4.51-4.62 (m, 1H), 2.24 (s, 3H), 1.35-1.46 (m, 3H); ¹⁹F NMR (376 MHz, CDCl₃) δ=−78.23 (br d, J=6.6 Hz, 1F), −134.34-−127.65 (m, 1F).

Step C. (S)-4-Amino-N-(2-chloro-4-methylpyridin-3-yl)-5-fluoro-2-((1,1,1-trifluoropropan-2-yl)oxy)benzamide. A mixture of (S)-N-(2-chloro-4-methylpyridin-3-yl)-4-((diphenylmethylene)amino)-5-fluoro-2-((1,1,1-trifluoropropan-2-yl)oxy)benzamide (400 mg, 719 μmol) and HCl (4 M dioxane solution, 0.6 mL, 2.4 mmol) in MeOH (5 mL) was stirred at room temperature for 3 hours. The mixture reaction was concentrated and the pH was adjusted to 8 by sat. aq NaHCO₃. The mixture was extracted by ethyl acetate (20 mL). The organic layer was washed by brine (15 mL), dried by Na₂SO₄, filtered and concentrated. The residue was purified by column chromatography (SiO₂, gradient elution: 0-100% ethyl acetate in petroleum ether) to give the title compound as a yellow oil. MS (ESI): mass calcd. for C₁₆H₁₄ClF₄N₃O₂, 391.1; m/z found, 392.1 [M+H]⁺. ¹H NMR (400 MHz, CDCl₃) δ=9.06 (s, 1H), 8.14-8.20 (m, 1H), 7.92 (d, J=12.0 Hz, 1H), 7.17 (d, J=5.0 Hz, 1H), 6.37 (d, J=6.8 Hz, 1H), 4.85 (dt, J=12.4, 1H), 4.24 (s, 2H), 2.33 (s, 3H), 1.64 (d, J=6.8 Hz, 3H); ¹⁹F NMR (376 MHz, CDCl₃) δ=−78.18 (s, 1F), −141.84 (s, 1F).

Step D. (S)-Phenyl (4-((2-chloro-4-methylpyridin-3-yl)carbamoyl)-2-fluoro-5-((1,1,1-trifluoropropan-2-yl)oxy)phenyl)carbamate. To a solution consisting of (S)-4-amino-N-(2-chloro-4-methylpyridin-3-yl)-5-fluoro-2-((1,1,1-trifluoropropan-2-yl)oxy)benzamide (250 mg, 395 ummol, 62% purity) and phenyl carbonochloridate (74 mg, 473 μmol) in THE (5 mL) was added pyridine (63 mg, 791 μmol) at 0° C. The mixture was stirred at 40° C. for 2 hours. The reaction mixture was concentrated. The residue was purified by column chromatography (SiO₂, gradient elution: 0-100% ethyl acetate in petroleum ether) to give the title compound as a yellow oil. MS (ESI): mass calcd. for C₂₃H₁₈ClF₄N₃O₄, 511.1; m/z found, 512.1 [M+H]⁺.

Step E. (R)-2-(((tert-Butyldiphenylsilyl)oxy)methyl)-N-(4-((2-chloro-4-methylpyridin-3-yl)carbamoyl)-2-fluoro-5-(((S)-1,1,1-trifluoropropan-2-yl)oxy)phenyl)pyrrolidine-1-carboxamide. The mixture of (S)-phenyl (4-((2-chloro-4-methylpyridin-3-yl)carbamoyl)-2-fluoro-5-((1,1,1-trifluoropropan-2-yl)oxy)phenyl)carbamate (80 mg, 111 μmol, 71% purity) and (R)-2-(((tert-butyldiphenylsilyl)oxy)methyl)pyrrolidine (Intermediate 3, 57 mg, 166.9 μmol) in THE (3 mL) was added pyridine (26 mg, 334 μmol) and the mixture reaction was stirred at 70° C. overnight then cooled down to room temperature. The mixture was quenched with sat. aq. NH₄Cl (10 mL) and the mixture was extracted with ethyl acetate (10 mL×2). The combined organic layers were dried over Na₂SO₄, filtered and concentrated in vacuum. The residue was purified by column chromatography (SiO₂, gradient eluent: petroleum ether/ethyl acetate=100/0 to 15/85) to give the title compound as a yellow oil. MS (ESI): mass calcd. for C₃₈H₄₁ClF₄N₄O₄Si, 756.3; m/z found, 757.3 [M+H]⁺.

Step F. (R)-N-(4-((2-Chloro-4-methylpyridin-3-yl)carbamoyl)-2-fluoro-5-(((S)-1,1,1-trifluoropropan-2-yl)oxy)phenyl)-2-(hydroxymethyl)pyrrolidine-1-carboxamide. (R)-2-(((tert-butyldiphenylsilyl)oxy)methyl)-N-(4-((2-chloro-4-methylpyridin-3-yl)carbamoyl)-2-fluoro-5-(((S)-1,1,1-trifluoropropan-2-yl)oxy)phenyl)pyrrolidine-1-carboxamide (80 mg, 81 μmol) in 4 M HCl solution in methanol (5 mL) was stirred at room temperature for 2 hours. The mixture was evaporated under vacuum. The residue was purified by preparative reversed phase HPLC (Stationary phase: Boston Prime C18, 5 μm, 150×30 mm; Mobile phase: water (0.04% NH₃H₂O+10 mM NH₄HCO₃) (A)-MeCN (B), gradient elution: 50-80% B in A over 7 min, flow rate: 25 mL/min) to give the title compound as an off-white powder. MS (ESI): mass calcd. for C₂₂H₂₃ClF₄N₄O₄, 518.1; m/z found, 519.1 [M+H]⁺. ¹H NMR (400 MHz, CDCl₃) δ=9.21 (s, 1H), 8.23 (dd, J=5.6, 16.7 Hz, 2H), 7.98 (d, J=12.0 Hz, 1H), 7.19 (d, J=4.8 Hz, 1H), 5.08-4.99 (m, 1H), 4.17 (br s, 1H), 3.89-3.79 (m, 1H), 3.76-3.66 (m, 2H), 3.52 (br s, 1H), 2.35 (s, 3H), 2.14 (qd, J=8.4, 12.6 Hz, 1H), 1.99 (br s, 2H), 1.74 (br s, 1H), 1.68 (d, J=6.3 Hz, 3H); ¹⁹F NMR (376 MHz, CDCl₃) δ=−78.25 (s, 1F), −135.77-−139.68 (m, 1F).

### Example 37: (R)-N-(4-((5-Chloro-3-methyl-1H-pyrazol-4-yl)carbamoyl)-2-fluoro-5-(((S)-1,1,1-trifluoropropan-2-yl)oxy)phenyl)-2-(hydroxymethyl)pyrrolidine-1-carboxamide

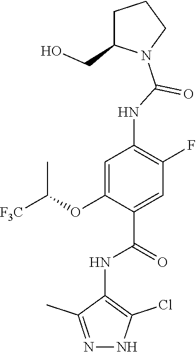 [SMILES: Cc1n[nH]c(Cl)c1NC(=O)c1cc(F)c(NC(=O)N2CCC[C@@H]2CO)cc1O[C@@H](C)C(F)(F)F]

Step A. Methyl 4-((R)-2-(((tert-butyldiphenylsilyl)oxy)methyl)pyrrolidine-1-carboxamido)-5-fluoro-2-(((S)-1,1,1-trifluoropropan-2-yl)oxy)benzoate. To a mixture of (S)-methyl 4-amino-5-fluoro-2-((1,1,1-trifluoropropan-2-yl)oxy)benzoate (Intermediate 7, 300 mg, 1.1 mmol) in pyridine (3 mL) was added phenyl chloroformate (198 mg, 1.3 mmol). The resulting mixture was stirred at 40° C. overnight. The mixture was cooled to room temperature. (R)-2-(((tert-butyldiphenylsilyl)oxy)methyl)pyrrolidine (Intermediate 3, 470 mg, 1.4 mmol) was dissolved in THE (3 mL), then added to the above reaction mixture. The resulting reaction mixture was stirred at 80° C. for 16 hours, then cooled to room temperature. The mixture was concentrated under vacuum. The crude was purified by flash column chromatography over silica gel (gradient elution: 0-16% ethyl acetate in petroleum ether) to give the title compound as a light yellow oil. MS (ESI): mass calcd. for C₃₃H₃₈F₄N₂O₅Si, 646.25; m/z found, 647.3 [M+H]⁺. ¹H NMR (400 MHz, CDCl₃) δ=8.18 (d, J=6.4 Hz, 1H), 7.62 (br dd, J=4.1, 6.3 Hz, 4H), 7.52 (br d, J=11.7 Hz, 1H), 7.43-7.28 (m, 6H), 4.70 (td, J=6.3, 12.4 Hz, 1H), 4.14 (br d, J=11.0 Hz, 1H), 3.84 (s, 3H), 3.73 (br d, J=6.4 Hz, 1H), 3.67-3.54 (m, 2H), 3.43-3.28 (m, 1H), 2.02-1.80 (m, 3H), 1.73 (br s, 1H), 1.52 (d, J=6.4 Hz, 3H), 1.00 (s, 9H); ¹⁹F NMR (376 MHz, CDCl₃) δ=−78.61 (br d, J=5.9 Hz, 1F).

Step B. 5-Fluoro-4-((R)-2-(hydroxymethyl)pyrrolidine-1-carboxamido)-2-(((S)-1,1,1-trifluoropropan-2-yl)oxy)benzoic acid. LiOH.H₂O (57 mg, 1.36 mmol) was slowly added to the mixture of methyl 4-((R)-2-(((tert-butyldiphenylsilyl)oxy)methyl)pyrrolidine-1-carboxamido)-5-fluoro-2-(((S)-1,1,1-trifluoropropan-2-yl)oxy)benzoate (400 mg, 454 μmol) in THE and H₂O (v/v, 3/1, 3 mL) at room temperature. The mixture was stirred at 40° C. overnight, then cooled to room temperature. The mixture was diluted with ethyl acetate (30 mL) and H₂O (15 mL), adjusted the pH-6 with 1 M HCl solution and separated. The aqueous phase was extracted with ethyl acetate (30 mL). The organic layers were combined, washed by brine (15 mL) twice, dried over Na₂SO₄, filtered and concentrated to give the residue. The residue was purified by column chromatography (SiO₂, gradient elution: 0-15% MeOH in CH₂Cl₂) to give the title compound as a white solid. MS (ESI): mass calcd. for C₁₆H₁₈F₄N₂O₅, 394.12; m/z found, 395.1 [M+H]⁺. ¹H NMR (400 MHz, CDCl₃) δ=9.58 (br s, 1H), 8.18 (d, J=6.4 Hz, 1H), 7.77 (d, J=11.5 Hz, 1H), 4.94 (td, J=6.1, 12.3 Hz, 1H), 4.10 (br s, 1H), 3.80 (d, J=10.6 Hz, 1H), 3.76-3.61 (m, 2H), 3.45 (s, 1H), 2.17-2.02 (m, 1H), 1.94 (s, 2H), 1.68 (s, 2H), 1.60 (d, J=6.4 Hz, 3H); ¹⁹F NMR (376 MHz, CDCl₃) δ=−78.60 (d, J=5.9 Hz, 1F).

Step C. (R)-N-(4-((5-Chloro-3-methyl-1H-pyrazol-4-yl)carbamoyl)-2-fluoro-5-(((S)-1,1,1-trifluoropropan-2-yl)oxy)phenyl)-2-(hydroxymethyl)pyrrolidine-1-carboxamide. To the mixture of 5-fluoro-4-((R)-2-(hydroxymethyl)pyrrolidine-1-carboxamido)-2-(((S)-1,1,1-trifluoropropan-2-yl)oxy)benzoic acid (100 mg, 254 μmol) and 5-chloro-3-methyl-1H-pyrazol-4-amine (116 mg, 888 μmol) in CH₂Cl₂ (1 mL) was added Et₃N (0.17 mL, 1.27 mmol). The mixture was stirred at room temperature for 10 min, then T3P (50% solution in EtOAc, 350 mg, 761 μmol) was added.

The resulting reaction mixture was stirred at 50° C. for 16 hours, then cooled to room temperature. The mixture was washed with saturated aqueous NaHCO₃ solution (1 mL), extracted with ethyl acetate (3 mL×3), concentrated under vacuum to give the residue. The residue was purified by preparative high-performance liquid chromatography (Stationary phase: Boston Prime C18, 5 μm, 150×30 mm; Mobile phase: water (0.04% NH₃H₂O+10 mM NH₄HCO₃) (A)-MeCN (B), gradient elution: 35-65% B in A over 7 min, flow rate: 25 mL/min) to give the title compound as an off-white powder. MS (ESI): mass calcd. for C₂₀H₂₂ClF₄N₅O₄, 507.13; m/z found, 508.0 [M+H]⁺. ¹H NMR (400 MHz, CDCl₃) δ=10.45 (br s, 1H), 8.78 (s, 1H), 8.17 (d, J=6.3 Hz, 1H), 7.89 (d, J=12.0 Hz, 1H), 5.03-4.90 (m, 1H), 4.12 (s, 1H), 3.82-3.62 (m, 3H), 3.47 (s, 1H), 2.26 (s, 3H), 2.16-2.04 (m, 1H), 1.95 (br s, 2H), 1.74-1.69 (m, 1H), 1.63 (s, 3H); ¹⁹F NMR (376 MHz, CDCl₃) δ=−78.38 (s, 1F), −137.28 (s, 1F).

### Example 38: (R)-N-(2-Fluoro-4-((2-methoxy-4-methylpyridin-3-yl)carbamoyl)-5-(((S)-1,1,1-trifluoropropan-2-yl)oxy)phenyl)-2-(hydroxymethyl)pyrrolidine-1-carboxamide

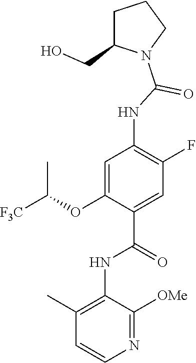 [SMILES: COc1nccc(C)c1NC(=O)c1cc(F)c(NC(=O)N2CCC[C@@H]2CO)cc1O[C@@H](C)C(F)(F)F]

Step A. (R)-2-(((tert-Butyldiphenylsilyl)oxy)methyl)-N-(2-fluoro-4-((2-methoxy-4-methylpyridin-3-yl)carbamoyl)-5-(((S)-1,1,1-trifluoropropan-2-yl)oxy)phenyl)pyrrolidine-1-carboxamide. A solution of 2-methoxy-4-methylpyridin-3-amine (54 mg, 390 μmol) and AlMe₃ (2 M solution in toluene, 0.39 mL, 0.78 mmol) in DCE (3 mL) was stirred at room temperature for 0.5 hour. The above solution (1 mL) was slowly added into another vial of methyl 4-((R)-2-(((tert-butyldiphenylsilyl)oxy)methyl)pyrrolidine-1-carboxamido)-5-fluoro-2-(((S)-1,1,1-trifluoropropan-2-yl)oxy)benzoate (Example 37, Step A, 90 mg, 130 μmol) in DCE (2 mL) at room temperature, and after half an hour interval, another batch of the above solution (1 mL) was added. A total of 3 mL solution was added. The reaction mixture was stirred at room temperature overnight. 1 M aq. HCl (0.6 mL) was added, and stirred for 2 minutes. The mixture was poured into water (20 mL) and extracted with DCM (25 mL×3). The combined organic phase was washed with brine (20 mL×2), dried with anhydrous Na₂SO₄, filtered and concentrated in vacuum. The residue was purified by column chromatography (SiO₂, gradient eluent: petroleum ether/ethyl acetate=100/0 to 20/80) to give the title compound as a yellow oil. MS (ESI): mass calcd. for C39H₄₄F₄N₄O₅Si, 752.3; m/z found, 753.2 [M+H]⁺.

Step B. (R)-N-(2-Fluoro-4-((2-methoxy-4-methylpyridin-3-yl)carbamoyl)-5-(((S)-1,1,1-trifluoropropan-2-yl)oxy)phenyl)-2-(hydroxymethyl)pyrrolidine-1-carboxamide. A mixture of (R)-2-(((tert-Butyldiphenylsilyl)oxy)methyl)-N-(2-fluoro-4-((2-methoxy-4-methylpyridin-3-yl)carbamoyl)-5-(((S)-1,1,1-trifluoropropan-2-yl)oxy)phenyl)pyrrolidine-1-carboxamide (90 mg, 111 μmol) in 4 M HCl solution in methanol (5 mL) was stirred at room temperature for 2 hours. The mixture was evaporated under vacuum. The residue was purified by preparative reversed phase HPLC (Stationary phase: Boston Prime C18, 5 μm, 150×30 mm; Mobile phase: water (0.04% NH₃H₂O+10 mM NH₄HCO₃) (A)-MeCN (B), gradient elution: 45-75% B in A over 7 min, flow rate: 25 mL/min) to give the title compound as an off-white powder. MS (ESI): mass calcd. for C₂₃H₂₆F₄N₄O₅, 514.2; m/z found, 515.1 [M+H]⁺. ¹H NMR (400 MHz, CDCl₃) δ=9.03 (s, 1H), 8.17 (d, J=6.4 Hz, 1H), 7.87-7.96 (m, 2H), 6.79 (d, J=5.1 Hz, 1H), 4.97 (dt, J=12.2, 6.2 Hz, 1H), 4.13 (br s, 1H), 3.92 (s, 3H), 3.76 (br s, 1H), 3.61-3.71 (m, 2H), 3.47 (br s, 1H), 2.24 (s, 3H), 2.04-2.13 (m, 1H), 1.95 (br s, 2H), 1.68 (br s, 2H), 1.62 (br d, J=6.4 Hz, 4H); ¹⁹F NMR (376 MHz, CDCl₃) δ=ppm −78.52 (br d, J=5.9 Hz, 1F), −137.87 (br s, 1F).

### Example 39: (R)-N-(4-((3-Chloro-2-methoxy-5-methylpyridin-4-yl)carbamoyl)-2-fluoro-5-(((S)-1,1,1-trifluoropropan-2-yl)oxy)phenyl)-2-(hydroxymethyl)pyrrolidine-1-carboxamide

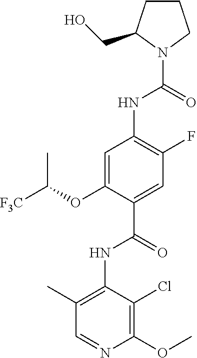 [SMILES: COc1ncc(C)c(NC(=O)c2cc(F)c(NC(=O)N3CCC[C@@H]3CO)cc2O[C@@H](C)C(F)(F)F)c1Cl]

Step A. (R)-2-(((tert-Butyldiphenylsilyl)oxy)methyl)-N-(4-((3-chloro-2-methoxy-5-methylpyridin-4-yl)carbamoyl)-2-fluoro-5-(((S)-1,1,1-trifluoropropan-2-yl)oxy)phenyl)pyrrolidine-1-carboxamide. The mixture of (S)-phenyl (4-((3-chloro-2-methoxy-5-methylpyridin-4-yl)carbamoyl)-2-fluoro-5-((1,1,1-trifluoropropan-2-yl)oxy)phenyl)carbamate (Intermediate 4, 180 mg, 321 μmol, 97% purity) and (R)-2-(((tert-butyldiphenylsilyl)oxy)methyl)pyrrolidine (Intermediate 3, 131 mg, 386 μmol) in THE (5 mL) was added pyridine (76 mg, 961 μmol) and the mixture reaction was stirred at 70° C. overnight then cooled down to room temperature. The mixture was quenched with sat. aq. NH₄Cl (10 mL) and the mixture was extracted with ethyl acetate (10 mL×2). The combined organic layers were dried over Na₂SO₄, filtered and concentrated in vacuum to give the title compound as a yellow oil. MS (ESI): mass calcd. for C₃₉H₄₃ClF₄N₄O₅Si, 786.3; m/z found, 787.3 [M+H]*.

Step B. (R)-N-(4-((3-Chloro-2-methoxy-5-methylpyridin-4-yl)carbamoyl)-2-fluoro-5-(((S)-1,1,1-trifluoropropan-2-yl)oxy)phenyl)-2-(hydroxymethyl)pyrrolidine-1-carboxamide. (R)-2-(((tert-butyldiphenylsilyl)oxy)methyl)-N-(4-((3-chloro-2-methoxy-5-methylpyridin-4-yl)carbamoyl)-2-fluoro-5-(((S)-1,1,1-trifluoropropan-2-yl)oxy)phenyl)pyrrolidine-1-carboxamide (100 mg crude, 93 μmol, 73% purity) in 4 M HCl solution in methanol (5 mL) was stirred at room temperature for 2 hours. The mixture was evaporated under vacuum. The residue was purified by preparative reversed phase HPLC (Stationary phase: Boston Prime C18, 5 μm, 150×30 mm; Mobile phase: water (0.04% NH₃H₂O+10 mM NH₄HCO₃) (A)-MeCN (B), gradient elution: 60-90% B in A over 7 min, flow rate: 25 mL/min) to give the title compound as an off-white powder. MS (ESI): mass calcd. for C₂₃H₂₅ClF₄N₄O₅, 548.1; m/z found, 549.1 [M+H]⁺. ¹HNMR (400 MHz, CDCl₃) δ=9.23 (s, 1H), 8.21 (d, J=6.4 Hz, 1H), 8.00-7.88 (m, 2H), 4.99 (td, J=6.1, 12.3 Hz, 1H), 4.12 (br s, 1H), 4.00 (s, 3H), 3.77 (br s, 1H), 3.72-3.63 (m, 2H), 3.47 (br s, 1H), 2.18 (s, 3H), 2.09 (td, J=8.4, 12.6 Hz, 1H), 1.95 (br s, 2H), 1.70 (br s, 1H), 1.62 (d, J=6.4 Hz, 3H); ¹⁹F NMR (376 MHz, CDCl₃) δ=−78.27 (br d, J=5.9 Hz, 1F), −137.93 (br s, 1F).

### Example 40: (R)-N-(2-Fluoro-4-((2-methoxy-3,5-dimethylpyridin-4-yl)carbamoyl)-5-(((S)-1,1,1-trifluoropropan-2-yl)oxy)phenyl)-2-(hydroxymethyl)pyrrolidine-1-carboxamide

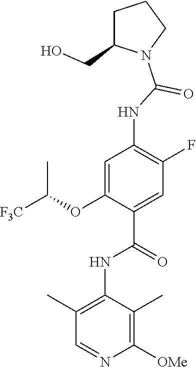 [SMILES: COc1ncc(C)c(NC(=O)c2cc(F)c(NC(=O)N3CCC[C@@H]3CO)cc2O[C@@H](C)C(F)(F)F)c1C]

Step A. (S)-Methyl 4-((tert-butoxycarbonyl)amino)-5-fluoro-2-((1,1,1-trifluoropropan-2-yl)oxy)benzoate. To the mixture of (S)-methyl 4-amino-5-fluoro-2-((1,1,1-trifluoropropan-2-yl)oxy)benzoate (Intermediate 7, 1 g, 3.4 mmol), Boc₂O (888 mg, 4.1 mmol) and triethylamine (686 mg, 6.8 mmol) in DCM (15 mL) was added DMAP (83 mg, 679 μmol) and the mixture reaction was stirred at 40° C. overnight then cooled down to room temperature. The mixture was quenched with water (20 mL) and the mixture was extracted with DCM (20 mL×2). The combined organic layers were dried over Na₂SO₄, filtered and concentrated in vacuum. The residue was purified by column chromatography (SiO₂, gradient eluent: petroleum ether/ethyl acetate=100/0 to 15/85) to provide the title compound as a yellow oil. MS (ESI): mass calcd. for C₁₆H₁₉F₄NO₅, 381.1; m/z found, 382.1 [M+H]⁺.

Step B. (S)-4-((tert-Butoxycarbonyl)amino)-5-fluoro-2-((1,1,1-trifluoropropan-2-yl)oxy)benzoic acid. LiOH.H₂O (113 mg, 2.7 mmol) was slowly added into the solution of (S)-methyl 4-((tert-butoxycarbonyl)amino)-5-fluoro-2-((1,1,1-trifluoropropan-2-yl)oxy)benzoate (480 mg, 1.13 mmol) in THE (10 mL) at room temperature. The reaction was heated to 40° C. overnight then cooled down to room temperature. The mixture was diluted by ethyl acetate (10 mL) and H₂O (5 mL), adjusted the pH to 6 by 1 M aq. HCl and separated. The aqueous layer was extracted by ethyl acetate (20 mL). The combined organic layer was washed by brine (15 mL×2), dried by Na₂SO₄, filtered and concentrated to give the title compound as a yellow oil. MS (ESI): mass calcd. for C₁₅H₁₇F4NO₅, 367.1; m/z found, 368.1 [M+H]*.

Step C. (S)-tert-Butyl (2-fluoro-4-((2-methoxy-3,5-dimethylpyridin-4-yl)carbamoyl)-5-((1,1,1-trifluoropropan-2-yl)oxy)phenyl)carbamate. POCl₃ (793 mg, 5.17 mmol) was added to a mixture of (S)-4-((tert-butoxycarbonyl)amino)-5-fluoro-2-((1,1,1-trifluoropropan-2-yl)oxy)benzoic acid (380 mg, 1.0 mmol), 2-methoxy-3,5-dimethylpyridin-4-amine (157 mg, 1.0 mmol), pyridine (1.23 g, 15.6 mmol) in DCM (10 mL) at room temperature. The reaction mixture was stirred at room temperature for 2 hours. The reaction mixture was quenched with sat. aq. K₂CO₃ (10 mL) and extracted with DCM (20 mL×3). The combined organic layers were dried over Na₂SO₄, filtered and concentrated in vacuum. The residue was purified by column chromatography (SiO₂, gradient eluent: petroleum ether/ethyl acetate=100/0 to 15/85) to give the title compound as a yellow oil. MS (ESI): mass calcd. for C₂₃H₂₇F4N₃O₅, 501.2; m/z found, 502.2 [M+H]⁺.

Step D. (S)-4-Amino-5-fluoro-N-(2-methoxy-3,5-dimethylpyridin-4-yl)-2-((1,1,1-trifluoropropan-2-yl)oxy)benzamide. A mixture of (S)-tert-butyl (2-fluoro-4-((2-methoxy-3,5-dimethylpyridin-4-yl)carbamoyl)-5-((1,1,1-trifluoropropan-2-yl)oxy)phenyl)carbamate (120 mg, 163 μmol) and 4 M HCl solution in dioxane (0.6 mL, 2.4 mmol) in MeOH (5 mL) was stirred at room temperature for 2 hours. The mixture reaction was concentrated and the pH was adjusted to 8 by sat. aq. NaHCO₃. The mixture was extracted by ethyl acetate (20 mL). The organic layer was washed by brine (15 mL), dried by Na₂SO₄, filtered and concentrated to give the title compound as a yellow oil. MS (ESI): mass calcd. for C₁₈H₁₉F₄N₃O₃, 401.1; m/z found, 402.2 [M+H]⁺.

Step E. (S)-Phenyl (2-fluoro-4-((2-methoxy-3,5-dimethylpyridin-4-yl)carbamoyl)-5-((1,1,1-trifluoropropan-2-yl)oxy)phenyl)carbamate. To a solution consisting of (S)-4-amino-5-fluoro-N-(2-methoxy-3,5-dimethylpyridin-4-yl)-2-((1,1,1-trifluoropropan-2-yl)oxy)benzamide (70 mg, 142.5 μmol) and phenyl carbonochloridate (22 mg, 142.5 μmol) in THE (5 mL) was added pyridine (23 mg, 291 μmol) at 0° C. The mixture was stirred at 40° C. for 2 hours. The mixture reaction was concentrated to give the title compound as a yellow oil. MS (ESI): mass calcd. for C₂₅H₂₃F₄N₃O₅, 521.2; m/z found, 522.2 [M+H]⁺.

Step F. (R)-2-(((tert-Butyldiphenylsilyl)oxy)methyl)-N-(2-fluoro-4-((2-methoxy-3,5-dimethylpyridin-4-yl)carbamoyl)-5-(((S)-1,1,1-trifluoropropan-2-yl)oxy)phenyl)pyrrolidine-1-carboxamide. To the mixture of (S)-phenyl (2-fluoro-4-((2-methoxy-3,5-dimethylpyridin-4-yl) carbamoyl)-5-((1,1,1-trifluoropropan-2-yl)oxy)phenyl)carbamate (80 mg crude, 84.2 μmol, 55% purity) and (R)-2-(((tert-butyldiphenylsilyl)oxy)methyl)pyrrolidine (Intermediate 3, 34 mg, 100.13 μmol) in THE (5 mL) was added pyridine (20 mg, 253 μmol), and the mixture reaction was stirred at 70° C. overnight then cooled down to room temperature. The mixture was quenched with sat. aq. NH₄Cl (10 mL) and the mixture was extracted with ethyl acetate (10 mL×2). The combined organic layers were dried over Na₂SO₄, filtered and concentrated in vacuum. The residue was purified by column chromatography (SiO₂, gradient eluent: petroleum ether/ethyl acetate=100/0 to 15/85) to give the title compound as a yellow oil. MS (ESI): mass calcd. for C₄₀H₄₆F₄N₄O₅Si, 766.3; m/z found, 767.4 [M+H]⁺.

Step G. (R)-N-(2-Fluoro-4-((2-methoxy-3,5-dimethylpyridin-4-yl)carbamoyl)-5-(((S)-1,1,1-trifluoropropan-2-yl)oxy)phenyl)-2-(hydroxymethyl)pyrrolidine-1-carboxamide. (R)-2-(((tert-butyldiphenylsilyl)oxy)methyl)-N-(2-fluoro-4-((2-methoxy-3,5-dimethylpyridin-4-yl)carbamoyl)-5-(((S)-1,1,1-trifluoropropan-2-yl)oxy)phenyl)pyrrolidine-1-carboxamide (70 mg, 81 μmol) in 4 M HCl solution in methanol (5 mL) was stirred at room temperature for 2 hours. The mixture was evaporated under vacuum. The residue was purified by preparative reversed phase HPLC (Stationary phase: Boston Prime C18, 5 μm, 150×30 mm; Mobile phase: water (0.04% NH₃H₂O+10 mM NH₄HCO₃) (A)-MeCN (B), gradient elution: 50-80% B in A over 7 min, flow rate: 25 mL/min) to give the title compound as an off-white powder. MS (ESI): mass calcd. for C₂₄H₂₈F₄N₄O₅, 528.2; m/z found, 529.1 [M+H]⁺. ¹H NMR (500 MHz, CDCl₃) δ=8.95 (s, 1H), 8.21 (d, J=6.3 Hz, 1H), 7.96 (br d, J=12.1 Hz, 1H), 7.89 (s, 1H), 5.00 (td, J=6.2, 12.5 Hz, 1H), 4.16-4.08 (m, 1H), 3.95 (s, 3H), 3.85-3.61 (m, 3H), 3.49 (br s, 1H), 2.14 (s, 3H), 2.09 (s, 3H), 1.96 (br s, 2H), 1.70 (br s, 2H), 1.63 (d, J=6.6 Hz, 3H); ¹⁹F NMR (471 MHz, CDCl₃) δ=−71.26-−83.82 (m, 1F), −141.72 (s, 1F).

### Example 41: (S)-N-(2-Chloro-4-methylpyridin-3-yl)-4-(3-(2,2-difluoroethyl)-3-ethylureido)-5-fluoro-2-((1,1,1-trifluoropropan-2-yl)oxy)benzamide

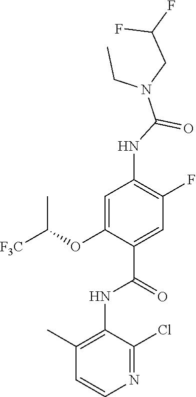 [SMILES: CCN(CC(F)F)C(=O)Nc1cc(O[C@@H](C)C(F)(F)F)c(C(=O)Nc2c(C)ccnc2Cl)cc1F]

POCl₃ (63 mg, 0.4 mmol) was added to a mixture of (S)-4-(3-(2,2-difluoroethyl)-3-ethylureido)-5-fluoro-2-((1,1,1-trifluoropropan-2-yl)oxy)benzoic acid (Intermediate 8, 90 mg, 0.2 mmol), 2-chloro-4-methylpyridin-3-amine (29 mg, 0.2 mmol), pyridine (81 mg, 1.0 mmol) in DCM (3 mL) at room temperature. The reaction mixture was stirred at room temperature for 2 hours. The reaction mixture was quenched with saturated aqueous K₂CO₃ solution and extracted with DCM. The organic layer was separated and concentrated under vacuum. The residue was purified by preparative reversed phase HPLC (Stationary phase: Phenomenex Gemini-NX C18, 3 μm, 75×30 mm; Mobile phase: water (0.04% NH₃H₂O+10 mM NH₄HCO₃) (A)-MeCN (B), gradient elution: 41-71% B in A over 8 min, flow rate: 25 mL/min). The pure fraction was lyophilized to give the title compound as an off-white powder. ¹H NMR (400 MHz, METHANOL-d₄) δ ppm 8.23 (d, J=5.0 Hz, 1H), 7.75 (d, J=6.0 Hz, 1H), 7.69 (d, J=11.0 Hz, 1H), 7.40 (d, J=5.0 Hz, 1H), 5.93-6.26 (m, 1H), 5.25 (quin, J=6.4 Hz, 1H), 3.81 (td, J=14.6, 4.1 Hz, 2H), 3.59 (q, J=7.0 Hz, 2H), 2.40 (s, 3H), 1.63 (d, J=6.3 Hz, 3H), 1.29 (t, J=7.2 Hz, 3H); ¹⁹F NMR (376 MHz, METHANOL-d₄) δ ppm −79.68 (s, 1F), −123.33 (s, 1F), −135.42 (br s, 1F); ESI-MS: m/z 527.2 [M+H]⁺;

### Example 42: (S)-N-(5-Chloro-3-methyl-1H-pyrazol-4-yl)-4-(3-(2,2-difluoroethyl)-3-ethylureido)-5-fluoro-2-((1,1,1-trifluoropropan-2-yl)oxy)benzamide

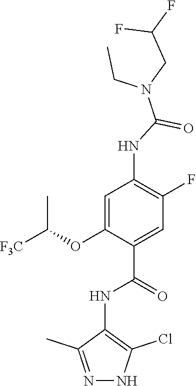 [SMILES: CCN(CC(F)F)C(=O)Nc1cc(O[C@@H](C)C(F)(F)F)c(C(=O)Nc2c(C)n[nH]c2Cl)cc1F]

To the mixture of(S)-4-(3-(2,2-difluoroethyl)-3-ethylureido)-5-fluoro-2-((1,1,1-trifluoropropan-2-yl)oxy)benzoic acid (Intermediate 8, 100 mg, 0.22 mmol) and 5-chloro-3-methyl-1H-pyrazol-4-amine (102 mg, 0.78 mmol) in DCM (5 mL) was added Et₃N (112 mg, 1.1 mmol). The mixture was stirred at room temperature for 10 min, then T3P (50% solution in EtOAc, 212 mg, 0.67 mmol) was added. The resulting reaction mixture was heated at 50° C. and stirred for 16 hours. The mixture was washed with saturated aqueous NaHCO₃ solution and extracted with EtOAc. The combined organic layers were concentrated under vacuum. The residue was purified by preparative high-performance liquid chromatography (Stationary phase: Boston Prime C18, 5 μm, 150×30 mm; Mobile phase: water (0.04% NH₁₃H₂O+10 mM NH₄HCO₃) (A)-MeCN (B), gradient elution: 40-70% B in A over 7 min, flow rate: 25 mL/min). The pure fraction was lyophilized to give the title compound as an off-white powder. ¹H NMR (400 MHz, CHLOROFORM-d) δ ppm 10.39 (br s, 1H), 8.75 (s, 1H), 8.16 (d, J=6.3 Hz, 1H), 8.02 (d, J=12.3 Hz, 1H), 7.04 (br d, J=4.5 Hz, 1H), 5.84-6.21 (m, 1H), 4.98 (dt, J=12.3, 6.1 Hz, 1H), 3.72 (td, J=14.1, 4.1 Hz, 2H), 3.51 (q, J=7.3 Hz, 2H), 2.28 (s, 3H), 1.64 (br s, 3H), 1.34 (t, J=7.2 Hz, 3H); ¹⁹F NMR (376 MHz, CHLOROFORM-d) δ ppm −80.13-−77.36 (m, 1F), −122.89-−119.72 (m, 1F), −141.09-−138.32 (m, 1F); ESI-MS: m/z 516.0 [M+H]⁺;

### Example 43: (S)-4-(3-(2,2-difluoroethyl)-3-ethylureido)-5-fluoro-N-(2-methoxy-4-methylpyridin-3-yl)-2-((1,1,1-trifluoropropan-2-yl)oxy)benzamide

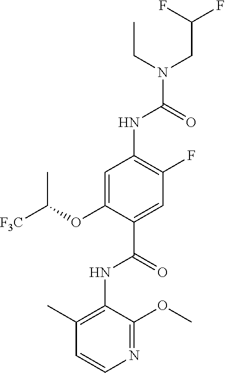 [SMILES: CCN(CC(F)F)C(=O)Nc1cc(O[C@@H](C)C(F)(F)F)c(C(=O)Nc2c(C)ccnc2OC)cc1F]

POCl₃ (109 mg, 711 μmol) was added to a mixture of (S)-4-(3-(2,2-difluoroethyl)-3-ethylureido)-5-fluoro-2-((1,1,1-trifluoropropan-2-yl)oxy)benzoic acid (Intermediate 8, 95 mg, 178 μmol), 2-methoxy-4-methylpyridin-3-amine (32 mg, 231 μmol), pyridine (84 mg, 1.1 mmol) in DCM (2 mL) at room temperature. The reaction mixture was stirred at room temperature for 1 hour. The reaction mixture was quenched with sat. aq. K₂CO₃ solution (15 mL) and extracted with DCM (12 mL). The organic layer was washed with brine (5 mL×3), dried with anhydrous Na₂SO₄, filtered and concentrated under vacuum. The residue was purified by preparative reversed phase HPLC (Stationary phase: Boston Prime C18, 5 μm, 150×30 mm; Mobile phase: water (0.04% NH₁₃H₂O+10 mM NH₄HCO₃) (A)-MeCN (B), gradient elution: 55-85% B in A over 7 min, flow rate: 25 mL/min) to give the title compound as yellow powder. MS (ESI): mass calcd. For C₂₂H₂₄F6N₄O₄, 522.2; m/z found, 523.1 [M+H]⁺. ¹H NMR (400 MHz, CDCl₃) δ=9.01 (s, 1H), 8.16 (d, J=6.3 Hz, 1H), 8.02 (d, J=12.3 Hz, 1H), 7.94 (d, J=5.3 Hz, 1H), 7.02 (br d, J=4.3 Hz, 1H), 6.82 (d, J=5.3 Hz, 1H), 5.86-6.19 (m, 1H), 4.99 (dt, J=12.4, 6.2 Hz, 1H), 3.95 (s, 3H), 3.72 (td, J=14.1, 4.1 Hz, 2H), 3.51 (q, J=7.3 Hz, 2H), 2.27 (s, 3H), 1.64 (d, J=6.3 Hz, 3H), 1.34 (t, J=7.3 Hz, 3H); ¹⁹F NMR (376 MHz, CDCl₃) δ=−78.45 (s, 1F), −121.28 (s, 1F), −140.09 (s, 1F).

### Example 44: (S)-N-(3-Chloro-2-methoxy-5-methylpyridin-4-yl)-4-(3-(2,2-difluoroethyl)-3-ethylureido)-5-fluoro-2-((1,1,1-trifluoropropan-2-yl)oxy)benzamide

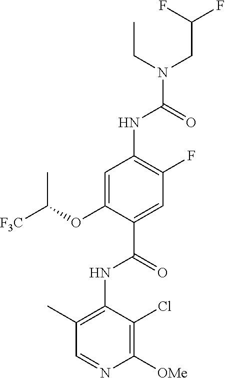 [SMILES: CCN(CC(F)F)C(=O)Nc1cc(O[C@@H](C)C(F)(F)F)c(C(=O)Nc2c(C)cnc(OC)c2Cl)cc1F]

A solution consisting of (S)-phenyl (4-((3-chloro-2-methoxy-5-methylpyridin-4-yl) carbamoyl)-2-fluoro-5-((1,1,1-trifluoropropan-2-yl)oxy)phenyl)carbamate (Intermediate 4, 120 mg crude, 214 μmol, 97% purity) and N-ethyl-2,2-difluoroethanamine (Intermediate 5, 37 mg crude as HCl salt, 257 μmol) in THE (3 mL) was added pyridine (51 mg, 642 μmol) at 70° C. for 16 hours. The mixture was evaporated under vacuum. The residue was purified by preparative high-performance liquid chromatography. The residue was purified by preparative reversed phase HPLC (Stationary phase: Boston Prime C18, 5 μm, 150×30 mm; Mobile phase: water (0.04% NH₁₃H₂O+10 mM NH₄HCO₃) (A)-MeCN (B), gradient elution: 60-90% B in A over 7 min, flow rate: 25 mL/min) to give the title compound as an off-white powder. MS (ESI): mass calcd. for C₂₂H₂₃ClF₆N₄O₄, 556.1; m/z found, 557.1 [M+H]⁺. ¹H NMR (400 MHz, CDCl₃) δ=9.20 (s, 1H), 8.17 (d, J=6.2 Hz, 1H), 8.02 (d, J=12.1 Hz, 1H), 7.93 (s, 1H), 7.02 (br d, J=4.4 Hz, 1H), 6.20-5.81 (m, 1H), 4.98 (td, J=6.0, 12.2 Hz, 1H), 4.00 (s, 3H), 3.70 (dt, J=4.2, 14.1 Hz, 2H), 3.49 (q, J=7.1 Hz, 2H), 2.18 (s, 3H), 1.63 (d, J=6.4 Hz, 3H), 1.33 (t, J=7.2 Hz, 3H); ¹⁹F NMR (376 MHz, CDCl₃) δ=−78.23 (br d, J=5.9 Hz, 1F), −121.31 (br d, J=56.5 Hz, 1F), −137.69-−142.57 (m, 1F).

### Example 45: (S)-4-(3-(2,2-Difluoroethyl)-3-ethylureido)-5-fluoro-N-(2-methoxy-3,5-dimethylpyridin-4-yl)-2-((1,1,1-trifluoropropan-2-yl)oxy)benzamide

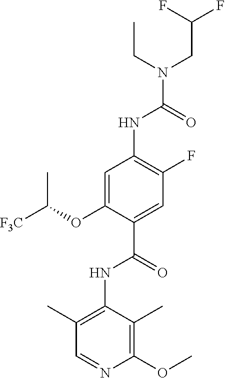 [SMILES: CCN(CC(F)F)C(=O)Nc1cc(O[C@@H](C)C(F)(F)F)c(C(=O)Nc2c(C)cnc(OC)c2C)cc1F]

POCl₃ (109 mg, 711 μmol) was added to a mixture of (S)-4-(3-(2,2-difluoroethyl)-3-ethylureido)-5-fluoro-2-((1,1,1-trifluoropropan-2-yl)oxy)benzoic acid (Intermediate 8, 90 mg, 224 μmol), 2-methoxy-3,5-dimethylpyridin-4-amine (35 mg, 230 μmol), pyridine (140 mg, 1.8 mmol) in DCM (2 mL) at room temperature. The reaction mixture was stirred at room temperature for 1 hour. The reaction mixture was quenched with sat. aq. K₂CO₃ solution (15 mL) and extracted with DCM (12 mL). The organic layer was washed with brine (5 mL×3), dried with anhydrous Na₂SO₄, filtered and concentrated under vacuum. The residue was purified by preparative reversed phase HPLC (Stationary phase: Phenomenex Gemini-NX C18, 3 μm, 75×30 mm; Mobile phase: water (0.04% NH₁₃H₂O+10 mM NH₄HCO₃) (A)-MeCN (B), gradient elution: 55-85% B in A over 6 min, flow rate: 25 mL/min) to give the title compound as a yellow powder. MS (ESI): mass calcd. for C₂₃H₂₆F₆N₄O₄, 536.2; m/z found, 537.1 [M+H]⁺. ¹H NMR (400 MHz, CDCl₃) δ=8.93 (s, 1H), 8.19 (d, J=6.2 Hz, 1H), 8.07 (d, J=12.2 Hz, 1H), 7.92 (s, 1H), 7.06 (br d, J=4.3 Hz, 1H), 5.88-6.21 (m, 1H), 5.03 (dt, J=12.4, 6.2 Hz, 1H), 3.97 (s, 3H), 3.74 (td, J=14.1, 4.2 Hz, 2H), 3.54 (q, J=7.4 Hz, 2H), 2.17 (s, 3H), 2.12 (s, 3H), 1.66 (d, J=6.4 Hz, 3H), 1.37 (t, J=7.3 Hz, 3H); ¹⁹F NMR (376 MHz, CDCl₃) δ=−78.36 (s, 1F), −121.26 (br s, 1F), −139.92 (s, 1F).

DHODH inhibitory activities of the compounds of Examples 1-45 were assessed using the following assays. The half maximal effective concentration values (EC50) are summarized in Table 3.

### Biological Assays

### In Vitro Assay: DHODH Enzymatic Assay

To detect DHODH enzyme activities, dichloroindophenol (DCIP) is added as the final electron acceptor in the assay. DCIP can accept electrons from the reduced coenzyme Q generated in the assay, or from dihydroorotate (DHO) via flavin mononucleotide (FMN) by binding presumably to the ubiquinone pocket. DCIP solutions are blue, with an intense absorbance around 600 nm, but becomes colorless upon reduction (J. Biol. Chem. (1986) 261, 11386). The assay buffer contained 50 nM HEPES, pH 7.5, 150 mM NaCl, 0.5 mM EDTA, and 0.1% Triton X-100 in MilliQ water. Substrate consisting of 20 mM DHO, 5 mM CoQ₆, and 1 mM DCIP in assay buffer, initiates the reaction. The assay is run in end-point mode by quenching the reaction with the potent DHODH inhibitor brequinar. Absorbance measurements were obtained using the BMG Phera Star plate-reading spectrophotomer. Purified human DHODH was purchased from Proteros (cat. No. PR-0044). Chemicals were purchased from Sigma-Aldrich, Teknova, and Avanti Polar Lipids. Liquid handling was performed using Labcyte Echo and Formulatrix Tempest.

### In Vitro Assay: MOLM-13 Cellular Assay

MOLM-13 cells were obtained from DSMZ and were maintained in RPMI 1640+Glutamax+25 mM HEPES (Invitrogen, catalog number 72400) supplemented with 10% heat inactivated fetal bovine serum (FBS; Invitrogen, catalog number 16140). The day prior to assay set-up, cells were pelleted, resuspended in fresh media, counted, and cells were plated at 0.4×10⁶ cell/mL in a T150 flask. On the day of the assay, cells were pelleted, resuspend in fresh media, counted and seeded at 5,000 cells/well in white opaque 96-well tissue culture treated microplates (Perkin Elmer, catalog number 6005680). Cells were exposed to different concentrations of test compounds at 37° C., 5% CO₂ for 72 hours immediately after seeding. Cell viability was acquired on a Perkin Elmer Envision 2104 multilabel reader using the CellTiter-Glo assay (Promega) according to the manufacturer's instructions.

Biological activity for selected compounds of the invention may be found in Table 3.

TABLE 3

| | DHODH Enzymatic | MOLM-13 Cellular |
|---|---|---|
| Example # | Assay EC50 (nM) | Assay EC50 (nM) |
| 1 | 1. | 0.64 |
| 2 | 20 | 12 |
| 3 | 4.1 | 1.2 |
| 4 | 7.3 | 2.1 |
| 5 | 2.8 | 0.98 |
| 6 | 3.6 | 1.2 |
| 7 | 3.5 | 1.9 |
| 8 | 100 | 53 |
| 9 | 490 | 89 |
| 10 | 4.0 | 2.0 |
| 11 | 2.4 | 0.79 |
| 12 | 5.2 | — |
| 13 | 950 | 220 |
| 14 | 7.9 | — |
| 15 | 20 | — |
| 16 | 4.7 | — |
| 17 | 200 | — |
| 18 | 2.6 | 1.5 |
| 19 | 2.3 | — |
| 20 | 2.8 | — |
| 21 | 3.8 | 1.5 |
| 22 | 7.2 | — |
| 23 | 540 | 270 |
| 24 | 1300 | 430 |
| 25 | 210 | 110 |
| 26 | 7970 | 170 |
| 27 | 2.0 | 0.96 |
| 28 | 11 | 3.6 |
| 29 | 85 | 40 |
| 30 | 120 | 57 |
| 31 | 48 | 11 |
| 32 | 13 | 5.8 |
| 33 | 9.0 | 3.2 |
| 34 | 5.4 | 1.0 |
| 35 | 57 | 25 |
| 36 | 2.6 | 6.6 |
| 37 | 0.70 | 11 |
| 38 | 0.17 | 16 |
| 39 | 2.6 | 3.5 |
| 40 | 3.6 | 25 |
| 41 | 17 | 37 |
| 42 | 3.0 | 11 |
| 43 | 24 | 30 |
| 44 | 7.0 | 9.6 |
| 45 | 27 | 63 |

While the foregoing specification teaches the principles of the present invention, with examples provided for the purpose of illustration, it will be understood that the practice of the invention encompasses all of the usual variations, adaptations and/or modifications as come within the scope of the following claims and their equivalents.

All documents cited herein are incorporated by reference.

1. A compound of Formula I 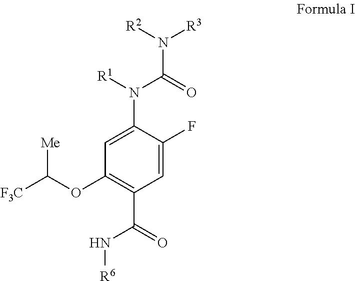
wherein:
R¹ is —H, or —C₍₁₋₄₎alkyl;
R² is —H, or —C₍₁₋₄₎alkyl, wherein said —C₍₁₋₄₎alkyl is optionally substituted with up to three fluorine atoms;
R³ is —C₍₁₋₄₎alkyl, or C₍₃₋₄₎cycloalkyl, wherein said —C₍₁₋₄₎alkyl is optionally substituted with —CN, —OCH₃, —OCF₃, —OH, or up to six fluorine atoms;
or R² and R³ may be taken together with their attached nitrogen, to form a ring selected from the group consisting of, azetidinyl, pyrrolidinyl, pyrrolidinonyl, morpholinyl, or piperidinyl, wherein said azetidinyl, pyrrolidinyl, pyrrolidinonyl, morpholinyl, or piperidinyl is optionally substituted with one or two fluorine atoms, or —OH, —OCH₃, —CF₃, —OCF₃, —OCHF₂, or —C₍₁₋₄₎alkyl, wherein said —C₍₁₋₄₎alkyl is optionally substituted with —OCH₃, —OCF₃, —OCHF₂, —OH, or up to six fluorine atoms;
R⁶ is selected from the group consisting of: 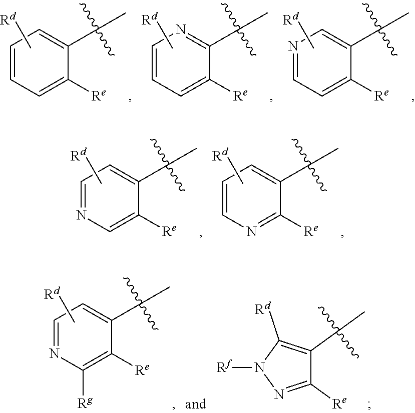
R^{d} is selected from the group consisting of: H; halo; C₁₋₆alkyl; C₁₋₆alkyl substituted with a member selected from the group consisting of: OH, OCH₃, SCH₃, and OCF₃; C₁₋₆haloalkyl; C₁₋₆haloalkyl substituted with a member selected from the group consisting of: OH, and OCH₃; and OC₁₋₆alkyl;
R^{e} is selected from the group consisting of: halo; C₁₋₆alkyl; C₁₋₆alkyl substituted with a member selected from the group consisting of: OH, OCH₃, SCH₃, and OCF₃; C₁₋₆haloalkyl; C₁₋₆haloalkyl substituted with a member selected from the group consisting of: OH, and OCH₃; and OC₁₋₆alkyl;
R^{f} is selected from the group consisting of: H; C₁₋₆alkyl; C₁₋₆alkyl substituted with a member selected from the group consisting of: OH, OCH₃, SCH₃, and OCF₃; C₁₋₆haloalkyl; and C₁₋₆haloalkyl substituted with a member selected from the group consisting of: OH, and OCH₃; and
R^{g} is selected from the group consisting of: H; C₁₋₆alkyl; C₁₋₆alkyl substituted with a member selected from the group consisting of: OH, OCH₃, SCH₃, and OCF₃; C₁₋₆haloalkyl; C₁₋₆haloalkyl substituted with a member selected from the group consisting of: OH, and OCH₃; and OC₁₋₆alkyl;
or a pharmaceutically acceptable salt, isotope, N-oxide, solvate, or stereoisomer thereof.

2. A compound of claim 1, which is a compound of Formula II 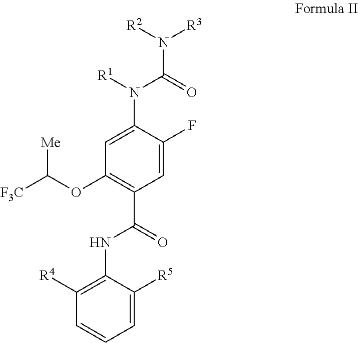
wherein:
R¹ is H, or —C₍₁₋₄₎alkyl;
R² is —H, or —C₍₁₋₄₎alkyl, wherein said —C₍₁₋₄₎alkyl is optionally substituted with up to three fluorine atoms;
R³ is —C₍₁₋₄₎alkyl, or C₍₃₋₄₎cycloalkyl, wherein said —C₍₁₋₄₎alkyl is optionally substituted with —CN, —OCH₃, —OCF₃, —OH, or up to six fluorine atoms;
or R² and R³ may be taken together with their attached nitrogen, to form a ring selected from the group consisting of, azetidinyl, pyrrolidinyl, pyrrolidinonyl, morpholinyl, or piperidinyl, wherein said azetidinyl, pyrrolidinyl, pyrrolidinonyl, morpholinyl, or piperidinyl is optionally substituted with one or two fluorine atoms, or —OH, —OCH₃, —CF₃, —OCF₃, —OCHF₂, or —C₍₁₋₄₎alkyl, wherein said —C₍₁₋₄₎alkyl is optionally substituted with —OCH₃, —OCF₃, —OCHF₂, —OH, or up to six fluorine atoms;
R⁴ and R⁵ are independently selected from —F, —Cl, —Br, and —I;
or a pharmaceutically acceptable salt, isotope, N-oxide, solvate, or stereoisomer thereof.

3. A compound of claim 2, which is a compound of Formula III 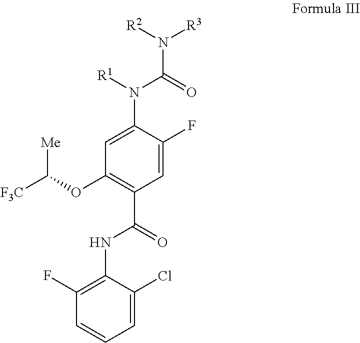
wherein:
R¹ is H, or —CH₃;
R² is —H, or —C₍₁₋₂₎alkyl, wherein said —C₍₁₋₂₎alkyl is optionally substituted with up to three fluorine atoms;
R³ is —C₍₁₋₄₎alkyl, or C₍₃₋₄₎cycloalkyl, wherein said —C₍₁₋₄₎alkyl is optionally substituted with —CN, —OCH₃, —OH, or up to six fluorine atoms;
or R² and R³ may be taken together with their attached nitrogen, to form a ring selected from the group consisting of, azetidinyl, pyrrolidinyl, pyrrolidinonyl, or piperidinyl, wherein said azetidinyl, pyrrolidinyl, pyrrolidinonyl, or piperidinyl is optionally substituted with one or two fluorine atoms, or —OH, —OCH₃, —CF₃, —OCF₃, —OCHF₂, or —C₍₁₋₄₎alkyl, wherein said —C₍₁₋₄₎alkyl is optionally substituted with —OCH₃, —OCF₃, —OCHF₂, —OH, or up to six fluorine atoms;
or a pharmaceutically acceptable salt, isotope, N-oxide, solvate, or stereoisomer thereof.

4. The compound of claim 1, wherein
R² is —H, or —C₍₁₋₂₎alkyl;
R³ is —C₍₁₋₄₎alkyl, or C₍₃₋₄₎cycloalkyl, wherein said —C₍₁₋₄₎alkyl is optionally substituted with —CN, —OCH₃, —OH, or up to three fluorine atoms;
or R² and R³ may be taken together with their attached nitrogen, to form a ring selected from the group consisting of, azetidinyl, pyrrolidinyl, pyrrolidinonyl, or piperidinyl, wherein said azetidinyl, pyrrolidinyl, pyrrolidinonyl, or piperidinyl is optionally substituted with one or two fluorine atoms, or —OH, —OCH₃, —OCF₃, —OCHF₂, or —C₍₁₋₄₎alkyl, wherein said —C₍₁₋₄₎alkyl is optionally substituted with —OCH₃, —OCF₃, —OCHF₂, —OH, or up to two fluorine atoms;
or a pharmaceutically acceptable salt, isotope, N-oxide, solvate, or stereoisomer thereof.

5. The compound of claim 4, wherein
R³ is —C₍₁₋₄₎alkyl, or C₍₃₋₄₎cycloalkyl, wherein said —C₍₁₋₄₎alkyl is optionally substituted with —CN, —OCH₃, —OH, or up to two fluorine atoms;
or R² and R³ may be taken together with their attached nitrogen, to form a ring selected from the group consisting of, azetidinyl, pyrrolidinyl, pyrrolidinonyl, or piperidinyl, wherein said azetidinyl, pyrrolidinyl, pyrrolidinonyl, or piperidinyl is optionally substituted with one or two fluorine atoms, or —OH, —OCH₃, or —C₍₁₋₄₎alkyl, wherein said —C₍₁₋₄₎alkyl is optionally substituted with —OCH₃, —OH, or up to two fluorine atoms;
or a pharmaceutically acceptable salt, isotope, N-oxide, solvate, or stereoisomer thereof.

6. The compound of claim 5, wherein
R³ is —C₍₁₋₃₎alkyl, or cyclopropyl, wherein said —C₍₁₋₃₎alkyl is optionally substituted with —CN, —OCH₃, —OH, or up to two fluorine atoms;
or R² and R³ may be taken together with their attached nitrogen, to form a ring selected from the group consisting of, azetidinyl, pyrrolidinyl, pyrrolidinonyl, or piperidinyl, wherein said azetidinyl, pyrrolidinyl, pyrrolidinonyl, or piperidinyl is optionally substituted with one or two fluorine atoms, or —OH, —OCH₃, or —C₍₁₋₃₎alkyl, wherein said —C₍₁₋₃₎alkyl is optionally substituted with —OCH₃, —OH, or up to two fluorine atoms;
or a pharmaceutically acceptable salt, isotope, N-oxide, solvate, or stereoisomer thereof.

7. A compound selected from the group consisting of: [chemical structure] 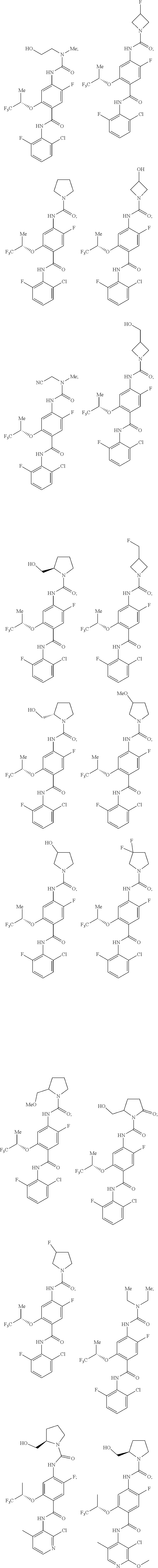 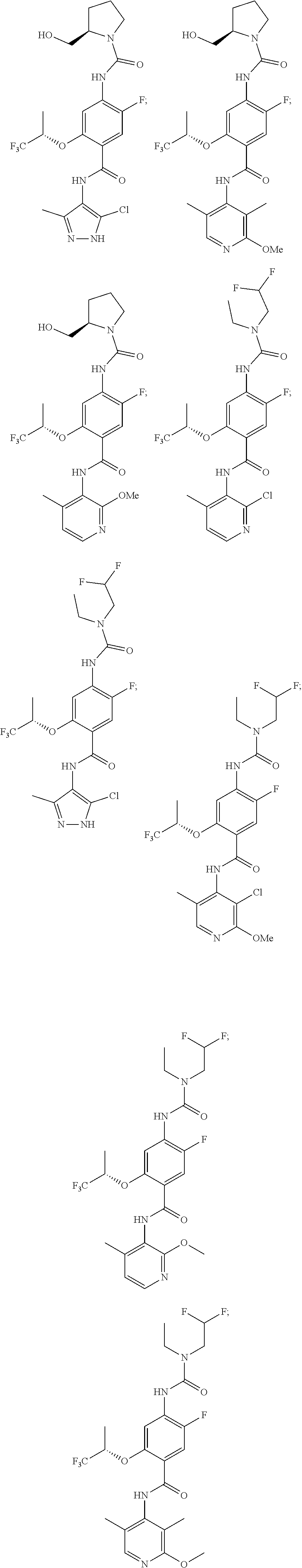
or a pharmaceutically acceptable salt, isotope, N-oxide, solvate, or stereoisomer thereof.

8. A pharmaceutical composition comprising: (A) a compound according to claim 1, or a pharmaceutically acceptable salt, solvate, stereoisomer, isotopic variant, or N-oxide thereof; and (B) at least one pharmaceutically acceptable excipient.

9. A pharmaceutical composition comprising an effective amount of:
(S)-N-(2-chloro-6-fluorophenyl)-4-(3,3-diethylureido)-5-fluoro-2-((1,1,1-trifluoropropan-2-yl)oxy)benzamide;
(S)-N-(4-((2-chloro-6-fluorophenyl)carbamoyl)-2-fluoro-5-((1,1,1-trifluoropropan-2-yl)oxy)phenyl)piperidine-1-carboxamide;
(S)-N-(2-chloro-6-fluorophenyl)-4-(3-ethyl-3-(2-hydroxyethyl)ureido)-5-fluoro-2-((1,1,1-trifluoropropan-2-yl)oxy)benzamide;
(S)-N-(2-chloro-6-fluorophenyl)-4-(3-ethyl-3-isopropylureido)-5-fluoro-2-((1,1,1-trifluoropropan-2-yl)oxy)benzamide;
(S)-N-(2-chloro-6-fluorophenyl)-4-(3-cyclopropyl-3-ethylureido)-5-fluoro-2-((1,1,1-trifluoropropan-2-yl)oxy)benzamide;
(S)-N-(2-chloro-6-fluorophenyl)-4-(3-ethyl-3-(2-methoxyethyl)ureido)-5-fluoro-2-((1,1,1-trifluoropropan-2-yl)oxy)benzamide;
(S)-N-(2-chloro-6-fluorophenyl)-4-(3-(2,2-difluoroethyl)-3-ethylureido)-5-fluoro-2-((1,1,1-trifluoropropan-2-yl)oxy)benzamide;
(S)-N-(2-chloro-6-fluorophenyl)-4-(3-ethylureido)-5-fluoro-2-((1,1,1-trifluoropropan-2-yl)oxy)benzamide;
(S)-N-(2-chloro-6-fluorophenyl)-5-fluoro-4-(3-isopropylureido)-2-((1,1,1-trifluoropropan-2-yl)oxy)benzamide;
(S)-N-(2-chloro-6-fluorophenyl)-4-(3,3-dimethylureido)-5-fluoro-2-((1,1,1-trifluoropropan-2-yl)oxy)benzamide;
(S)-N-(2-chloro-6-fluorophenyl)-4-(3-ethyl-3-methylureido)-5-fluoro-2-((1,1,1-trifluoropropan-2-yl)oxy)benzamide;
N-(4-((2-chloro-6-fluorophenyl)carbamoyl)-2-fluoro-5-(((S)-1,1,1-trifluoropropan-2-yl)oxy)phenyl)-2-(hydroxymethyl)azetidine-1-carboxamide;
N-(4-((2-chloro-6-fluorophenyl)carbamoyl)-2-fluoro-5-(((S)-1,1,1-trifluoropropan-2-yl)oxy)phenyl)-3-(hydroxymethyl)pyrrolidine-1-carboxamide;
(S)-N-(2-chloro-6-fluorophenyl)-5-fluoro-4-(3-methyl-3-propylureido)-2-((1,1,1-trifluoropropan-2-yl)oxy)benzamide;
(S)-N-(2-chloro-6-fluorophenyl)-4-(3-ethyl-3-propylureido)-5-fluoro-2-((1,1,1-trifluoropropan-2-yl)oxy)benzamide;
(S)-N-(2-chloro-6-fluorophenyl)-5-fluoro-4-(3-(2-fluoroethyl)-3-methylureido)-2-((1,1,1-trifluoropropan-2-yl)oxy)benzamide;
(S)-N-(2-chloro-6-fluorophenyl)-4-(3-(2-cyanoethyl)-3-methylureido)-5-fluoro-2-((1,1,1-trifluoropropan-2-yl)oxy)benzamide;
(S)-N-(2-chloro-6-fluorophenyl)-5-fluoro-4-(3-(2-hydroxyethyl)-3-methylureido)-2-((1,1,1-trifluoropropan-2-yl)oxy)benzamide;
(S)-N-(4-((2-chloro-6-fluorophenyl)carbamoyl)-2-fluoro-5-((1,1,1-trifluoropropan-2-yl)oxy)phenyl)pyrrolidine-1-carboxamide;
(S)-N-(4-((2-chloro-6-fluorophenyl)carbamoyl)-2-fluoro-5-((1,1,1-trifluoropropan-2-yl)oxy)phenyl)azetidine-1-carboxamide;
N-(4-((2-chloro-6-fluorophenyl)carbamoyl)-2-fluoro-5-(((S)-1,1,1-trifluoropropan-2-yl)oxy)phenyl)-2-(hydroxymethyl)pyrrolidine-1-carboxamide;
(S)-N-(4-((2-chloro-6-fluorophenyl)carbamoyl)-2-fluoro-5-((1,1,1-trifluoropropan-2-yl)oxy)phenyl)-3-fluoroazetidine-1-carboxamide;
(S)-N-(4-((2-chloro-6-fluorophenyl)carbamoyl)-2-fluoro-5-((1,1,1-trifluoropropan-2-yl)oxy)phenyl)-3-hydroxyazetidine-1-carboxamide;
(S)-N-(4-((2-chloro-6-fluorophenyl)carbamoyl)-2-fluoro-5-((1,1,1-trifluoropropan-2-yl)oxy)phenyl)-3-(hydroxymethyl)azetidine-1-carboxamide;
(S)-N-(4-((2-chloro-6-fluorophenyl)carbamoyl)-2-fluoro-5-((1,1,1-trifluoropropan-2-yl)oxy)phenyl)-3-(fluoromethyl)azetidine-1-carboxamide;
(S)-N-(2-chloro-6-fluorophenyl)-4-(3-(cyanomethyl)-3-methylureido)-5-fluoro-2-((1,1,1-trifluoropropan-2-yl)oxy)benzamide;
(R)-N-(4-((2-chloro-6-fluorophenyl)carbamoyl)-2-fluoro-5-(((S)-1,1,1-trifluoropropan-2-yl)oxy)phenyl)-2-(hydroxymethyl)pyrrolidine-1-carboxamide;
(S)-N-(4-((2-chloro-6-fluorophenyl)carbamoyl)-2-fluoro-5-(((S)-1,1,1-trifluoropropan-2-yl)oxy)phenyl)-2-(hydroxymethyl)pyrrolidine-1-carboxamide;
N-(4-((2-chloro-6-fluorophenyl)carbamoyl)-2-fluoro-5-(((S)-1,1,1-trifluoropropan-2-yl)oxy)phenyl)-3-methoxypyrrolidine-1-carboxamide;
N-(4-((2-chloro-6-fluorophenyl)carbamoyl)-2-fluoro-5-(((S)-1,1,1-trifluoropropan-2-yl)oxy)phenyl)-3-hydroxypyrrolidine-1-carboxamide;
N-(4-((2-chloro-6-fluorophenyl)carbamoyl)-2-fluoro-5-(((S)-1,1,1-trifluoropropan-2-yl)oxy)phenyl)-2-(methoxymethyl)pyrrolidine-1-carboxamide;
N-(4-((2-chloro-6-fluorophenyl)carbamoyl)-2-fluoro-5-(((S)-1,1,1-trifluoropropan-2-yl)oxy)phenyl)-3-fluoropyrrolidine-1-carboxamide;
(S)-N-(4-((2-chloro-6-fluorophenyl)carbamoyl)-2-fluoro-5-((1,1,1-trifluoropropan-2-yl)oxy)phenyl)-3,3-difluoropyrrolidine-1-carboxamide;
N-(4-((2-chloro-6-fluorophenyl)carbamoyl)-2-fluoro-5-(((S)-1,1,1-trifluoropropan-2-yl)oxy)phenyl)-2-(hydroxymethyl)-5-oxopyrrolidine-1-carboxamide;
(S)-N-(2-chloro-6-fluorophenyl)-4-(3,3-diethyl-1-methylureido)-5-fluoro-2-((1,1,1-trifluoropropan-2-yl)oxy)benzamide;
(R)-N-(4-((2-Chloro-4-methylpyridin-3-yl)carbamoyl)-2-fluoro-5-(((S)-1,1,1-trifluoropropan-2-yl)oxy)phenyl)-2-(hydroxymethyl)pyrrolidine-1-carboxamide;
(R)-N-(4-((5-Chloro-3-methyl-1H-pyrazol-4-yl)carbamoyl)-2-fluoro-5-(((S)-1,1,1-trifluoropropan-2-yl)oxy)phenyl)-2-(hydroxymethyl)pyrrolidine-1-carboxamide;
(R)-N-(2-Fluoro-4-((2-methoxy-4-methylpyridin-3-yl)carbamoyl)-5-(((S)-1,1,1-trifluoropropan-2-yl)oxy)phenyl)-2-(hydroxymethyl)pyrrolidine-1-carboxamide;
(R)-N-(4-((3-Chloro-2-methoxy-5-methylpyridin-4-yl)carbamoyl)-2-fluoro-5-(((S)-1,1,1-trifluoropropan-2-yl)oxy)phenyl)-2-(hydroxymethyl)pyrrolidine-1-carboxamide;
(R)-N-(2-Fluoro-4-((2-methoxy-3,5-dimethylpyridin-4-yl)carbamoyl)-5-(((S)-1,1,1-trifluoropropan-2-yl)oxy)phenyl)-2-(hydroxymethyl)pyrrolidine-1-carboxamide;
(S)-N-(2-Chloro-4-methylpyridin-3-yl)-4-(3-(2,2-difluoroethyl)-3-ethylureido)-5-fluoro-2-((1,1,1-trifluoropropan-2-yl)oxy)benzamide;
(S)-N-(5-Chloro-3-methyl-1H-pyrazol-4-yl)-4-(3-(2,2-difluoroethyl)-3-ethylureido)-5-fluoro-2-((1,1,1-trifluoropropan-2-yl)oxy)benzamide;
(S)-4-(3-(2,2-difluoroethyl)-3-ethylureido)-5-fluoro-N-(2-methoxy-4-methylpyridin-3-yl)-2-((1,1,1-trifluoropropan-2-yl)oxy)benzamide;
(S)-N-(3-Chloro-2-methoxy-5-methylpyridin-4-yl)-4-(3-(2,2-difluoroethyl)-3-ethylureido)-5-fluoro-2-((1,1,1-trifluoropropan-2-yl)oxy)benzamide;
(S)-4-(3-(2,2-Difluoroethyl)-3-ethylureido)-5-fluoro-N-(2-methoxy-3,5-dimethylpyridin-4-yl)-2-((1,1,1-trifluoropropan-2-yl)oxy)benzamide;
or a pharmaceutically acceptable salt, solvate, stereoisomer, isotopic variant, or N-oxide thereof;
and at least one pharmaceutically acceptable excipient.

10. A method of treating a subject suffering from or diagnosed with a disease, disorder, or medical condition comprising inhibiting or altering dihydroorotate oxygenase enzyme activity in the subject by administering to the subject an effective amount of at least one compound according to claim 1, or a pharmaceutically acceptable salt, solvate, stereoisomer, isotopic variant, or N-oxide thereof.

11. The method according to claim 10, wherein the disorder, disease or medical condition is selected from the group consisting of: inflammatory disorders and autoimmune disorders.

12. The method according to claim 10, wherein the disorder, disease or medical condition is cancer.

13. The method according to claim 10, wherein the disorder, disease or medical condition is selected from the group consisting of: lymphomas, leukemias, carcinomas, and sarcomas.

14. The method according to claim 10, wherein the disorder, disease or medical condition is selected from the group consisting of: acute lymphoblastic leukemia, acute myeloid leukemia, (acute) T-cell leukemia, acute lymphoblastic leukemia, acute lymphocytic leukemia, acute monocytic leukemia, acute promyelocytic leukemia, bisphenotypic B myelomonocytic leukemia, chronic lymphocytic leukemia, chronic myelogenous leukemia, chronic myeloid leukemia, chronic myelomonocytic leukemia, large granular lymphocytic leukemia, plasma cell leukemia, and also myelodysplastic syndrome, which can develop into an acute myeloid leukemia.

15. The method according to claim 10, wherein the disorder, disease or medical condition is acute myeloid leukemia.

16. The method according to claim 10, wherein the at least one compound is:
(S)-N-(2-chloro-6-fluorophenyl)-4-(3,3-diethylureido)-5-fluoro-2-((1,1,1-trifluoropropan-2-yl)oxy)benzamide;
(S)-N-(4-((2-chloro-6-fluorophenyl)carbamoyl)-2-fluoro-5-((1,1,1-trifluoropropan-2-yl)oxy)phenyl)piperidine-1-carboxamide;
(S)-N-(2-chloro-6-fluorophenyl)-4-(3-ethyl-3-(2-hydroxyethyl)ureido)-5-fluoro-2-((1,1,1-trifluoropropan-2-yl)oxy)benzamide;
(S)-N-(2-chloro-6-fluorophenyl)-4-(3-ethyl-3-isopropylureido)-5-fluoro-2-((1,1,1-trifluoropropan-2-yl)oxy)benzamide;
(S)-N-(2-chloro-6-fluorophenyl)-4-(3-cyclopropyl-3-ethylureido)-5-fluoro-2-((1,1,1-trifluoropropan-2-yl)oxy)benzamide;
(S)-N-(2-chloro-6-fluorophenyl)-4-(3-ethyl-3-(2-methoxyethyl)ureido)-5-fluoro-2-((1,1,1-trifluoropropan-2-yl)oxy)benzamide;
(S)-N-(2-chloro-6-fluorophenyl)-4-(3-(2,2-difluoroethyl)-3-ethylureido)-5-fluoro-2-((1,1,1-trifluoropropan-2-yl)oxy)benzamide;
(S)-N-(2-chloro-6-fluorophenyl)-4-(3-ethylureido)-5-fluoro-2-((1,1,1-trifluoropropan-2-yl)oxy)benzamide;
(S)-N-(2-chloro-6-fluorophenyl)-5-fluoro-4-(3-isopropylureido)-2-((1,1,1-trifluoropropan-2-yl)oxy)benzamide;
(S)-N-(2-chloro-6-fluorophenyl)-4-(3,3-dimethylureido)-5-fluoro-2-((1,1,1-trifluoropropan-2-yl)oxy)benzamide;
(S)-N-(2-chloro-6-fluorophenyl)-4-(3-ethyl-3-methylureido)-5-fluoro-2-((1,1,1-trifluoropropan-2-yl)oxy)benzamide;
N-(4-((2-chloro-6-fluorophenyl)carbamoyl)-2-fluoro-5-(((S)-1,1,1-trifluoropropan-2-yl)oxy)phenyl)-2-(hydroxymethyl)azetidine-1-carboxamide;
N-(4-((2-chloro-6-fluorophenyl)carbamoyl)-2-fluoro-5-(((S)-1,1,1-trifluoropropan-2-yl)oxy)phenyl)-3-(hydroxymethyl)pyrrolidine-1-carboxamide;
(S)-N-(2-chloro-6-fluorophenyl)-5-fluoro-4-(3-methyl-3-propylureido)-2-((1,1,1-trifluoropropan-2-yl)oxy)benzamide;
(S)-N-(2-chloro-6-fluorophenyl)-4-(3-ethyl-3-propylureido)-5-fluoro-2-((1,1,1-trifluoropropan-2-yl)oxy)benzamide;
(S)-N-(2-chloro-6-fluorophenyl)-5-fluoro-4-(3-(2-fluoroethyl)-3-methylureido)-2-((1,1,1-trifluoropropan-2-yl)oxy)benzamide;
(S)-N-(2-chloro-6-fluorophenyl)-4-(3-(2-cyanoethyl)-3-methylureido)-5-fluoro-2-((1,1,1-trifluoropropan-2-yl)oxy)benzamide;
(S)-N-(2-chloro-6-fluorophenyl)-5-fluoro-4-(3-(2-hydroxyethyl)-3-methylureido)-2-((1,1,1-trifluoropropan-2-yl)oxy)benzamide;
(S)-N-(4-((2-chloro-6-fluorophenyl)carbamoyl)-2-fluoro-5-((1,1,1-trifluoropropan-2-yl)oxy)phenyl)pyrrolidine-1-carboxamide;
(S)-N-(4-((2-chloro-6-fluorophenyl)carbamoyl)-2-fluoro-5-((1,1,1-trifluoropropan-2-yl)oxy)phenyl)azetidine-1-carboxamide;
N-(4-((2-chloro-6-fluorophenyl)carbamoyl)-2-fluoro-5-(((S)-1,1,1-trifluoropropan-2-yl)oxy)phenyl)-2-(hydroxymethyl)pyrrolidine-1-carboxamide;
(S)-N-(4-((2-chloro-6-fluorophenyl)carbamoyl)-2-fluoro-5-((1,1,1-trifluoropropan-2-yl)oxy)phenyl)-3-fluoroazetidine-1-carboxamide;
(S)-N-(4-((2-chloro-6-fluorophenyl)carbamoyl)-2-fluoro-5-((1,1,1-trifluoropropan-2-yl)oxy)phenyl)-3-hydroxyazetidine-1-carboxamide;
(S)-N-(4-((2-chloro-6-fluorophenyl)carbamoyl)-2-fluoro-5-((1,1,1-trifluoropropan-2-yl)oxy)phenyl)-3-(hydroxymethyl)azetidine-1-carboxamide;
(S)-N-(4-((2-chloro-6-fluorophenyl)carbamoyl)-2-fluoro-5-((1,1,1-trifluoropropan-2-yl)oxy)phenyl)-3-(fluoromethyl)azetidine-1-carboxamide;
(S)-N-(2-chloro-6-fluorophenyl)-4-(3-(cyanomethyl)-3-methylureido)-5-fluoro-2-((1,1,1-trifluoropropan-2-yl)oxy)benzamide;
(R)-N-(4-((2-chloro-6-fluorophenyl)carbamoyl)-2-fluoro-5-(((S)-1,1,1-trifluoropropan-2-yl)oxy)phenyl)-2-(hydroxymethyl)pyrrolidine-1-carboxamide;
(S)-N-(4-((2-chloro-6-fluorophenyl)carbamoyl)-2-fluoro-5-(((S)-1,1,1-trifluoropropan-2-yl)oxy)phenyl)-2-(hydroxymethyl)pyrrolidine-1-carboxamide;
N-(4-((2-chloro-6-fluorophenyl)carbamoyl)-2-fluoro-5-(((S)-1,1,1-trifluoropropan-2-yl)oxy)phenyl)-3-methoxypyrrolidine-1-carboxamide;
N-(4-((2-chloro-6-fluorophenyl)carbamoyl)-2-fluoro-5-(((S)-1,1,1-trifluoropropan-2-yl)oxy)phenyl)-3-hydroxypyrrolidine-1-carboxamide;
N-(4-((2-chloro-6-fluorophenyl)carbamoyl)-2-fluoro-5-(((S)-1,1,1-trifluoropropan-2-yl)oxy)phenyl)-2-(methoxymethyl)pyrrolidine-1-carboxamide;
N-(4-((2-chloro-6-fluorophenyl)carbamoyl)-2-fluoro-5-(((S)-1,1,1-trifluoropropan-2-yl)oxy)phenyl)-3-fluoropyrrolidine-1-carboxamide;
(S)-N-(4-((2-chloro-6-fluorophenyl)carbamoyl)-2-fluoro-5-((1,1,1-trifluoropropan-2-yl)oxy)phenyl)-3,3-difluoropyrrolidine-1-carboxamide;
N-(4-((2-chloro-6-fluorophenyl)carbamoyl)-2-fluoro-5-(((S)-1,1,1-trifluoropropan-2-yl)oxy)phenyl)-2-(hydroxymethyl)-5-oxopyrrolidine-1-carboxamide;
(S)-N-(2-chloro-6-fluorophenyl)-4-(3,3-diethyl-1-methylureido)-5-fluoro-2-((1,1,1-trifluoropropan-2-yl)oxy)benzamide;
(R)-N-(4-((2-Chloro-4-methylpyridin-3-yl)carbamoyl)-2-fluoro-5-(((S)-1,1,1-trifluoropropan-2-yl)oxy)phenyl)-2-(hydroxymethyl)pyrrolidine-1-carboxamide;
(R)-N-(4-((5-Chloro-3-methyl-1H-pyrazol-4-yl)carbamoyl)-2-fluoro-5-(((S)-1,1,1-trifluoropropan-2-yl)oxy)phenyl)-2-(hydroxymethyl)pyrrolidine-1-carboxamide;
(R)-N-(2-Fluoro-4-((2-methoxy-4-methylpyridin-3-yl)carbamoyl)-5-(((S)-1,1,1-trifluoropropan-2-yl)oxy)phenyl)-2-(hydroxymethyl)pyrrolidine-1-carboxamide;
(R)-N-(4-((3-Chloro-2-methoxy-5-methylpyridin-4-yl)carbamoyl)-2-fluoro-5-(((S)-1,1,1-trifluoropropan-2-yl)oxy)phenyl)-2-(hydroxymethyl)pyrrolidine-1-carboxamide;
(R)-N-(2-Fluoro-4-((2-methoxy-3,5-dimethylpyridin-4-yl)carbamoyl)-5-(((S)-1,1,1-trifluoropropan-2-yl)oxy)phenyl)-2-(hydroxymethyl)pyrrolidine-1-carboxamide;
(S)-N-(2-Chloro-4-methylpyridin-3-yl)-4-(3-(2,2-difluoroethyl)-3-ethylureido)-5-fluoro-2-((1,1,1-trifluoropropan-2-yl)oxy)benzamide;
(S)-N-(5-Chloro-3-methyl-1H-pyrazol-4-yl)-4-(3-(2,2-difluoroethyl)-3-ethylureido)-5-fluoro-2-((1,1,1-trifluoropropan-2-yl)oxy)benzamide;
(S)-4-(3-(2,2-difluoroethyl)-3-ethylureido)-5-fluoro-N-(2-methoxy-4-methylpyridin-3-yl)-2-((1,1,1-trifluoropropan-2-yl)oxy)benzamide;
(S)-N-(3-Chloro-2-methoxy-5-methylpyridin-4-yl)-4-(3-(2,2-difluoroethyl)-3-ethylureido)-5-fluoro-2-((1,1,1-trifluoropropan-2-yl)oxy)benzamide;
(S)-4-(3-(2,2-Difluoroethyl)-3-ethylureido)-5-fluoro-N-(2-methoxy-3,5-dimethylpyridin-4-yl)-2-((1,1,1-trifluoropropan-2-yl)oxy)benzamide;
or a pharmaceutically acceptable salt, solvate, stereoisomer, isotopic variant, or N-oxide thereof.
